SEQUENCE LISTING (60 sequences)

SEQ ID NO: 1 - DNA, 141 bp, synthetic construct
  misc_feature 1..141: note = Synthetic
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3-4 - skipped (2 entries)

SEQ ID NO: 5 - DNA, 143 bp, synthetic construct
  misc_feature 1..143: note = Synthetic
  source 1..143: mol_type = other DNA
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 6 - DNA, 144 bp, synthetic construct
  misc_feature 1..144: note = Synthetic
  source 1..144: mol_type = other DNA
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 7 - DNA, 127 bp, synthetic construct
  misc_feature 1..127: note = Synthetic
  source 1..127: mol_type = other DNA
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 8 - DNA, 166 bp, synthetic construct
  misc_feature 1..166: note = Synthetic
  source 1..166: mol_type = other DNA
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 9 - DNA, 144 bp, synthetic construct
  misc_feature 1..144: note = Synthetic
  source 1..144: mol_type = other DNA
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 10 - DNA, 143 bp, synthetic construct
  misc_feature 1..143: note = Synthetic
  source 1..143: mol_type = other DNA
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 11 - DNA, 144 bp, synthetic construct
  misc_feature 1..144: note = Synthetic
  source 1..144: mol_type = other DNA
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 12 - DNA, 127 bp, synthetic construct
  misc_feature 1..127: note = Synthetic
  source 1..127: mol_type = other DNA
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 13 - DNA, 166 bp, synthetic construct
  misc_feature 1..166: note = Synthetic
  source 1..166: mol_type = other DNA
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 14 - DNA, 144 bp, synthetic construct
  misc_feature 1..144: note = Synthetic
  source 1..144: mol_type = other DNA
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 15 - DNA, 120 bp, synthetic construct
  misc_feature 1..120: note = Synthetic
  source 1..120: mol_type = other DNA
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 16 - DNA, 122 bp, synthetic construct
  misc_feature 1..122: note = Synthetic
  source 1..122: mol_type = other DNA
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 17 - DNA, 129 bp, synthetic construct
  misc_feature 1..129: note = Synthetic
  source 1..129: mol_type = other DNA
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 18 - DNA, 101 bp, synthetic construct
  misc_feature 1..101: note = Synthetic
  source 1..101: mol_type = other DNA
aggaacccctagtgatggagttggccactccctctctgcgcgctcgctcgctcactgaggctttgcctcagtgagcgagcgagcgcgcagctgcctgcagg

SEQ ID NO: 19 - DNA, 139 bp, synthetic construct
  misc_feature 1..139: note = Synthetic
  source 1..139: mol_type = other DNA
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 20 - DNA, 137 bp, synthetic construct
  misc_feature 1..137: note = Synthetic
  source 1..137: mol_type = other DNA
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 21 - DNA, 135 bp, synthetic construct
  misc_feature 1..135: note = Synthetic
  source 1..135: mol_type = other DNA
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 22 - DNA, 133 bp, synthetic construct
  misc_feature 1..133: note = Synthetic
  source 1..133: mol_type = other DNA
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 23 - DNA, 139 bp, synthetic construct
  misc_feature 1..139: note = Synthetic
  source 1..139: mol_type = other DNA
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 24 - DNA, 137 bp, synthetic construct
  misc_feature 1..137: note = Synthetic
  source 1..137: mol_type = other DNA
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 25 - DNA, 135 bp, synthetic construct
  misc_feature 1..135: note = Synthetic
  source 1..135: mol_type = other DNA
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 26 - DNA, 133 bp, synthetic construct
  misc_feature 1..133: note = Synthetic
  source 1..133: mol_type = other DNA
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 27 - DNA, 131 bp, synthetic construct
  misc_feature 1..131: note = Synthetic
  source 1..131: mol_type = other DNA
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 28 - DNA, 129 bp, synthetic construct
  misc_feature 1..129: note = Synthetic
  source 1..129: mol_type = other DNA
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 29 - DNA, 127 bp, synthetic construct
  misc_feature 1..127: note = Synthetic
  source 1..127: mol_type = other DNA
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 30 - DNA, 122 bp, synthetic construct
  misc_feature 1..122: note = Synthetic
  source 1..122: mol_type = other DNA
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 31 - DNA, 130 bp, synthetic construct
  misc_feature 1..130: note = Synthetic
  source 1..130: mol_type = other DNA
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 32 - DNA, 120 bp, synthetic construct
  misc_feature 1..120: note = Synthetic
  source 1..120: mol_type = other DNA
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 33 - DNA, 122 bp, synthetic construct
  misc_feature 1..122: note = Synthetic
  source 1..122: mol_type = other DNA
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 34 - DNA, 122 bp, synthetic construct
  misc_feature 1..122: note = Synthetic
  source 1..122: mol_type = other DNA
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 35 - DNA, 129 bp, synthetic construct
  misc_feature 1..129: note = Synthetic
  source 1..129: mol_type = other DNA
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 36 - DNA, 101 bp, synthetic construct
  misc_feature 1..101: note = Synthetic
  source 1..101: mol_type = other DNA
cctgcaggcagctgcgcgctcgctcgctcactgaggcaaagcctcagtgagcgagcgagcgcgcagagagggagtggccaactccatcactaggggttcct

SEQ ID NO: 37 - DNA, 139 bp, synthetic construct
  misc_feature 1..139: note = Synthetic
  source 1..139: mol_type = other DNA
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 38 - DNA, 137 bp, synthetic construct
  misc_feature 1..137: note = Synthetic
  source 1..137: mol_type = other DNA
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 39 - DNA, 135 bp, synthetic construct
  misc_feature 1..135: note = Synthetic
  source 1..135: mol_type = other DNA
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 40 - DNA, 133 bp, synthetic construct
  misc_feature 1..133: note = Synthetic
  source 1..133: mol_type = other DNA
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 41 - DNA, 139 bp, synthetic construct
  misc_feature 1..139: note = Synthetic
  source 1..139: mol_type = other DNA
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 42 - DNA, 137 bp, synthetic construct
  misc_feature 1..137: note = Synthetic
  source 1..137: mol_type = other DNA
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 43 - DNA, 135 bp, synthetic construct
  misc_feature 1..135: note = Synthetic
  source 1..135: mol_type = other DNA
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 44 - DNA, 133 bp, synthetic construct
  misc_feature 1..133: note = Synthetic
  source 1..133: mol_type = other DNA
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 45 - DNA, 131 bp, synthetic construct
  misc_feature 1..131: note = Synthetic
  source 1..131: mol_type = other DNA
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 46 - DNA, 129 bp, synthetic construct
  misc_feature 1..129: note = Synthetic
  source 1..129: mol_type = other DNA
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 47 - DNA, 127 bp, synthetic construct
  misc_feature 1..127: note = Synthetic
  source 1..127: mol_type = other DNA
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 48 - DNA, 122 bp, synthetic construct; identical to SEQ ID NO: 30
  misc_feature 1..122: note = Synthetic
  source 1..122: mol_type = other DNA

SEQ ID NO: 49 - protein, 1273 aa, SARS-CoV-2
  REGION 1..1273: note = misc_feature - severe acute respiratory syndrome coronavirus 2 isolate Wuhan-Hu-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 50 - DNA, 16 bp, synthetic construct
  misc_feature 1..16: note = Synthetic
  source 1..16: mol_type = other DNA
gagcgagcgagcgcgc

SEQ ID NO: 51 - DNA, 12 bp, synthetic construct
  misc_feature 1..12: note = Synthetic
  source 1..12: mol_type = other DNA
atcgaacgatcg

SEQ ID NO: 52 - DNA, 12 bp, synthetic construct
  misc_feature 1..12: note = Synthetic
  source 1..12: mol_type = other DNA
cgatcgttcgat

SEQ ID NO: 53 - DNA, 12 bp, synthetic construct
  misc_feature 1..12: note = Synthetic
  source 1..12: mol_type = other DNA
atcgaaccatcg

SEQ ID NO: 54-59 - skipped (6 entries)

SEQ ID NO: 60 - DNA, 16 bp, synthetic construct
  misc_feature 1..16: note = Synthetic
  source 1..16: mol_type = other DNA
gcgcgctcgctcgctc

(19) United States
(12) Patent Application Publication
(10) Pub. No.: US 2026/0284177 A1
(43) Pub. Date: Sep. 24, 2026

(54) HETEROLOGOUS PRIME BOOST VACCINE COMPOSITIONS AND METHODS OF USE

(71) Applicant: Generation Bio Co., Cambridge, MA (US)

(72) Inventors: Phillip Samayoa, Cambridge, MA (US); Constance Martin, Cambridge, MA (US); Matthew Manganiello, Cambridge, MA (US)

(21) Appl. No.: 18/846,711

(22) PCT Filed: Mar. 14, 2023
(86) PCT No.: PCT/US2023/015170

§ 371 Date: Sep. 13, 2024

Related U.S. Application Data
(60) Provisional application No. 63/319,505, filed on Mar. 14, 2022.

(51) Int. Cl. A61K 39/215; A61K 39/00; A61P 37/04

(52) U.S. Cl. CPC A61K 39/215; A61P 37/04; A61K 2039/53; A61K 2039/545; A61K 2039/575

(57) ABSTRACT

The application describes methods of inducing an immune response in a subject, comprising administering a prime-boost vaccine, wherein the priming vaccine comprises DNA (e.g., ceDNA) encoding a first peptide, and the boosting vaccine comprises (i) a ribonucleic acid (RNA), or (ii) a second peptide. Also provided are vaccine regimens, comprising a priming vaccine comprising DNA, wherein the DNA encodes a first peptide; and a boosting vaccine comprising (i) a ribonucleic acid (RNA), or (ii) a second peptide, wherein the RNA encodes the second peptide.

### RELATED APPLICATIONS

The instant application claims priority to U.S. Provisional Patent Application No. 63/319,505, filed on Mar. 14, 2022, the entire contents of which are expressly incorporated herein by reference.

### BACKGROUND

Generating a large population of antigen-specific memory CD8⁺ T cells to elicit long-lasting immune memory is a desirable goal for vaccine design against a variety of animal and human diseases. Typically, more than one immunization is required for a vaccine to induce efficient protection, and often an effective vaccine requires more than one time immunization in the form of prime-boost. For example, for pediatric population, up to five immunizations may be needed, as is the case for Diphtheria, Tetanus and Pertussis (DTP) vaccine, which is given three times during the first six months after birth, followed by a fourth dose in the second year of life, and a final boost between four and six years of age. Still, some of the vaccines need additional boosts even in adults who have already received the complete immunization series, for example, the Tetanus-diphtheria vaccine, for which a boost is recommended every 10 years throughout a person's lifespan. Such further administrations may be performed with the same vaccine (homologous boosting) or with a different vaccine (heterologous boosting). Homologous prime-boost immunizations that utilize re-administration of the same immunization agent have been used since the initial development of vaccines. Classic vaccination approaches relied on a homologous prime-boost regime and have traditionally been unable to elicit immune responses strong enough to tackle more challenging diseases. For example, although this method is usually effective in boosting the humoral response to antigen, it has been generally considered to be far less effective at generating increased numbers of CD8⁺ T cells due to rapid clearance of the homologous boosting agent by the primed immune system, and further fail to boost cellular immunity. One strategy to overcome this limitation has been the sequential administration of vaccines using different antigen delivery systems. This approach is called heterologous prime/boost.

While heterologous prime-boosts have been reported to increase responses in certain settings, not all combinations demonstrate improved immunity showing the importance of determining which combinations are effective. A number of factors, including selection of antigen, type of vector, delivery route, dose, adjuvant, boosting regimen, the order of vector injection, and the intervals between different vaccinations influence the outcome of prime-boost immunization approaches, making results hard to predict. Finding vaccine combinations that elicit broad, durable, and long-lasting immunity are important for conferring robust protection.

Recombinant AAV (rAAV) is perhaps the best studied vector for gene transfer in humans, with hundreds of clinical trials demonstrating safety of transduction. Adeno-associated viruses (AAVs) belong to the Parvoviridae family and more specifically constitute the Dependoparvovirus genus. Vectors derived from AAV (i.e., rAVV or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the replication (rep) gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see (ii)), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. Particularly related to antibody delivery, the packaging limitation of AAV represents a significant challenge for the efficient delivery of both heavy and light chains that form the natural antibody structure. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Preexisting immunity can severely limit the efficiency of transduction. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Adenovirus vectors whereby the vector expresses an unknown antigenic protein have been well studied for gene and cancer therapy and vaccines. Apart from its extensive safety profile, the advantages of utilizing an adenovirus vector are that it is relatively stable, easy to attain high titers and able to infect multiple cell lines which attributes to its potency. Even though recombinant adenoviral vectors are widely used today thanks to its high transduction efficiency and transgene expression, there is likelihood for pre-existing immunity against the vector, because most of the population has been exposed to adenovirus (Id). This has been proven detrimental in a human immunodeficiency virus (HIV-1) phase IIb vaccine trial in which the vector-based vaccines provided favorable conditions for HIV-1 replication (Smaill, F. et al., Sci. Transl. Med. (2013) 5: 205).

There remains a need in the art for heterologous prime boost regimens that provide robust immunogenicity without inducing anti-vector immunity.

### SUMMARY

The disclosure provides prime-boost compositions and methods comprising a priming vaccine comprising a first peptide encoded by a DNA and a booster vaccine that comprises a second peptide.

In some embodiments, the second peptide is encoded by an mRNA. According to some embodiments, the DNA may be in the form of, e.g., a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. According to some embodiments, the priming vaccine comprises plasmid DNA. According to some embodiments, the priming vaccine comprises closed-ended linear duplex DNA (ceDNA).

As demonstrated in the Examples herein, a ceDNA vaccine platform can be successfully employed as a priming vaccine in heterologous prime/boost regimens for eliciting and enhancing both humoral and cellular responses to an encoded model antigen. The results presented herein suggest that the heterologous prime-boost regimen can confer synergistically stronger responses to antigens and greater protection than immunization with the same vaccine alone. It is a finding of the present disclosure that by priming with a DNA priming platform, e.g., a ceDNA vector platform, and boosting with an mRNA based or a peptide based platform (a heterologous prime-boost regimen), immune responses can be improved. The heterologous prime-boosts using two immunologically different platforms as described herein were advantageously engineered using a DNA (e.g., ceDNA) as a priming vaccine such that the follow-on administrations (boost) activates the immune system in different ways that synergize with the initial administration (prime). Further, the prime boost compositions and methods described herein generate increased CD8⁺ memory T cell responses.

The application of the prime-boost compositions and methods described herein, wherein the priming vaccine comprises a DNA (e.g., a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vectors) wherein the DNA encodes a first peptide, and the boosting vaccine, comprising an RNA encoding a second peptide, or a second peptide, is useful to: treat, prevent or reduce the severity of a disease or disorder in a subject, be minimally invasive in delivery, be repeatable and dosed-to-effect, have rapid onset of therapeutic effect, and/or result in sustained expression of antigen, or immunogenic peptide.

Unlike traditional vaccines, which are manufactured ex vivo and may trigger unwanted cellular responses, the ceDNA vaccines used herein as prime vaccines are presented to the cellular system in a more native fashion. By employing a ceDNA vector to deliver a transgene (e.g., a nucleic acid sequence) encoding an antigen to cells or tissues, the adaptive immune response is bypassed, and the desired antibody specificities are produced without the use of immunization or passive transfer.

That is, the ceDNA vector enters the cell via endocytosis, then escapes from the endosomal compartment and is transported to the nucleus. The transcriptionally active ceDNA episome results in the expression of antigens that may then be secreted from the cell into the circulation. The ceDNA vector may therefore enable continuous, sustained and long-term delivery of antibodies (e.g., the therapeutic antibodies, or antigen-binding fragments therein, described herein) administered by a single injection. This is particularly advantageous in the context of the described nucleic acid vaccine compositions, where the DNA prime vaccines show a slower increase in expression and a more sustained expression as compared to mRNA vaccines which although may show more an increased initial expression, the expression was not sustained, and decreased more rapidly.

According to some aspects, the disclosure provides a method of inducing an immune response against a first peptide and a second peptide in a subject, comprising administering a priming vaccine comprising a deoxyribonucleic acid (DNA) to the subject, wherein the DNA encodes a first peptide; and administering a boosting vaccine comprising (i) a ribonucleic acid (RNA), wherein the RNA encodes the second peptide or (ii) a second peptide to the subject, thereby inducing the immune response against the first peptide and the second peptide in the subject. According to some embodiments, the priming vaccine comprises DNA encoding the first peptide and the boosting vaccine comprises RNA encoding the second peptide. According to some embodiments, the priming vaccine comprises DNA encoding the first peptide and the boosting vaccine comprises the second peptide. According to further embodiments of any of the embodiments herein, the DNA comprises a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. According to further embodiments of any of the embodiments herein, the first and the second peptide are derived from a bacterial, a viral, a fungal or a parasitic infectious agent. According to further embodiments of any of the embodiments herein, the first and the second peptide are derived from the same pathogenic organism. According to further embodiments of any of the embodiments herein, the first and the second peptide are the same in the priming vaccine and the boosting vaccine. According to further embodiments of any of the embodiments herein, at least one of the epitopes of the first and the second peptide are different in the priming and the boosting vaccine. According to some embodiments, the DNA comprises a capsid-free closed ended DNA (ceDNA) vector comprising at least one nucleic acid sequence between flanking inverted terminal repeats (ITRs), wherein the at least one nucleic acid sequence encodes the peptide. According to some embodiments, the first and/or the second peptide is a tumor associated antigen or is associated with an autoimmune condition. According to further embodiments of any of the aspects or embodiments herein, the first or the second peptide is selected from one or more of those set forth in Tables 1-8. According to further embodiments of any of the embodiments herein, the ceDNA vector further comprises a promoter sequence linked to the at least one nucleic acid sequence. According to further embodiments of any of the embodiments herein, the ceDNA vector comprises at least one poly A sequence. According to further embodiments of any of the embodiments herein, the ceDNA vector comprises a 5′ UTR and/or an intron sequence. According to further embodiments of any of the embodiments herein, the ceDNA vector comprises a 3′ UTR sequence. According to further embodiments of any of the embodiments herein, the ceDNA vector comprises an enhancer sequence. According to further embodiments of any of the embodiments herein, at least one of the ITRs comprises a functional terminal resolution site and a Rep binding site. According to further embodiments of any of the embodiments herein, at least one or both of the ITRs are from a virus selected from a Parvovirus, a Dependovirus, and an adeno-associated virus (AAV). According to further embodiments of any of the embodiments herein, the flanking ITRs are symmetric or asymmetric with respect to each other. According to some embodiments, the flanking ITRs are symmetric or substantially symmetric. According to some embodiments, the flanking ITRs are asymmetric. According to further embodiments of any of the embodiments herein, one of the flanking ITRs are wild-type, or both of the flanking ITRs are wild-type ITRs. According to further embodiments of any of the embodiments herein, the flanking ITRs are derived from different viral serotypes. According to further embodiments of any of the embodiments herein, the flanking ITRs are selected from any pair of viral serotypes shown in Table 8. According to further embodiments of any of the embodiments herein, the one or both of the ITRs comprises a sequence selected from one or more of the sequences in Table 9. According to further embodiments of any of the embodiments herein, the at least one of the flanking ITRs is altered from a wild-type AAV ITR sequence by a deletion, an addition, or a substitution that affects the overall three-dimensional conformation of the ITR. According to further embodiments of any of the embodiments herein, one or both of the flanking ITRs are derived from an AAV serotype selected from the group consisting of AAV1, AAV2, AAV3, AAV4, AAV5, AAV6, AAV7, AAV8, AAV9, AAV10, AAV11, and AAV12. According to further embodiments of any of the embodiments herein, the one or both of the flanking ITRs are synthetic. According to further embodiments of any of the embodiments herein, one of the flanking ITRs are not a wild-type ITR, or both of the flanking ITRs are not wild-type ITRs.

According to further embodiments of any of the embodiments herein, one or both of the flanking ITRs are modified by a deletion, an insertion, and/or a substitution in at least one of the ITR regions selected from A, A′, B, B′, C, C′, D, and D′. According to some embodiments, the deletion, insertion, and/or substitution results in the deletion of all or part of a stem-loop structure a formed by the A, A′, B, B′, C, or C′ regions. According to further embodiments of any of the embodiments herein, one or both of the flanking ITRs are modified by a deletion, n insertion, and/or a substitution that results in the deletion of all or part of a stem-loop structure formed by the B and B′ regions. According to further embodiments of any of the embodiments herein, one or both of the flanking ITRs are modified by a deletion, an insertion, and/or a substitution that results in the deletion of all or part of a stem-loop structure formed by the C and C′ regions. According to further embodiments of any of the embodiments herein, one or both of the flanking ITRs are modified by a deletion, an insertion, and/or substitution that results in the deletion of part of a stem-loop structure formed by the B and B′ regions and/or part of a stem-loop structure formed by the C and C′ regions. According to further embodiments of any of the embodiments herein, one or both of the flanking ITRs comprise a single stem-loop structure in the region that, in a wild-type ITR, would comprise a first stem-loop structure formed by the B and B′ regions and a second stem-loop structure formed by the C and C′ regions. According to further embodiments of any of the embodiments herein, one or both of the flanking ITRs comprise a single stem and two loops in the region that, in a wild-type ITR, would comprise a first stem-loop structure formed by the B and B′ regions and a second stem-loop structure formed by the C and C′ regions. According to further embodiments of any of the embodiments herein, one or both of the flanking ITRs comprise a single stem and a single loop in the region that, in a wild-type ITR, would comprise a first stem-loop structure formed by the B and B′ regions and a second stem-loop structure formed by the C and C′ regions. According to further embodiments of any of the embodiments herein, both of the flanking ITRs are altered in a manner that results in an overall three-dimensional symmetry when the ITRs are inverted relative to each other. According to further embodiments of any of the embodiments herein, the DNA is delivered in a lipid nanoparticle (LNP). According to further embodiments of any of the embodiments herein, the RNA is delivered in a lipid nanoparticle (LNP). According to further embodiments of any of the embodiments herein, the RNA is a messenger RNA (mRNA). According to further embodiments, the he RNA comprises at least one nucleotide analogue. According to further embodiments of any of the embodiments herein, the immune response is an antibody response. According to further embodiments of any of the embodiments herein, the immune response is a T cell response. According to other further embodiments, the immune response is a memory (CD8⁺) T cell response. According to some embodiments of the aspects and embodiments described herein, the method comprises administering the boosting vaccine at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 14 weeks, at least about 16 weeks, at least about 1-2 weeks, at least about 2-3 weeks, at least about 3-4 weeks, at least about 4-5 weeks, at least about 5-6 weeks, at least about 6-7 weeks, at least about 7-8 weeks, at least about 8-9 weeks, at least about 9-10 weeks, at least about 10-11 weeks, at least about 11-12 weeks, at least about 12-13 weeks, at least 13-14 weeks, at least about 14-15 weeks, or at least about 15-16 weeks after administering the priming vaccine. According to other further embodiments of any of the embodiments herein, the method comprises administering the boosting vaccine at least 8 weeks after administering the priming vaccine. According to other embodiments of any of the embodiments herein, the method comprises administering the boosting vaccine about 8 weeks after administering the priming vaccine. According to some embodiments of the aspects and embodiments described herein, the interval between the administering of the priming vaccine and the administering of the boosting vaccine is at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, or at least about 112 days. According to further embodiments, the interval between the administration of the priming vaccine and the administration of the boosting vaccine is about 64 days. According to some embodiments of the aspects and embodiments described herein, the method comprises administering two or more doses of the boosting vaccine to the subject. According to some embodiments of the aspects and embodiments described herein, he method comprises administering each dose of boosting vaccine at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 14 weeks, at least about 16 weeks, at least about 1-2 weeks, at least about 2-3 weeks, at least about 3-4 weeks, at least about 4-5 weeks, at least about 5-6 weeks, at least about 6-7 weeks, at least about 7-8 weeks, at least about 8-9 weeks, at least about 9-10 weeks, at least about 10-11 weeks, at least about 11-12 weeks, at least about 12-13 weeks, at least 13-14 weeks, at least about 14-15 weeks, or at least about 15-16 weeks after administering the previous vaccine. According to some embodiments of the aspects and embodiments described herein, the interval between the administering of the each dose of boosting vaccine and the administering of the previous vaccine is at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, or at least about 113 days. According to further embodiments of any of the embodiments herein, the subject has a bacterial infection, a viral infection, a parasitic infection or a fungal infection. According to further embodiments of any of the embodiments herein, the subject has cancer. According to further embodiments of any of the embodiments herein, the subject has an autoimmune disease or disorder. According to further embodiments of any of the embodiments herein, one or more of the priming vaccine or the boosting vaccine comprises a pharmaceutically acceptable carrier. According to some embodiments, at least one of the priming vaccine and the boosting vaccine compositions further comprises an adjuvant. According to further embodiments of any of the embodiments herein, at least one of the priming vaccine and the boosting vaccine is administered by a route selected from intramuscular, intraperitoneal, buccal, inhalation, intranasal, intrathecal, intravenous, subcutaneous, intradermal, and intratumoral, or is administered to the interstitial space of a tissue.

According to some aspects, the disclosure provides a vaccine regimen comprising a priming vaccine comprising a deoxyribonucleic acid (DNA) DNA, wherein the DNA encodes a first peptide followed by a boosting vaccine comprising (i) a ribonucleic acid (RNA) that encodes a second peptide, or (ii) a second peptide. According to some embodiments, the priming vaccine comprises an amount of DNA encoding an immunologically effective amount of the first peptide, and the boosting vaccine comprises an immunologically effective amount of RNA encoding the second peptide. According to some embodiments, the priming vaccine comprises an amount of DNA encoding an immunologically effective amount of the first a peptide and the boosting vaccine comprises an immunologically effective amount of the second peptide. According to further embodiments of any of the embodiments herein, the DNA comprises a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. According to further embodiments of any of the embodiments herein, the first peptide and the second peptide are derived from a bacterial infectious agent, a viral infectious agent, a fungal infectious agent or a parasitic infectious agent. According to further embodiments of any of the embodiments herein, the first peptide and the second peptide are derived from the same pathogenic organism. According to further embodiments of any of the embodiments herein, the first peptide and the second peptide are the same in the priming vaccine and the boosting vaccine. According to further embodiments of any of the embodiments herein, at least one of the epitopes of the first peptide and the second peptide are different in the priming and the boosting vaccine. According to some embodiments, the DNA comprises a capsid-free closed ended DNA (ceDNA) vector comprising at least one nucleic acid sequence between flanking inverted terminal (ITRs), wherein the at least one nucleic acid sequence encodes the first peptide. According to some embodiments, the first peptide and/or the second peptide is a tumor associated antigen. According to some embodiments, the first peptide and/or the second peptide is associated with an autoimmune condition. According to further embodiments of any of the embodiments herein, the first peptide or the second peptide is selected from one or more of those set forth in Tables 1-8.

The disclosure also features a method of treating a subject with a bacterial infection, a viral infection a parasitic infection or a fungal infection, comprising performing the method of any of the aspects or embodiments herein or administering to the subject the vaccine regimen of any one of the aspects and embodiments herein.

The disclosure also features a method of treating a subject with a cancer, comprising performing the method of any of the aspects or embodiments herein or administering to the subject the vaccine regimen of any one of the aspects and embodiments herein

The disclosure also features a method of treating a subject with an autoimmune disease or disorder, comprising performing the method of any of the aspects or embodiments herein or administering to the subject the vaccine regimen of any one of the aspects and embodiments herein.

The disclosure also features a method of preventing a bacterial infection, a viral infection, a parasitic infection or a fungal infection in a subject, comprising performing the method of any of the aspects or embodiments herein or administering to the subject the vaccine regimen of any one of the aspects and embodiments herein.

The disclosure also features a method of preventing cancer in a subject, comprising performing the method of any of the aspects or embodiments herein or administering to the subject the vaccine regimen of any one of the aspects and embodiments herein.

The disclosure also features a method of preventing an autoimmune disease in a subject, comprising performing the method of any of the aspects or embodiments herein or administering to the subject the vaccine regimen of any one of the aspects and embodiments herein.

According to further embodiments of any of the embodiments herein, the method comprises administering two or more doses of the boosting vaccine to the subject. According to further embodiments of any of the embodiments herein, the method comprises administering the boosting vaccine about 8 weeks after administering the priming vaccine. According to further embodiments of any of the embodiments herein, the method further comprises administering to the subject one or more additional therapeutic agents.

According to other aspects, the priming vaccine and the boosting vaccine are each formulated in a pharmaceutical composition. According to some embodiments, one or both of the priming vaccine and the boosting vaccine further comprise one or more additional therapeutic agents. According to other further embodiments, one or both of the priming vaccine and the boosting vaccine further comprise a lipid. According to some embodiments, the lipid is a lipid nanoparticle (LNP). According to further embodiments, one or both of the priming vaccine and the boosting vaccine are lyophilized.

The disclosure also features a pharmaceutical composition comprising the vaccine regimen of any one of the aspects and embodiments herein. According to some embodiments, the pharmaceutical composition further comprises one or more additional therapeutic agents.

The disclosure also features a composition comprising the vaccine regimen of any one of the aspects and embodiments herein, and a lipid. According to some embodiments, the lipid is a lipid nanoparticle (LNP). According to further embodiments of any of the embodiments herein, the composition is lyophilized.

In other aspects, the disclosure provides a kit comprising the vaccine regimen of any one of the aspects and embodiments herein, and instructions for use. In other aspects, the disclosure provides a kit comprising one or both of the priming vaccine and the boosting vaccine of any one of the aspects and embodiments herein, and instructions for use. In some embodiments, the kit comprises a lipid.

These and other aspects of the disclosure are described in further detail below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1 is a graph that depicts spike protein antibody titer as determined on Day 49 of the study described in Example 5.

FIG. 2 is a graph that depicts spike protein antibody titer as determined on day 77 of the study described in Example 5.

FIG. 3 is a graph that depicts spike protein antibody titer as determined on day 105 of the study described in Example 5.

FIG. 4 is a graph that depicts the percentage of CD8⁺ T cells in the population that were IFNγ⁺, IFNγ⁺ and CD107⁺, IFNγ⁺ and TNFα⁺ or IL4⁺ at assay day 77.

FIG. 5 is a graph that depicts spike protein antibody titer as determined at day 21 and day 49 of the study described in Example 6.

FIG. 6 is a graph that depicts the percentage of IFNγ⁺ antigen-specific memory CD8⁺ T cells in mouse spleen cell suspensions 8 weeks after immunization with mRNA, ceDNA, or plasmids encoding the COVID spike protein.

FIG. 7 is a graph that depicts the percentage of IFNγ⁺ antigen-specific memory CD8⁺ T cells in mice primed and boosted at either 4, 6, or 8 week intervals with ceDNA-ceDNA, mRNA-mRNA, or ceDNA-mRNA regimens.

FIG. 8 is a graph that depicts the percentage of IFNγ⁺ antigen-specific memory CD8⁺ T cells after heterologous prime-boost regimens of 0.3 μg mRNA-3 μg mRNA, 1 μg mRNA-3 μg mRNA, 3 μg mRNA-3 μg mRNA, 3 μg ceDNA-3 μg mRNA, and 10 μg ceDNA-3 μg mRNA.

### DETAILED DESCRIPTION

The present disclosure generally relates to the use of compositions and methods for inducing an immune response in a subject using heterologous prime-boost immunization regimens. Included herein are methods of inducing an immune response against a first peptide and a second peptide in a subject, comprising administering a priming vaccine comprising a deoxyribonucleic acid (DNA) DNA to the subject, wherein the DNA encodes a first peptide; and administering a boosting vaccine comprising (i) a ribonucleic acid (RNA), or (ii) a second peptide to the subject, wherein the RNA encodes the second peptide, thereby inducing the immune response against the first peptide and the second peptide in the subject, can be used prophylactically and/or therapeutically. In some embodiments, the compositions and methods disclosed herein can be used for the production of a molecule of interest, e.g., a therapeutic polypeptide, in a subject.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-O-911910-19-3); Robert S. Porter et al. (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “immunization” or “active immunization” as used herein refers to the production of active immunity, meaning immunity resulting from a naturally acquired infection or intentional vaccination (artificial active immunity).

The term “adjuvant” as used herein, is meant to refer to an agent that, when used in combination with a specific immunogen in a formulation, will augment or otherwise alter or modify the resultant immune response. Modification of the immune response includes intensification or broadening the specificity of the immune response (e.g., either or both the antibody and cellular immune responses). Modification of the immune response can also mean decreasing or suppressing certain antigen-specific immune responses.

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B cell epitope will include at least about 5 amino acids but can be as small as 3-4 amino acids. A T cell epitope, such as a CTL epitope, will include at least about 7-9 amino acids, and a helper T cell epitope at least about 12-20 amino acids. Normally, an epitope will include between about 7 and 15 amino acids, inclusive, such as, 9, 10, 11, 12, 13, 14 or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “epitope” may be also referred to as an antigenic determinant, is a molecular determinant (e.g., polypeptide determinant) that can be specifically bound by a binding agent, immunoglobulin or T cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. Epitopes may be defined as structural or functional. Functional epitopes are generally a subset of the structural epitopes and have those residues that directly contribute to the affinity of the interaction. Epitopes may be linear or conformational, that is, composed of non-linear amino acids. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody specifically binds an antigen when it recognizes its target antigen in a complex mixture of proteins and/or macromolecules. For example, antibodies are said to “bind to the same epitope” if the antibodies cross-compete (one prevents the binding or modulating effect of the other).

As used herein, the term “autoimmune disorders” refers generally to conditions in which a subject's immune system attacks the body's own cells, causing tissue destruction. Autoimmune disorders may be diagnosed using blood tests, cerebrospinal fluid analysis, electromyogram (measures muscle function), and magnetic resonance imaging of the brain, but antibody testing in the blood, for self-antibodies (or auto-antibodies) is particularly useful. Usually, IgG class antibodies are associated with autoimmune diseases.

The terms “B lymphocyte” or “B cell” are used interchangeably to refer to a broad class of lymphocytes, which are precursors of antibody-secreting cells, that express clonally diverse cell surface immunoglobulin (Ig) receptors (BCRs) recognizing specific antigenic epitopes. Mammalian B cell development encompasses a continuum of stages that begin in primary lymphoid tissue (e.g., human fetal liver and fetal/adult marrow), with subsequent functional maturation in secondary lymphoid tissue (e.g., human lymph nodes and spleen). The functional/protective end point is antibody production by terminally differentiated plasma cells. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). (LeBien, TW & TF Tedder, B lymphocytes: how they develop and function. Blood (2008) 112 (5): 1570-80).

As used herein, the term “cancer” refers to diseases in which abnormal cells divide without control and are able to invade other tissues. There are more than 100 different types of cancer. Most cancers are named for the organ or type of cell in which they start—for example, cancer that begins in the colon is called colon cancer; cancer that begins in melanocytes of the skin is called melanoma. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system (CNS) cancers (meaning cancers that begin in the tissues of the brain and spinal cord). The term “myelodysplastic syndrome” refers to a type of cancer in which the bone marrow does not make enough healthy blood cells (white blood cells, red blood cells, and platelets) and there are abnormal cells in the blood and/or bone marrow. Myelodysplastic syndrome may become acute myeloid leukemia (AML). In certain embodiments, the cancer is selected from cancers including, but not limited to, ACUTE lymphoblastic leukemia (ALL), ACUTE myeloid leukemia (AML), anal cancer, bile duct cancer, bladder cancer, bone cancer, bowel cancer, brain tumor, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumor (GTT), hairy cell leukemia, head and neck cancer, Hodgkin lymphoma, kidney cancer, laryngeal cancer, leukemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non hodgkin lymphoma (NHL), esophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

As used herein, the term “cross-protection” is used to describe immunity against at least two subgroups, subtypes, strains and/or variants of a virus, bacteria, parasite or other pathogen with a single inoculation with one subgroup, subtype, strain and/or variant thereof.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “effector cell” as used herein refers to a cell that carries out a final response or function. The main effector cells of the immune system, for example, are activated lymphocytes and phagocytes.

The term “herd immunity” as used herein refers to protection conferred to unvaccinated individuals in a population produced by vaccination of others and reduction in the natural reservoir for infection.

The term “heterosubtypic immunity” (“HSI”) as used herein refers to immunity based on immune recognition of antigens conserved across all viral strains.

The term “heterotypic” as used herein is used to refer to being of a different or unusual type or form (e.g., different subgroup, subtype, strain and/or variant of a virus, bacteria, parasite or other pathogen).

The term “homotypic” as used herein is used to refer to being of the same type or form, e.g., same subgroup, subtype, strain and/or variant of a virus, bacteria, parasite or other pathogen.

The terms “immune response” and “immune-mediated” as used herein, are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T lymphocytes and/or other white blood cells. One important aspect of cellular immunity involves an antigen-specific response by cytolytic T cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T cells. Helper T cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T cells and/or other white blood cells, including those derived from CD4⁺ and CD8⁺ T cells. Hence, an immunological response may include one or more of the following effects: the production of antibodies by B cells; and/or the activation of suppressor T cells and/or γδ T cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immune phenotype” or “immunotype” as used herein refers to the collective frequency of various immune cell populations and their functional responses to stimuli (cell signaling and antibody responses). (See Kaczorowski, K J et al. Proc. Nat. Acad. Sci. USA (2017)).

The term “immune system” as used herein refers to the body's system of defenses against disease, which comprises the innate immune system and the adaptive immune system. The innate immune system provides a non-specific first line of defense against pathogens. It comprises physical barriers (e.g., the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunodominant epitope” as used herein refers to the epitope against which the majority of antibodies is raised, or to which the majority of T cells responds.

The term “immunogenic amount” or “immunologically effective amount” as used herein refers to the amount of an active component (such as an immunogenic peptide) sufficient to elicit either an antibody or a T cell response, or both, sufficient to have a beneficial effect, e.g., a prophylactic or therapeutic effect, on the subject.

The term “immunological repertoire” refers to the collection of transmembrane antigen-receptor proteins located on the surface of T and B cells. (Benichou, J. et al. Immunology (2011) 135:183-191)) The combinatorial mechanism that is responsible for encoding the receptors does so by reshuffling the genetic code, with a potential to generate more than 1018 different T cell receptors (TCRs) in humans (Venturi, Y. et al. Nat. Rev. Immunol. (2008) 8: 231-8) and a much more diverse B cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen.

A peptide, oligopeptide, polypeptide, protein, or polynucleotide coding for such a molecule is “immunogenic” and thus an immunogen within the present disclosure if it is capable of inducing an immune response. In the present disclosure, immunogenicity is more specifically defined as the ability to induce a CTL-mediated response. Thus, an immunogen would be a molecule that is capable of inducing an immune response, and in the present disclosure, a molecule capable of inducing a CTL response. An immunogen may have one or more isoforms, sequence variants, or splice variants that have equivalent biological and immunological activity, and are thus also considered for the purposes of this disclosure to be immunogenic equivalents of the original, natural polypeptide.

The term “priming” or “prime” is meant to refer to the administration of a vaccine (a “priming vaccine”) or an immunogenic composition which induces a higher level of an immune response, when followed by a subsequent administration of the same or of a different vaccine immunogenic composition, than the immune response obtained by administration with a single vaccine or immunogenic composition. According to some embodiments, a “priming vaccine” is a DNA priming vaccine. According to some embodiments, a DNA priming vaccine may be in the form of, e.g., a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. According to some embodiments, the priming vaccine comprises closed-ended linear duplex DNA (ceDNA). According to some embodiments, the priming vaccine comprises plasmid DNA.

The term “boosting” or “boost” is meant to refer to the administration of a subsequent vaccine (a “boosting vaccine”) or immunogenic composition after the administration of a priming vaccine or immunogenic composition, wherein the subsequent administration produces a higher level of immune response than an immune response to a single administration of a vaccine or an immunogenic composition. A boosting vaccine can be the same or of a different vaccine immunogenic composition of the priming vaccine or immunogenic composition.

The term “heterologous prime boost” as used herein is meant to refer to a regimen comprising priming the immune response with an immunogenic peptide or an antigen and subsequent boosting of the immune response with an immunogenic peptide or an antigen delivered by a different molecule and/or vector. For example, heterologous prime boost regimens of the invention include priming with a ceDNA vector and boosting with an mRNA vector as well as priming with a ceDNA vector and boosting with an immunogenic peptide. Heterologous prime boost regimens of the invention can also include, for example, priming with a plasmid DNA and boosting with an mRNA vector as well as priming with a plasmid DNA and boosting with an immunogenic peptide

The term “specifically binds,” as used herein refers to the ability of a polypeptide or polypeptide complex to recognize and bind to a ligand in vitro or in vivo while not substantially recognizing or binding to other molecules in the surrounding milieu. In some embodiments, specific binding can be characterized by an equilibrium dissociation constant of at least about 1×10⁶M or less (e.g., a smaller equilibrium dissociation constant denotes tighter binding). Methods for determining whether two molecules specifically bind are well known in the art and include, for example, equilibrium dialysis, surface plasmon resonance, and the like.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jonsson et al. (1993) Ann. Biol. Clin. 51:19; Jonsson et al. (1991) Biotechniques 11:620-627; Johnsson et al. (1995) J. Mol. Recognit. 8:125; and Johnnson et al. (1991) Anal. Biochem. 198:268.

As used herein, the terms “heterologous nucleic acid sequence” and “transgene” are used interchangeably and refer to a nucleic acid of interest (other than a nucleic acid encoding a capsid polypeptide) that is incorporated into and may be delivered and expressed by a ceDNA vector as disclosed herein. According to some embodiments, the term “heterologous nucleic acid” is meant to refer to a nucleic acid (or transgene) that is not present in, expressed by, or derived from the cell or subject to which it is contacted.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The terms DNA and DNA molecule(s) are used interchangeably herein and are meant to refer to DNA that may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. According to preferred embodiments, DNA of the priming vaccine comprises a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular antigen or immunogenic peptide, is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses Parvoviruses and Dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleic acid sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. According to some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change According to some or more of A, C, C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. According to some embodiments, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). According to some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 91%, 92%, 93%, 94%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. According to some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification According to some ITR reflected in the corresponding position in the cognate ITR from a different serotype. According to some embodiments, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 90%, 91%, 92%, 93%, 94%, 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, an “Internal ribosomal entry site” (IRES) is meant to refer to a nucleotide sequence (>500 nucleotides) that allows for initiation of translation in the middle of an mRNA sequence (Kim, J I T. et al., 2011. PLoS One 6(4): 8556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

As used herein, “2A peptides” are meant to refer to small self-cleaving peptides derived from viruses such as foot-and-mouth disease virus (F2A), porcine teschovirus-1 (P2A), osea asigna virus (T2A), or equine rhinitis A virus (E2A). The 2A designation refers specifically to a region of picomavirus poiyproteins that lead to a ribosomal skip at the glycyl-prolyl bond in the O terminus of the 2A peptide (Kim, J. I T. et al. 2011. PLoS One 6(4); the contents of which are herein incorporated by reference in its entirety). This skip results in a cleavage between the 2A peptide and its immediate downstream peptide.

The term “flanking” refers to a relative position of one nucleic acid sequence with respect to another nucleic acid sequence. Generally, in the sequence ABC, B is flanked by A and C. The same is true for the arrangement AxBxC. Thus, a flanking sequence precedes or follows a flanked sequence but need not be contiguous with, or immediately adjacent to the flanked sequence. According to some embodiments, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. According to some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. According to some embodiments, ceDNA spacer regions keep two functional elements at a desired distance for optimal functionality. According to some embodiments, ceDNA spacer regions provide or add to the genetic stability of the ceDNA genome within e.g., a plasmid or baculovirus. According to some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis—acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′, an RBS sequence identified in AAV2. Any known RBS sequence may be used in the embodiments of the disclosure, including other known AAV RBS sequences and other naturally known or synthetic RBS sequences. Without being bound by theory it is thought that he nuclease domain of a Rep protein binds to the duplex nucleic acid sequence GCTC, and thus the two known AAV Rep proteins bind directly to and stably assemble on the duplex oligonucleotide, 5′-(GCGC)(GCTC)(GCTC)(GCTC)-3′. In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. According to some embodiments, a TRS minimally encompasses a non-base-paired thymidine. According to some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the disclosure, including other known AAV TRS sequences and other naturally known or synthetic TRS sequences such as AGTT, GGTTGG, AGTTGG, AGTTGA, and other motifs such as RRTTRR.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference. As used herein, the terms “ceDNA vector” and “ceDNA” are used interchangeably and refer to a closed-ended DNA vector comprising at least one terminal palindrome. According to some embodiments, the ceDNA comprises two covalently-closed ends.

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. According to some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a transgene (e.g., a nucleic acid encoding an antibody or antigen-binding fragment thereof as described herein). Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. According to some embodiments, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. According to some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. According to some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background. According to some embodiments, a promoter of the disclosure is a liver specific promoter.

The term “enhancer” as used herein refers to a cis-acting regulatory sequence (e.g., 10-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, according to some embodiments, an enhancer can be one naturally associated with a nucleic acid sequence, located either downstream or upstream of that sequence.

According to some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. According to some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. According to some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csnl polypeptide) and/or regulate translation of an encoded polypeptide.

The term “open reading frame (ORF)” as used herein is meant to refer to a sequence of several nucleotide triplets which may be translated into a peptide or protein. An open reading frame preferably contains a start codon, i.e. a combination of three subsequent nucleotides coding usually for the amino acid methionine (ATG), at its 5′-end and a subsequent region which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present disclosure is preferably a nucleotide sequence, consisting of a number of nucleotides that may be divided by three, which starts with a start codon (e.g., ATG) and which preferably terminates with a stop codon (e.g., TAA, TGA, or TAG). The open reading frame may be isolated or it may be incorporated in a longer nucleic acid sequence, for example in a ceDNA vector as described herein.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present disclosure, is provided. As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. According to some aspects, the adults are seniors about 65 years or older, or about 60 years or older. According to some aspects, the subject is a pregnant woman or a woman intending to become pregnant. In other aspects, subject is not a human; for example a non-human primate; such as a baboon, a chimpanzee, a gorilla, or a macaque. In certain aspects, the subject may be a pet, such as a dog or a cat.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. According to some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleic acid sequence encoding a fusion variant polypeptide. Alternatively, the term “heterologous” may refer to a nucleic acid sequence which is not naturally present in a cell or subject.

A “vector” or “expression vector” is a replicon, such as plasmid, bacmid, phage, virus, virion, or cosmid, to which another DNA segment, i.e., an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. According to some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, according to some embodiments, be combined with other suitable compositions and therapies. According to some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms, “administration,” “administering” and variants thereof refers to introducing a composition or agent (e.g., a ceDNA as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, administration of a composition “subsequently to” administration of a composition indicates that a time interval has elapsed between administration of a first composition and administration of a second composition, regardless of whether the first and second compositions are the same or different.

The term “infection” as used herein refers to the initial entry of a pathogen into a host; and the condition in which the pathogen has become established in or on cells or tissues of a host; such a condition does not necessarily constitute or lead to a disease.

As used herein, the term “biological sample” refers to any type of material of biological origin isolated from a subject, including, for example, DNA, RNA, lipids, carbohydrates, and protein. The term “biological sample” includes tissues, cells and biological fluids isolated from a subject. Biological samples include, e.g., but are not limited to, whole blood, plasma, serum, semen, saliva, tears, urine, fecal material, sweat, buccal, skin, cerebrospinal fluid, bone marrow, bile, hair, muscle biopsy, organ tissue or other material of biological origin known by those of ordinary skill in the art. Biological samples can be obtained from subjects for diagnosis or research or can be obtained from healthy subjects, as controls or for basic research. The term “dose” as used herein refers to the quantity of a substance (e.g., a ceDNA as described herein) to be taken or administered to the subject at one time.

The term “dosing”, as used herein, refers to the administration of a substance (e.g., a ceDNA as described herein) to achieve a therapeutic objective (e.g., treatment).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present disclosure, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an antigen, or immunogenic protein, as described herein, and another drug. The other drug(s) may be administered concomitant with, prior to, or following the administration of the antigen, or immunogenic protein, as described herein.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA) or guide RNA (gRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). According to some embodiments, the therapeutic nucleic acid is a ceDNA.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, “viral infection” is meant to refer to the invasion and multiplication of a virus in the body of a subject.

The term “treatment” as used herein is meant to refer to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection). Treating may further refer to accomplishing one or more of the following: (a) reducing the severity of the disorder; ((b) limiting worsening of symptoms characteristic of the disorder(s) being treated; (c) limiting recurrence of the disorder(s) in patients that have previously had the disorder(s); and (d) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

The term “vaccinated” as used herein is meant to refer to being treated with a vaccine.

The term “vaccination” as used herein is meant to refer to treatment with a vaccine.

The term “vaccine” as used herein is meant to refer to a formulation which is in a form that is capable of being administered to a vertebrate and which induces a protective immune response sufficient to induce immunity and/or to prevent and/or ameliorate an infection and/or to reduce at least one symptom of an infection and/or to enhance the efficacy of another dose of a formulation.

Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat a viral infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The term “vaccine therapy” as used herein is meant to refer to a type of treatment that uses a substance or group of substances to stimulate the immune system to destroy a tumor or infectious microorganisms.

Those “in need of treatment” include mammals, such as humans, already having a disease or disorder, an infection, or a cancer.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, a “control” is meant to refer to a reference standard. According to some embodiments, the control is a negative control sample obtained from a healthy patient. In other embodiments, the control is a positive control sample obtained from a patient diagnosed with a disease or disorder, an infection or a cancer. In still other embodiments, the control is a historical control or standard reference value or range of values (such as a previously tested control sample, or group of samples that represent baseline or normal values). A difference between a test sample and a control can be an increase or conversely a decrease. The difference can be a qualitative difference or a quantitative difference, for example a statistically significant difference. According to some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±b 1%. The present disclosure is further explained in detail by the following examples, but the scope of the disclosure should not be limited thereto.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Other terms are defined herein within the description of the various aspects of the disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### II. Cells of the Immune System

There are a large number of cellular interactions that comprise the immune system. These interactions occur through specific receptor-ligand pairs that signal in both directions so that each cell receives instructions based on the temporal and spatial distribution of those signals.

Murine models have been highly useful in discovering immunomodulatory pathways, but clinical utility of these pathways does not always translate from an inbred mouse strain to an outbred human population, since an outbred human population may have individuals that rely to varying extents on individual immunomodulatory pathways.

Cells of the immune system include lymphocytes, monocytes/macrophages, dendritic cells, the closely related Langerhans cells, natural killer (NK) cells, mast cells, basophils, and other members of the myeloid lineage of cells. In addition, a series of specialized epithelial and stromal cells provide the anatomic environment in which immunity occurs, often by secreting critical factors that regulate growth and/or gene activation in cells of the immune system, which also play direct roles in the induction and effector phases of the response. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The cells of the immune system are found in peripheral organized tissues, such as the spleen, lymph nodes, Peyer's patches of the intestine and tonsils. Lymphocytes also are found in the central lymphoid organs, the thymus, and bone marrow where they undergo developmental steps that equip them to mediate the myriad responses of the mature immune system. A substantial portion of lymphocytes and macrophages comprise a recirculating pool of cells found in the blood and lymph, providing the means to deliver immunocompetent cells to sites where they are needed and to allow immunity that is generated locally to become generalized. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g., to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Two broad classes of lymphocytes are recognized: the B lymphocytes (B cells), which are precursors of antibody-secreting cells, and T lymphocytes (T cells).

### B Lymphocytes

B lymphocytes are derived from hematopoietic cells of the bone marrow. A mature B cell can be activated with an antigen that expresses epitopes that are recognized by its cell surface. The activation process may be direct, dependent on cross-linkage of membrane Ig molecules by the antigen (cross-linkage-dependent B cell activation), or indirect, via interaction with a helper T cell, in a process referred to as cognate help. In many physiological situations, receptor cross-linkage stimuli and cognate help synergize to yield more vigorous B cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B cell activation requires that the antigen express multiple copies of the epitope complementary to the binding site of the cell surface receptors, because each B cell expresses Ig molecules with identical variable regions. Such a requirement is fulfilled by other antigens with repetitive epitopes, such as capsular polysaccharides of microorganisms or viral envelope proteins. Cross-linkage-dependent B cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B cells to mount responses against antigens that cannot cross-link receptors and, at the same time, provides costimulatory signals that rescue B cells from inactivation when they are stimulated by weak cross-linkage events. Cognate help is dependent on the binding of antigen by the B cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T cells designated as CD4⁺ T cells. The CD4⁺ T cells bear receptors on their surface specific for the B cell's class II/peptide complex. B cell activation depends not only on the binding of the T cell through its T cell receptor (TCR), but this interaction also allows an activation ligand on the T cell (CD40 ligand) to bind to its receptor on the B cell (CD40) signaling B cell activation. In addition, T helper cells secrete several cytokines that regulate the growth and differentiation of the stimulated B cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4⁺ T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

### T Lymphocytes

T lymphocytes derived from precursors in hematopoietic tissue, undergo differentiation in the thymus, and are then seeded to peripheral lymphoid tissue and to the recirculating pool of lymphocytes. T lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B cell receptors see epitopes expressed on the surface of native molecules. While antibody and B cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4⁺ T cells); and those that express CD8 (CD8⁺ T cells). These cells differ in how they recognize antigen and in their effector and regulatory functions.

CD4⁺ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8⁺ T cells, can develop into cytotoxic T lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4⁺ T cells recognize only peptide/class II complexes while CD8⁺ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4⁺ T cells with TCRs capable of recognizing the expressed cell surface complex. Thus, CD4⁺ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8⁺ T cells expressing appropriate receptors. This gives the T cell system, particularly CD8⁺ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B cell surface membrane. T cells with receptors specific for the peptide/class II molecular complex recognize this complex on the B cell surface. (Paul, W. E., “Chapter 1: The immune system: an introduction,” Fundamental Immunology, 4th Edition, Ed. Paul, W. E., Lippicott-Raven Publishers, Philadelphia (1999)).

B cell activation depends both on the binding of the T cell through its TCR and on the interaction of the T cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B cell activation. The subsequent events in the B cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4⁺ T cells tend to differentiate into cells that principally secrete the cytokines IL-4, IL-5, IL-6, and IL-10 (TH2 cells) or into cells that mainly produce IL-2, IFN-γ, and lymphotoxin (TH1 cells). The TH2 cells are very effective in helping B cells develop into antibody-producing cells, whereas the TH1 cells are effective inducers of cellular immune responses, involving enhancement of microbicidal activity of monocytes and macrophages, and consequent increased efficiency in lysing microorganisms in intracellular vesicular compartments. Although CD4⁺ T cells with the phenotype of TH2 cells (i.e., IL-4, IL-5, IL-6 and IL-10) are efficient helper cells, TH1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

### T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. TH1 cells are effective in enhancing the microbicidal action, because they produce IFN-γ. In contrast, two of the major cytokines produced by TH2 cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Schwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4⁺ T (Treg) cells (Reviewed in Kronenberg, M. et al., “Regulation of immunity by self-reactive T cells”, Nature, Vol. 435: 598-604 (2005)). CD4⁺ Tregs that constitutively express the IL-2 receptor alpha (IL-2Ra) chain (CD4⁺CD25⁺) are a naturally occurring T cell subset that are anergic and suppressive (Taams, L. S. et al., “Human anergic/suppressive CD4⁺CD25⁺ T cells: a highly differentiated and apoptosis-prone population”, Eur. J. Immunol. Vol. 31:1122-1131 (2001)). Depletion of CD4⁺CD25⁺ Tregs results in systemic autoimmune disease in mice. Furthermore, transfer of these Tregs prevents development of autoimmune disease. Human CD4⁺CD25⁺ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4⁺CD25⁺ T cells can be split into suppressive (CD25^{high}) and nonsuppressive (CD25^{low}) cells, according to the level of CD25 expression. A member of the forkhead family of transcription factors, FOXP3, has been shown to be expressed in murine and human CD4⁺CD25⁺ Tregs and appears to be a master gene controlling CD4⁺CD25⁺ Treg development (Battaglia, M. et al., “Rapamycin promotes expansion of functional CD4⁺CD25⁺Foxp3⁺ regulator T cells of both healthy subjects and type 1 diabetic patients”, J. Immunol., Vol. 177: 8338-8347, (2006)).

### Cytotoxic T Lymphocytes

CD8⁺ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

### T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269rv1, (2015)). CD45RA is expressed on naïve T cells, as well as the effector cells in both CD4 and CD8. After antigen experience, central and effector memory T cells gain expression of CD45RO and lose expression of CD45RA. Thus either CD45RA or CD45RO is used to generally differentiate the naïve from memory populations. CCR7 and CD62L are two other markers that can be used to distinguish central and effector memory T cells. Naïve and central memory cells express CCR7 and CD62L in order to migrate to secondary lymphoid organs. Thus, naïve T cells are CD45RA⁺CD45RO⁻CCR7⁺CD62L⁺, central memory T cells are CD45RA⁻CD45RO⁺CCR7⁺CD62L⁺, and effector memory T cells are CD45RA⁻CD45RO⁺CCR7⁻CD62L⁻.

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

### III. Expression of Peptides from a DNA Vector

Provided herein are methods of inducing an immune response against a first peptide and a second peptide in a subject, comprising administering a priming vaccine comprising a deoxyribonucleic acid (DNA) DNA to the subject, wherein the DNA encodes a first peptide; and administering a boosting vaccine comprising (i) a ribonucleic acid (RNA), or (ii) a second peptide to the subject, wherein the RNA encodes the second peptide, thereby inducing the immune response against the first peptide and the second peptide in the subject.

Also provided are vaccine regimens, comprising a priming vaccine comprising a deoxyribonucleic acid (DNA) DNA, wherein the DNA encodes a first peptide; and a boosting vaccine comprising (i) a ribonucleic acid (RNA), or (ii) a second peptide, wherein the RNA encodes the second peptide.

According to some embodiments, the priming vaccine comprises DNA in the form of a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector. According to some embodiments, the priming vaccine comprises DNA in the form of a plasmid. According to some embodiments, the priming vaccine comprises DNA in the form of ceDNA.

According to some embodiments, the priming vaccine comprises DNA in the form of a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector and the boosting vaccine comprises an RNA (e.g., mRNA).

According to some embodiments, the priming vaccine comprises DNA in the form of a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector and the boosting vaccine comprises a peptide.

### DNA Plasmids

According to some embodiments, the priming vaccine comprises DNA in the form of a DNA plasmid comprising a nucleic acid sequence encoding a selected antigen to which an immune response is desired. In the plasmid, the selected antigen is under the control of regulatory sequences directing expression thereof in a mammalian or vertebrate cell.

The components of the plasmid itself are known in the art.

Non-viral, plasmid vectors useful in this invention contain isolated and purified DNA sequences comprising DNA sequences that encode a selected antigen, e.g., an antigen described herein. The DNA molecule may be derived from viral or non-viral, e.g., bacterial species that have been designed to encode an exogenous or heterologous nucleic acid sequence. Such plasmids or vectors can include sequences from viruses or phages. A variety of non-viral vectors are known in the art and may include, without limitation, plasmids, bacterial vectors, bacteriophage vectors, “naked” DNA and DNA condensed with cationic lipids or polymers.

Examples of bacterial vectors include, but are not limited to, sequences derived from bacille Calmette Guerin (BCG), Salmonella, Shigella, E. coli, and Listeria, among others. Suitable plasmid vectors include, for example, pBR322, pBR325, pACYC177, pACYC184, pUC8, pUC9, pUC18, pUC19, μLG339, pR290, pK37, pKC101, pAC105, pVA51, pKH47, pUB110, pMB9, pBR325, Col E1, pSC101, pBR313, pML21, RSF2124, pCR1, RP4, pBAD18, and pBR328.

Examples of suitable inducible Escherichia coli expression vectors include pTrc (Amann et al., 1988 Gene, 69:301-315), the arabinose expression vectors (e.g., pBAD18, Guzman et al., 1995 J. Bacteriol., 177:4121-4130), and pETIId (Studier et al., 1990 Methods in Enzymology, 85:60-89).

The promoter and other regulatory sequences that drive expression of the antigen in the desired mammalian or vertebrate host may similarly be selected from a wide list of promoters known to be useful for that purpose. A variety of such promoters are disclosed below. Exemplary promoters include, but are not limited to, the human cytomegalovirus (HCMV) promoter/enhancer (described in, e.g., U.S. Pat. Nos. 5,168,062 and 5,385,839, and the SCMV promoter enhancer.

Additional regulatory sequences for inclusion in a nucleic acid sequence, molecule or vector include, without limitation, an enhancer sequence, a polyadenylation sequence, a splice donor sequence and a splice acceptor sequence, a site for transcription initiation and termination positioned at the beginning and end, respectively, of the polypeptide to be translated, a ribosome binding site for translation in the transcribed region, an epitope tag, a nuclear localization sequence, an IRES element, a Goldberg-Hogness “TATA” element, a restriction enzyme cleavage site, a selectable marker and the like. Enhancer sequences include, e.g., the 72 bp tandem repeat of SV40 DNA or the retroviral long terminal repeats or LTRs, etc. and are employed to increase transcriptional efficiency.

These other components useful in DNA plasmids, including, e.g., origins of replication, polyadenylation sequences (e.g., BGH polyA, SV40 polyA), drug resistance markers (e.g., kanamycin resistance), and the like may also be selected from among sequences well known in the art.

Selection of promoters and other common vector elements are conventional and many such sequences are available with which to design the plasmids useful in this invention. See, e.g., Sambrook et al, Molecular Cloning. A Laboratory Manual, Cold Spring Harbor Laboratory, New York, (1989) and references cited therein at, for example, pages 3.18-3.26 and 16.17-16.27 and Ausubel et al., Current Protocols in Molecular Biology, John Wiley & Sons, New York (1989). All components of the plasmids may be readily selected by one of skill in the art from among known materials in the art and available from the pharmaceutical industry.

Examples of suitable DNA plasmid constructs that may be used in the priming vaccines described herein are set forth in detail in the following patent publications, which are International Patent Publication Nos. WO98/17799, WO99/43839 and WO98/17799; and U.S. Pat. Nos. 5,593,972; 5,817,637; 5,830,876; and 5,891,505, incorporated by reference in their entireties herein.

ceDNA vectors

According to some embodiments, the technology described herein is directed in general to the expression and/or production of an antigen in a cell from one or more non-viral DNA vectors, e.g., ceDNA vectors as described herein. ceDNA vectors for expression of an antigen are described herein in the section entitled “ceDNA vectors in general”. As previously discussed, a distinct advantage of ceDNA vectors over traditional AAV vectors, and even lentiviral vectors, is that there is no size constraint for the one or more nucleic acid sequences that encode a peptide (e.g., an antigen). The skilled artisan would appreciate, based upon the disclosure provided herein, that numerous peptide antigens can be used to produce an almost limitless variety of ceDNA vectors once armed with the teachings provided herein.

In some embodiments, ceDNA vectors for expression of a peptide (e.g., an antigen), comprise a pair of ITRs (e.g., symmetric or asymmetric as described herein) and between the ITR pair, a nucleic acid encoding an antigen, or an immunogenic peptide, as described herein, operatively linked to a promoter or regulatory sequence. A distinct advantage of ceDNA vectors for expression of an antigen, or an immunogenic peptide, over traditional AAV vectors, and even lentiviral vectors, is that there is no size constraint for the nucleic acid sequences encoding the desired antigen, or immunogenic peptide.

As one will appreciate, the ceDNA vector technologies described herein can be adapted to any level of complexity or can be used in a modular fashion, where expression of different components of the ceDNA vector can be controlled in an independent manner. The following embodiments are specifically contemplated herein and can adapted by one of skill in the art as desired.

According to some aspects, the present disclosure provides one or more ceDNA vectors comprising one or more nucleic acid sequences that encode an antigen. According to some embodiments, the one or more nucleic acid sequences encode one or more peptides (e.g., antigens) from a variety of pathogens, including, e.g., bacterial, viral, fungal and parasitic infectious agents. According to some embodiments, the one or more nucleic acid sequences encode one or more peptides (e.g., antigens) that are cancer or cancer-associated antigens. According to some embodiments, the antigen or immunogenic peptide is a tumor antigen. According to some embodiments, the one or more nucleic acid sequences encode one or more peptides (e.g., antigens) that are associated with an autoimmune condition, such as rheumatoid arthritis (RA) or multiple sclerosis (MS). According to some embodiments, the antigen is an antigen relating to an autoimmune disorder or condition, such as an autoimmune disease triggered by an infectious agent, or to an infectious disease or pathogen.

### Cancer or Tumor-Associated Antigens

According to some embodiments, the ceDNA comprises a nucleic acid sequence that encodes is a cancer or a tumor-associated antigen. According to some embodiments, the ceDNA comprises a nucleic acid sequence that encodes one or more antigens selected from the Cancer Antigenic Peptide Database, publicly available at caped.icp.ucl.ac.be/about. This database includes the peptide sequence and its position in the protein sequence, for each antigen identified.

According to some embodiments, the ceDNA comprises a nucleic acid sequence that encodes a tumor-associated antigen selected from one of more of the antigens set forth in Table 1 below:

TABLE 1

| | | |
|---|---|---|
| Mesothelin | alpha fetal protein (AFP) | kRAS |
| NY-ESO-1 | cancer embryo antigen | Ep-CAM |
| | (CEA) | |
| FBP | SOX2 | MUC1 |
| HER-2/neu | GOLGA | Survivin |
| IL-13 receptor α2 | TPR | hTERT |
| melanoma-related antigens- | U2AF1L | WT1 |
| MAGE-1, MAGE-2, MAGE-3 | | |
| EphA2 | CYNL2 | C13orf53 |
| P53 | NSEP1 | RBPSUH |
| NKTR | C13orf24 | IG4 |
| GLEA2 | TNKS2 | HSPH1 |
| BRAP | RTN4 | KIAA0376 |
| SART1 | C9orf112 | AIM-3 |
| IL-13R alpha | TRP-2 | HER-2 |
| TRP-1 | ras | PSMA |
| PSA | PAP | MUM-1 |
| MART-1 | gp100 | gp75 |
| tyrosinase (Tyr) | midkin (MK) | BAGE |
| CASP-8 | β-catenin | CA-125 |
| CDK-1 | ESO-1 | |

Recent analyses of The Cancer Genome Atlas (TCGA) datasets have linked the genomic landscape of tumors with tumor immunity, implicating neoantigen load in driving T cell responses (Brown et al., Genome Res. 2014 May; 24(5):743-50, 2014) and identifying somatic mutations associated with immune infiltrates (Rutledge et al., Clin Cancer Res. 2013 Sep. 15; 19(18):4951-60, 2013). Rooney et al. (2015 Jan. 15; 160(1-2):48-61) suggest that neoantigens and viruses are likely to drive cytolytic activity, and reveal known and novel mutations that enable tumors to resist immune attack.

In some embodiments, the antigen is a neoantigen identified from a cancer cell in a subject. In some embodiments, the neoantigen is a shared neoantigen. Methods of identifying neoantigens are known in the art and described, e.g., in U.S. Pat. No. 10,055,540, incorporated by reference in its entirety herein. Neoantigenic polypeptides and shared neoantigenic polypeptides are described, for example, in PCT/US2016/033452, U.S. Publication No. 20180055922, Schumacher and Hacohen et al. (Curr Opin Immunol. 2016 August; 41:98-103), Gubin, M M et al. (Nature. 2014 Nov. 27; 515(7528):577-81), Schumacher and Schreiber, Science. 2015 Apr. 3; 348(6230):69-74), Ott PA., et al., Nature. 2017 Jul. 13; 547(7662):217-221, all of which are incorporated by reference in their entireties herein.

Accordingly, in some embodiments, the antigen is a neoantigen polypeptide. In some embodiments, the antigen is a neoantigen polypeptide set forth in The Comprehensive Tumor-Specific Neoantigen Database (TSNAdb v1.0); available at biopharm.zju.edu.cn/tsnadb and described in Wu et al., Genomics Proteomics Bioinformatics 16 (2018) 276-282. In some embodiments, the antigen is a neoantigen polypeptide set forth in U.S. Pat. No. 10,055,540, incorporated by reference in its entirety herein.

### Autoimmune Disease Antigens

According to some embodiments, antigen is associated with an autoimmune disease. According to some embodiments, the ceDNA comprises a nucleic acid sequence that encodes one or more antigens selected from those in Table 2, below.

TABLE 2

| Disease | Antigen |
|---|---|
| Neuromyelitis Optica (NMO) | Aquaporin 4 (AQP4) |
| Myasthenia Gravis (MG) | Acetylcholine receptor (AchR) |
| Membranous glomerulonephritis | Phospholipase A2 receptor (PLA2R) |
| Pemphigus Vulgaris (PV) | desmoglein 3 (DSG3) |
| Pemphigus Foliaceus (PF) | desmoglein 1 (DSG1) |
| Type I diabetes mellitus (T1DM) | Insulin/proinsulin/preproinsulin |
| Type I diabetes mellitus (T1DM) | glutamate decarboxylase (GAD65) |
| Type I diabetes mellitus (T1DM) | insulinoma antigen-2 (IA-2) |
| Multiple Sclerosis (MS) | myelin oligodendrocyte glycoprotein (MOG) |
| Multiple Sclerosis (MS) | myelin basic protein (MBP) |
| Multiple Sclerosis (MS) | proteolipid protein (PLP) |
| anti-phospholipid syndrome (APS)/ CAPS | beta-2 glycoprotein 1 (b2GP1) |
| celiac disease | A-gliadin |
| Acute rheumatic fever | cross reactive antibodies to cardiac muscle |
| alopecia areata | Trychohyalin, keratin 16 |
| ANCA-associated vasculitis | Neutrophil cytoplasmic antigen, proteinase 3, |
| | myeloperodixase, bacterial permiability |
| | increasing factor |
| autoimmune gastritis | H, K adenosine triphosphatase |
| Autoimmune hemolytic | Rh blood group antigens, 1 antigen |
| anemia | |
| autoimmune hepatitis | nuclear protein, liver-kidney microsome type 1, |
| | liver cytosol type 1 |
| autoimmune myocarditis | cardiac myosin |
| Autoimmune thyroiditis | Thyroid peroxidase, thyroglobulin, thyroid- |
| | stimulating hormone receptor |
| Autoimmune uveitis | Retinal arrestin (S-antigen) |
| dermatomyositis | Mi2 ATPase |
| diabetes (type 1) | Pancreatic beta cell antigen |
| good pasture's syndrome | Noncollagenous domain of basement membrane |
| | collagen type IV |
| Graves' disease | Thyroid stimulating hormone receptor |
| Guillain-Barré syndrome | Neurofascin-18G, gliomedin, nodal adhesion |
| | molecueles |
| Hypoglycemia | Insulin receptor |
| Idiopathic thrombocytopenia purpura | Platelet integrin Gpllb, Gpllla |
| Insulin resistant diabetes | Insulin receptor |
| Membranous nephritis | Phospholipase AZ |
| mixed essential cryoglobulinemia | rheumatoid factor IgG complexes |
| multiple sclerosis | Myelin basic protein, proteolipid protein, |
| | myelin oligodendrocyte glycoprotein |
| myasthenia gravis | Acetylcholine receptor |
| Myasthenia gravis-MUSC | Muscarinic receptor |
| pemphigus/pemphigoid | Epidermal cadherin |
| pernicious anemia | intrinsic factor (Gastric) |
| polymyositis | nuclear and nucleolar antigen |
| primary biliary cirrhosis | neutrophil nuclear antigen, mitochondrial |
| | multienzyme complex |
| psoriasis | PSO p27 |
| rheumatoid arthritis | rheumatoid factor IgG complexes, synovial joint |
| | antigen, citrullinated protein, carbamylated |
| | protein |
| scleroderma/systemic sclerosis | Scl-86, nucleolar scleroderma antigen |
| Sjogren's syndrome | SS-B, Lupus La protein |
| systemic lupus erythematosus | DNAr histones, ribosomes, snRNP, scRNP |
| vitiligo | VIT-90, VIT-75, VIT-40 |
| Wegener's granulomatosis | neutrophil nuclear antigen |
| Antiphospholipid syndrome (APS) & | Beta-2 glycoprotein 1 |
| catastrophic APS | |
| Chemotherapy induced peripheral neuropathy | Neuronal antigens |
| Atypical hemolytic uremic syndrome | Complement factor H |
| Thrombotic thrombocytopenia purpura | ADAMTS13 |

According to some embodiments, the autoimmune disease is triggered by an infectious agent. According to some embodiments, the disclosure provides a ceDNA as described herein comprising a nucleic acid sequence that encodes one or more peptides (e.g., antigens) for treating an autoimmune disease or disorder associated with or triggered by an infectious agent. Exemplary autoimmune diseases or disorders associated with or triggered by infectious agents are provided in Table 3.

TABLE 3

| Autoimmune Disease | Infectious Agent(s) |
|---|---|
| Allergic encephalitis | Measles virus |
| Autoimmune kidney disease | Streptococcal infections |
| Chagas disease | Trypanosoma cruzi |
| Chronic autoimmune hepatitis | Hepatitis C virus |
| Guillain-Barré syndrome | Campylobacter jejuni, Cytomegalovirus, Zika virus |
| Herpetic stromal keratitis | Herpes simplex virus |
| HTLV-associated myelopathy | Human T-cell leukemia virus |
| Lyme arthritis | Borrelia burgdorferi |
| Mixed cryoglobulinemia | Hepatitis C virus |
| Myocarditis | Coxsackie virus B3 |
| Pediatric autoimmune neuropsychiatric | Streptococcal infections |
| disorders | |
| Polyarteritis nodosa | Hepatitis B virus |
| Primary biliary cirrhosis | Escherichia coli |
| Reactive arthritis | Yersinia enterocolitica |
| Reiter's syndrome | Chlamydia trachomatis, Shigella species |
| Rheumatic fever | Streptococcus pyogenes |
| Rheumatic heart disease | Streptococci |
| Rheumatoid arthritis | Normal gut flora |
| Scleroderma | Cytomegalovirus |
| Tourette syndrome | Streptococcal infections |
| Type 1 diabetes | Enterovirus, Rotavirus |
| Type 1 diabetes mellitus | Coxsackie virus B4 |

### Infectious Diseases

According to some embodiments, the disclosure provides a ceDNA as described herein comprising a nucleic acid sequence that encodes one or more peptides (e.g., antigens) for treating an infectious disease. According to some embodiments, the antigen is an antigen of a pathogen or infectious agent (where “pathogen” and “infectious agent” are used interchangeably herein), e.g., a viral pathogen, a bacterial pathogen, a fungal pathogen, or a parasitic pathogen.

According to some embodiments, the antigen is a viral antigen. According to some embodiments, the disclosure provides a ceDNA as described herein comprising a nucleic acid sequence that encodes one or more viral antigens.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Exemplary viral pathogens are shown below in Table 4.

TABLE 4

| | Examples, by | |
|---|---|---|
| | Family (Human | |
| Baltimore classification | Host) | Species / Pathology example |
| dsDNA viruses | Adenoviridae | Example Respiratory infection |
| dsDNA viruses | Polyomaviridae | Example - progressive multifocal |
| | | leukoencephalopathy |
| dsDNA viruses | Papiliomaviradae | Example - betapapilloma virus (warts, malignant |
| | | tumors) |
| dsDNA viruses | Poxviridae | Example - Molluscum contagiosum (skin lesions) |
| dsDNA viruses | Herpesvirales | Example - Varicellovirus (Chickenpox) |
| ssDNA viruses (+strand | Anelloviridae | Asymptomatic, may be associate with hepatitis, |
| or “sense”) DNA | | pulmonary disease, hematological disorders, |
| | | myopathy, lupus |
| ssDNA viruses (+strand | Parvoviridae | Example - Fifth disease |
| or “sense”) DNA | | |
| dsRNA | Reoviridae | Example - Colorado tick fever |
| viruses (e.g., Reoviruses) | | |
| (+)ssRNA viruses | Coronaviridae | Example - Pneumonia, gastroenteritis |
| (+strand or sense) RNA | | |
| (+)ssRNA viruses | Picornaviridae | Example - Myocarditis |
| (+strand or sense) RNA | | |
| (+)ssRNA viruses | Astoviridae | Example - infantile gastroenteritis |
| (+strand or sense) RNA | | |
| (+)ssRNA viruses | Caliciviridae | Example - Norovirus - gastroenteritis |
| (+strand or sense) RNA | | |
| (+)ssRNA viruses | Flaviviridae | Example - Dengue, Zika |
| (+strand or sense) RNA | | |
| (+)ssRNA viruses | Hepeviridae | Example - Hepatits |
| (+strand or sense) RNA | | |
| (+)ssRNA viruses | Togaviridae | Example - Rubella |
| (+strand or sense) RNA | | |
| (−)ssRNA viruses | Rhabdoviridae | Example - Rabies |
| (−strand or antisense) RNA | | |
| (−)ssRNA viruses | Filoviridae | Example - Ebola, Marburg |
| (−strand or antisense) RNA | | |
| (−)ssRNA viruses | Paramyxoviridae | Example - Mumps |
| (−strand or antisense) RNA | | |
| (−)ssRNA viruses | Pneumovirinae | Example - Respiratory tract infection |
| (−strand or antisense) RNA | | |
| (−)ssRNA viruses | Arenaviridae | Example - Enchephalitis, Hemorrhagic fever |
| (−strand or antisense) RNA | | |
| (−)ssRNA viruses | Bunyaviridae | Example - Hantavirus pulmonary |
| (−strand or antisense) RNA | | syndrome/hemorrhagic fever |
| (−)ssRNA viruses | Deltavirus | Example - hepatitis, cirrhosis |
| (−strand or antisense) RNA | | |
| (−)ssRNA viruses | Orthomyxoviridae | Example - Influenza A, Influenza B |
| (−strand or antisense) RNA | | |
| ssRNA-RT viruses | Retroviridae | Example - lentivirus - HIV |
| (+strand or sense) RNA with | | |
| DNA intermediate in life- | | |
| cycle | | |
| dsDNA-RT viruses | Hepadnaviridae | Example disease - hepatitis, cirrhosis, hepatocellular |
| | | carcinoma |

According to some embodiments, the disclosure provides a ceDNA as described herein comprising a nucleic acid sequence that encodes one or more peptides (e.g., antigens) for treating COVID-19. According to some embodiments, the nucleic acid encodes the SARS-CoV-2 spike protein.

The spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion.

The complete genome of severe acute respiratory syndrome coronavirus 2 isolate Wuhan-Hu-1 is set forth as GenBank Accession No. MN908947.3. The amino acid sequence of the wild type spike glycoprotein (S), is set forth below as SEQ ID NO:__:

MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

According to some embodiments, the peptide, is the stabilized prefusion SARS-CoV-2 spike protein (SARS-CoV-2 S(2P)).

According to some embodiments, peptide, is a bacterial antigen. According to some embodiments, the disclosure provides a ceDNA as described herein comprising a nucleic acid sequence that encodes one or more bacterial antigens.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella bumetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

Exemplary bacterial infections are shown in Table 5 below.

TABLE 5

| Phylum | By Genus | Species / Pathology Example |
|---|---|---|
| Proteobacteria | Erlichia | Erlichosis (Pain, malaise, fever gastrointestinal disorder, |
| | | confusion rash. Can be fatal) |
| Proteobacteria | Rickettsia | Two groups of diseases from different subspecies - Spotted |
| | | fever; Typhus |
| Proteobacteria | Brucella | Brucellosis. 4 known species cause disease in humans. Can be |
| | | sporadic or chronic. Fever, malaise, lesions |
| Proteobacteria | Bartonella | Multiple species. Pathophysiology includes endocarditis, |
| | | neuroretinitis, cat scratch disease, peliosis hepititus, general |
| | | bactermia angiomatosis, Carrion's disease, Trench fever |
| Proteobacteria | Bordetella | Multiple species. Adherence to ciliated ephilium drives |
| | | respiratory tract infections. B. pertussis = subspecies example |
| Proteobacteria | Neisseria | Includes N meningitidis (bacterial meningitis and septicemia) |
| | | and N gonorrheae (Gonorrhea) - colonize mucosal surfaces |
| Proteobacteria | Francisella | Examples include f. tularensis (tularemia), F novicida, |
| | | F philomiragia (septicemia) |
| Proteobacteria | Legionella | Examples include L pneumophila - legionnaires disease and |
| | | pontiac fever |
| Proteobacteria | Coxiella | Coxiella burnetii - Q Fever |
| Proteobacteria | Moraxella | Examples include M. catarrhalis (lower respiratory tract |
| | | infection) and Moraxella lacunata (blepharoconjunctivitis) |
| Proteobacteria | Pseudomonas | Multiple groups and subspecies therein. Examples include |
| | | P. aeruginosa - opportunistic infection in cystic fobrosis, burn and |
| | | immunocompromised patients. Colonizes skin, lungs, kidney, |
| | | urinary tract. Found on most medical equipment - develops |
| | | enduring biofilms |
| Proteobacteria | Vibrio | Multiple species - foodborne disease generally implicated with |
| | | gastroenteritis - example - V. cholerae. Can also cause |
| | | septicemia in open wounds |
| Proteobacteria | Plesimonas | Example P. Shigelloides - causes gastrointestinal disease |
| Proteobacteria | Aeromonas | Cornucopia of human disease including and not limited to |
| | | gastrointestinal disease, wound and soft tissue infection, blood |
| | | borne dyscrasias |
| Proteobacteria | Citrobacter | Examples: C. freundii, C. koseri, C. amalonaticus. Infect |
| | | urinary tract, can cause infant meningitis and sepsis |
| Proteobacteria | Enterobacter | Multiple species - Urinary and respiratory tract infections are |
| | | most common. Example E. aerogenes are a common source of |
| | | opportunistic and / or nosocomial infections |
| Proteobacteria | Escerichia | Common cause of gastrointestinal infections. Example E. coli |
| Proteobacteria | Klebsiella | Multiple species and sub-species. Cause a variety of |
| | | opporuntistic infections: Pneumonia, UTI, septicemia, |
| | | meningitis, diarrhea, soft tissue infection |
| Proteobacteria | Proteus | Urinary tract infections (including kidney) - P. vulgaris, |
| | | P. mirabilis, P. penneri |
| Proteobacteria | Providencia | Multiple species cause. Urinary tract infections. Example: |
| | | P. stuartii |
| Proteobacteria | Morganella | Single species with 2 subspecies. M. morganii. Cause |
| | | opportunistic infections (wound, UTI) |
| Proteobacteria | Salmonella | Two species S. bongori and S. enterica, along with multiple |
| | | subspecies. Typical non-typhoidal salmonella causes |
| | | gastrointestinal disease. In developing countries it also causes |
| | | blood infections. Typhiidal disease causes typhoid fever, |
| | | hypovolemic shock, septic shock |
| Proteobacteria | Serratia | Multiple species: Opportunisite infection that often develops |
| | | biofilms. Colonizes respiratory and urinary tract. Responsible |
| | | for 2% of nosocomial infections of blood, LRT, UT, surgical |
| | | wounds, skin and soft tissue infection. Example s. marcescens |
| Proteobacteria | Shigella | 4 species. Causes shigellosis (leading bacterial cause of |
| | | diarrhea). Example: S. dysenteriae |
| Proteobacteria | Yersinia | Multiple species and sub-species. Example: Y. pestis causes the |
| | | plague |
| Proteobacteria | Pasteurella | Multiple species. P. multocida species is the most frequent |
| | | example of human infection (-symptoms include swelling, |
| | | cellulitis, wound drainage and arthritis) |
| Proteobacteria | Hemophilus | Multiple species. Example H influenzae (Hib) causes |
| | | penumonia, sepsis and bacterial meningitis in young children |
| Proteobacteria | Campylobacter | Multiple species. Cause campylobacteriosis (gastrointestinal |
| | | disease - inflammatory diarrhea/dysentary). Example |
| | | C. rectus |
| Proteobacteria | Heliobacter | Example H pylori. Causes gastritis and gastric ulcers |
| Frimicutes | Clostridia | Multiple Species. Examples: C. Difficile, C. botulinum, |
| | | C. tetani. Cause a variety of serious conditions, from colitis to |
| | | paralysis |
| Frimicutes | Mycoplasmas | Multiple species. Examples: M. genitalium, M pneumoniae. |
| | | Ureaplasma species. Associated with sexually transmited |
| | | disease, infertility and infant respiratory distress and brain |
| | | hemorrhage. P1 antigen is primary virulence factor, which is |
| | | also expressed on erthyrocytes. This can lead to autoantibody |
| | | agglutination |
| Frimicutes | Bacillus | One of the most diverse genus from a speciation perspective. |
| | | Examples B. anthracis (anthrax), B. cereus (food poisoning) |
| Frimicutes | Listeria | 15 identified species. Example: L. monocytogenes food |
| | | poisining. Less frequently seen disease manifestation = |
| | | listeriosis (sepsis and meningitis with a 20% fatality rate) |
| Frimicutes | Staphylococcus | Multiple species and sub. Example: S. aureus. Disease can |
| | | range form folliculitis to necrotizing pneumonia and |
| | | endocarditis. Commonly drug resistant (MRSA) |
| Frimicutes | Enterococcus | Multiple species. Cause UTI, Bacteremia, endocarditis, |
| | | diverticulitis, meningitis, prostatitis. Example: E. faecium. |
| | | Increasingly drug resistant (VRE) |
| Frimicutes | Lactobacillus | Often considered beneficial, but can cause septicemiua, |
| | | endocarditis, rheumatic vascular disease and dental |
| | | caries(particularly in immuno-compromised patients). Example |
| | | L. rhamnosus |
| Frimicutes | Streptococcus | Multiple species. Cause strep throat, pink eye, meningitis, |
| | | bacterial pneumonia, sepsis, endocarditis, erysipelas, necrotizing |
| | | fasciitis. Examples: S. pyogenes, S. pneumoniae, S. sanguinis |
| Actinobacteria | Nocardia | Multiple species. Low virulence and generally infect the |
| | | immunocompromised only. Cause penumonia, endocarditis, |
| | | encephalitis, cellulitis. Example N. asteroids |
| Actinobacteria | Mycobacterium | Multiple species. Cause tuberculosis, leprosy. Examples |
| | | M. tuberculosis, M. leprae |
| Actinobacteria | Corynebacterium | Multiple species. Cause diptheria, colonizes prosthetics, and |
| | | can cause skin infections, endocarditis, pheumonitis. |
| | | Nosocomial |
| Actinobacteria | Actinomyces | Multiple species. Cause periodontal abscesses, |
| | | lympadenopathy, thoracic disease and abdominal abscess. |
| | | Example: Aggregatibacter actinomycetemcomitans |
| Chlamydia | Chlamydia | 4 species. Most common bacterial STD and can cause blindness. |
| | | Example Chlamydia trachomatis. |
| Spirochetes | Borrelia | 52 species. Cause Lyme disease and relapsing fever (severe |
| | | bacterimia). Example: Borrelia burgdorferi |
| Spirochetes | Leptospira | Multiple species (13 cause disease in humans). Cause |
| | | leptospirosis - symtpoms range from hedaches and fatigue to |
| | | meningitis, kidney failure and pulmonary hemorrhage. |
| Spirochetes | Treponema | Multiple species. Cuases syphilis Example: Treponema |
| | | pallidum. |
| Bacteroids | Bacteroides | Multiple species. Colonize the gut and cause infections |
| | | associated with surgery, appendicitis etc. Example: B. fragilis |

According to some embodiments, the antigen is a fungal antigen or immunogenic peptide. According to some embodiments, the disclosure provides a ceDNA as described herein comprising a nucleic acid sequence that encodes one or more fungal antigens.

Exemplary fungal infections are shown in Table 6 below.

TABLE 6

| Fungal | | |
|---|---|---|
| Group | Pathogen Species | Pathology example |
| Yeast | Candida albicans | Oral thrush, onychomycosis |
| Yeast | Candida glabrata | Vaginitis, esophageal candidisis |
| Yeast | Candida krusei | Invasive candidiasis, neutropenia |
| Yeast | Candida | Catheter and central line infections, biofims, UTI, endocarditis, |
| | parapsilosis | meningitis |
| Yeast | Candida tropicalis | Candidemia, oral thrush |
| Yeast | Rhodotorula | Sepsis, catheter infections, fungemia, meningitis, peritonitis |
| | mucilaginosa | |
| Yeast | Sporothrix schenckii | Sporotrichosis (lympocutaneous tissue, skin ulcerations) |
| | complex | |
| Yeast | Cryptococcus | Cryptococcal meningitis, pulmonary infection, osteomyelitis |
| | neoformans | |
| Yeast | Cryptococcus gattii | Cryptococcal meningitis, pulmonary infection, osteomyelitis (more |
| | | common in immunocompromised patients thant C. neoformans, |
| | | which is more prevelant in immunocompetent patients) |
| Molds | Alternaria alternata | Allergic fungal rhinosinusitis, Keratitis, peritonitis, allergic |
| | | respiratory disease |
| Molds | Apophysomyces | Cutaneous and subcutaneous infection |
| | variabilis | |
| Molds | Aspergillus | Chronic pulmonary aspergillosis, Asthma exacerbation with fingal |
| | fumigatus | sensitization, chronic invasive sinusitis, invasive and disseminated |
| | | aspergillosis |
| Molds | Aspergillus flavus | Chronic cavity pulmonary aspergillosis, Cutaneous and wound |
| | | infection, endocarditis, pericarditis and CNS infection, UTI |
| Molds | Aspergillus niger | Otomycosis, SAFS, Allergic bronchopulmonary aspergillosis, |
| | | invasive pulmonary aspergillosis, disseminated aspergillosis |
| Molds | Aspergillus terreus | ABPA, aspergillus bronchitis, invasive aspergillosis, disseminated |
| | | aspergillosis |
| Molds | Cladosphialophora | Chromoblastomycosis, mycetoma, phaehyphomycosis |
| | spp. | |
| Molds | Exserohilum | Skin, corneal infection, invasive infection of sinus heart and lung |
| Molds | Fonsecaea pedrosoi | Chromoblastomycosis |
| Molds | Fusarium | Keratitis, Onychomycosis, endophthalmitis, skin infection, |
| | oxysporum | sinusitis, disseminated infection |
| Molds | Fusarium solani | Keratitis, Onychomycosis, endophthalmitis, skin infection, |
| | | sinusitis, disseminated infection |
| Molds | Lichtheimia | cutaneous, pulmonary, rhinocerebral CNS and disseminated |
| | corymbifera | infection (rare) |
| Molds | Lichtheimia ramosa | cutaneous, pulmonary, rhinocerebral CNS and disseminated |
| | | infection (rare) |
| Molds | Rhizopus | Mucormycosis |
| | microsporus | |
| Molds | Stachybotrys | idiopathic pulmonary hemorrhage (infants) |
| Molds | Trichophyton | Tiñea pedis, corporis, cruris, onychomycosis and occasionally |
| | interdigitale | Tinea capitis |
| Molds | Trichophyton | Tinea of the groin, glabrous skin, feet, hands, and the nails. Tinea |
| | rubrum | cruris, tinea corporis, tinea pedis, tinea manuum, and |
| | | onychomycosis |
| Dimorphic | Histoplasma | Pneumonia, multi-organ failure |
| Fungi | capsulatum | |
| Dimorphic | Pneumocystis | Pneumonia (mostly in immune supressed patients) |
| Fungi | jirovecii | |
| Dimorphic | Paracoccidioides | Disseminated systemic infection |
| Fungi | brasiliensis | |
| Dimorphic | Penicillium | Disseminated infection with prominent skin lesions |
| Fungi | marneffei | (immunocompromised patients) |
| Dimorphic | Blastomyces | Skin infections, rearely disseminated |
| Fungi | | |
| Dimorphic | Coccidioides | Meningitis, disseminated disease, lung nodule, chronic cavitary |
| Fungi | | pulmonary coccidioidmycosis |

According to some embodiments, the peptide is a parasitic antigen. According to some embodiments, the disclosure provides a ceDNA as described herein comprising a nucleic acid sequence that encodes one or more fungal antigens.

Exemplary parasitic infections are shown in Table 7 below.

TABLE 7

| Parasitic | | |
|---|---|---|
| Group | Pathogen Species | Disease / Pathology Example |
| Amoeba / | Acanthamoeba | Acanthamoeba Infection |
| Protozoa | | |
| Amoeba / | Acanthamoeba | Acanthamoeba Keratitis Infection |
| Protozoa | | |
| Amoeba / | Trypanosoma brucei | African Sleeping Sickness (African trypanosomiasis) |
| Protozoa | | |
| Amoeba / | Entamoeba histolytica | Amebiasis (Entamoeba histolytica Infection) |
| Protozoa | | |
| Amoeba / | Trypanosoma cruzi | American Trypanosomiasis (Chagas Disease) |
| Protozoa | | |
| Amoeba / | Balantidium coli | Balantidiasis (Balantidium Infection) |
| Protozoa | | |
| Amoeba / | Balamuthia mandrillaris | Balamuthia (Granulomatous Amebic Encephalitis (GAE)) |
| Protozoa | | |
| Amoeba / | Cryptosporidium | Cryptosporidiosis (Cryptosporidium Infection) |
| Protozoa | | |
| Amoeba / | Cyclospora | Cyclosporiasis (Cyclospora Infection) |
| Protozoa | | |
| Amoeba / | Taenia solium | Cysticercosis (Neurocysticercosis) |
| Protozoa | | |
| Amoeba / | Cystoisospora belli | Cystoisospora Infection (Cystoisosporiasis) formerly Isospora |
| Protozoa | | Infection |
| Amoeba / | Dientamoeba fragilis | Dientamoeba fragilis Infection |
| Protozoa | | |
| Amoeba / | Entamoeba histolytica | Entamoeba histolytica Infection (Amebiasis) |
| Protozoa | | |
| Amoeba / | Giardia | Giardiasis (Giardia Infection) |
| Protozoa | intestinalis, Giardia | |
| | lamblia, or Giardia | |
| | duodenalis | |
| Amoeba / | Leishmania | Kala-azar (Leishmaniasis, Leishmania Infection) |
| Protozoa | | |
| Amoeba / | Acanthamoeba | Keratitis (Acanthamoeba Infection) |
| Protozoa | | |
| Amoeba / | Multiple species. | Malaria (Plasmodium Infection) |
| Protozoa | Examples: P. vivax and | |
| | P. ovale | |
| Amoeba / | Naegleria fowleri | Naegleria Infection |
| Protozoa | “brain-eating amoeba” | |
| Amoeba / | Sappinia ameoba | Sappinia (amebic encephalitis) |
| Protozoa | | |
| Amoeba / | Sarcocystis | Sarcocystosis (Sarcocystosis Infection) |
| Protozoa | | |
| Amoeba / | Toxoplasma gondii | Toxoplasmosis (Toxoplasma Infection) - protazoa |
| Protozoa | | |
| Amoeba / | Trichomonas vaginalis | Trichomoniasis (Trichomonas Infection) - protozoa |
| Protozoa | | |
| Amoeba / | Trypanosoma brucei | Trypanosomiasis, African (African Sleeping Sickness, Sleeping |
| Protozoa | | Sickness) - protozoa |
| Amoeba / | Babesia (various | Babesiosis (Babesia Infection) - similar lifecycle to malaria, |
| Protozoa | species) | taken up by RBCs |
| Arthropod | Cimex lectulariusand C. | Bed Bugs |
| | hemipterus | |
| Arthropod | Pediculus humanus | Body Lice Infestation (Pediculosis) |
| | humanus | |
| Arthropod | Phthirus pubis | Crabs (Pubic Lice) |
| Arthropod | Pediculus humanus | Head Lice Infestation (Pediculosis) |
| | capitis | |
| Arthropod | Sarcoptes | Mite Infestation (Scabies) |
| | scabiei var. hominis | |
| Arthropod | infection of a fly larva. | Myiasis |
| | Examples: Dermatobia | |
| | hominis | |
| Other | group of obligate | Microsporidiosis (Microsporidia Infection) |
| | intracellular parasitic | |
| | fungi. Examples: M. | |
| | africanum, Nosema | |
| | ocularum | |
| Other | Pneumocystis jirovecii | Pneumocystis jirovecii Pneumonia |
| | (considered a parasitic | |
| | fungi) | |
| Protist | Blastocystis hominis | Blastocystis hominis Infection |
| Worm | Echinococcus | Alveolar Echinococcosis (Echinococcosis, Hydatid Disease) |
| | granulosus | |
| Worm | Hookworm (various | Ancylostomiasis (Hookworm) |
| | species) | |
| Worm | Angiostrongylus | Angiostrongyliasis (Angiostrongylus Infection) |
| | (various species) | |
| | nematode | |
| Worm | anisakid nematodes | Anisakiasis (Anisakis Infection, Pseudoterranova Infection) |
| Worm | Ascaris lumbricoides | Ascariasis (Ascaris Infection, Intestinal Roundworms) |
| Worm | B. procyonis | Baylisascariasis (Baylisascaris Infection, Raccoon Roundworm) |
| Worm | S. mansoni | Bilharzia (Schistosomiasis) |
| Worm | Capillaria | Capillariasis (Capillaria Infection) |
| | hepatica and Capillaria | |
| | philippinensis | |
| Worm | Various species, | Cercarial Dermatitis (Swimmer's Itch) |
| | example: | |
| | Austrobilharzia | |
| | variglandis | |
| Worm | Clonorchis liver fluke | Clonorchiasis (Clonorchis Infection) |
| Worm | Diphyllobothrium | Diphyllobothriasis (Diphyllobothrium Infection) |
| | latum (tapeworm) | |
| Worm | Dipylidiu tapeworm | Dipylidium caninum Infection (dog or cat tapeworm infection) |
| Worm | Dirofilaria roundworms | Dirofilariasis (Dirofilaria Infection) |
| Worm | Guinea worm | Dracunculiasis (Guinea Worm Disease) |
| Worm | Echinococcus | Echinococcosis (Cystic, Alveolar Hydatid Disease) |
| | granulosus & | |
| | Echinococcus | |
| | multilocularis | |
| Worm | Wuchereria | Elephantiasis (Filariasis, Lymphatic Filariasis) |
| | bancrofti, Brugia malayi | |
| | and Brugia timori | |
| Worm | Enterobius vermicularis | Enterobiasis (Pinworm Infection) |
| Worm | Fasciola hepatica | Fascioliasis (Fasciola(liver fluke) Infection) |
| Worm | Fasciolopsis buski | Fasciolopsiasis (Fasciolopsis Infection) |
| Worm | Gnathostoma: several | Gnathostomiasis (Gnathostoma Infection) |
| | species of parasitic | |
| | nematodes | |
| Worm | trematode Heterophyes | Heterophyiasis (Heterophyes Infection) |
| | heterophyes | |
| Worm | Multiple species. | Hookworm Infection, Human |
| | Example: L: Filariform | |
| Worm | Multiple species. | Hookworm Infection, Zoonotic (Ancylostomiasis, Cutaneous |
| | Example: Ancylostoma | Larva Migrans [CLM]) |
| | brazilense, A. caninum, | |
| | A. ceylanicum | |
| Worm | H. nana (dwarf | Hymenolepiasis (Hymenolepis Infection) |
| | tapeworm) | |
| Worm | Various species, | Intestinal Roundworms (Ascariasis, Ascaris Infection) |
| | example: A. | |
| | lumbricoides | |
| Worm | Loa loa: parasitic | Loiasis (Loa loa Infection) |
| | worm | |
| Worm | Taenia solium | Neurocysticercosis (Cysticercosis) |
| Worm | Toxocara canis and | Ocular Larva Migrans (Toxocariasis, Toxocara Infection, |
| | Toxocara cati | Visceral Larva Migrans) |
| Worm | Onchocerca volvulus | Onchocerciasis (River Blindness) |
| Worm | Opisthorchis (liver | Opisthorchiasis (Opisthorchis Infection) |
| | fluke). Example: O. | |
| | felineus | |
| Worm | Paragonimus(lung | Paragonimiasis (Paragonimus Infection) |
| | fluke) | |
| Worm | anisakid nematodes | Pseudoterranova Infection (Anisakiasis, Anisakis Infection) |
| Worm | Strongyloides | Strongyloidiasis (Strongyloides Infection) |
| | nematodes. Example: S. | |
| | stercoralis | |
| Worm | Taenia saginata | Taeniasis (Taenia Infection, Tapeworm Infection) |
| Worm | Trichinella | Trichinellosis (Trichinosis) |
| Worm | T. trichiura | Trichuriasis (Whipworm Infection, Trichuris Infection) |

Other diseases and disorders are contemplated for treatment by the ceDNA vectors of the present disclosure. Examples include, but are not limited to, cardiovascular diseases and immune diseases.

It is well within the abilities of one of skill in the art to take a known and/or publically available protein sequence of e.g., an antigen, and reverse engineer a cDNA sequence to encode such a protein.

### IV. ceDNA Vector For Use In Production of Antigens

Embodiments of the disclosure are based on methods and compositions comprising a priming vaccine comprising a deoxyribonucleic acid (DNA) DNA, wherein the DNA is close ended linear duplexed (ceDNA) vectors that can express peptides. As described herein, peptides (e.g., antigens) may be selected from a variety of pathogens, including, e.g., bacterial, viral, fungal and parasitic infectious agents, or cancer or cancer-associated antigens, or the like. Still other targets may include an autoimmune condition such as rheumatoid arthritis (RA) or multiple sclerosis (MS).

According to some embodiments, the transgene is a nucleic acid sequence encoding an antigen. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In general, a ceDNA vector for expression of peptides (e.g., antigens) as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

Encompassed herein are methods and compositions comprising the ceDNA vector for production of peptides (e.g., antigens) which may further include a delivery system, such as but not limited to, a liposome nanoparticle delivery system. Non-limiting exemplary liposome nanoparticle systems encompassed for use are disclosed herein. According to some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, which is incorporated herein.

The ceDNA vectors as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes.

This permits the insertion of control elements, e.g., regulatory switches as disclosed herein, large transgenes, multiple transgenes etc.

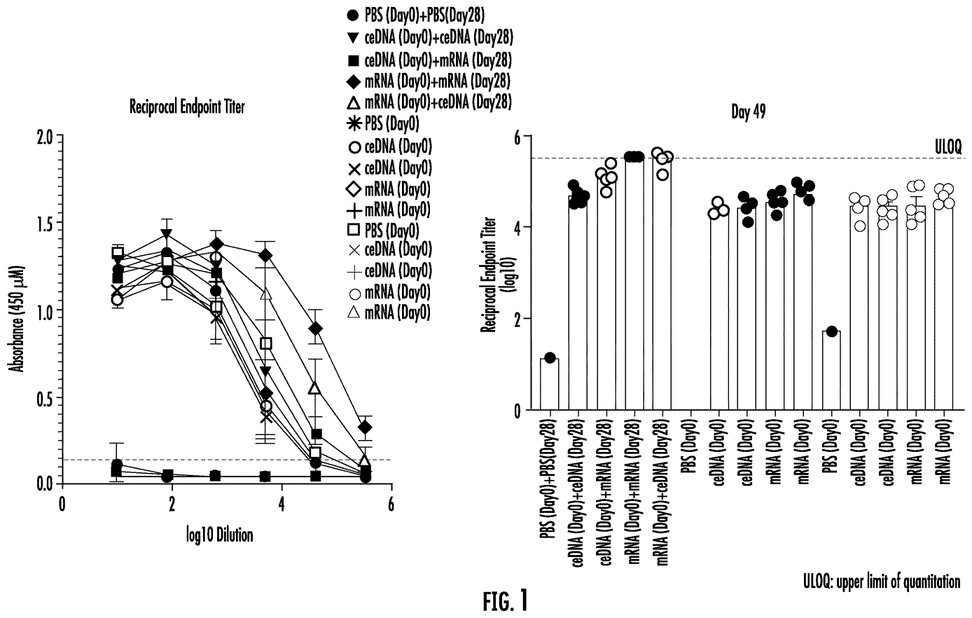

FIGS. 1A-1E of International Publication No. WO/2019/051255, incorporated by reference in its entirety herein, show schematics of non-limiting, exemplary ceDNA vectors for expression of peptides (e.g., antigens) or the corresponding sequence of ceDNA plasmids. ceDNA vectors for expression of peptides (e.g., antigens) are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter (transgene), a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. According to some embodiments the ITR can act as the promoter for the transgene. According to some embodiments, the ceDNA vector comprises additional components to regulate expression of the transgene, for example, a regulatory switch, for controlling and regulating the expression of the peptides (e.g., antigens) and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. According to some embodiments, the expression cassette can comprise a transgene in the range of 500 to 50,000 nucleotides in length. According to some embodiments, the expression cassette can comprise a transgene in the range of 500 to 75,000 nucleotides in length. According to some embodiments, the expression cassette can comprise a transgene which is in the range of 500 to 10,000 nucleotides in length. According to some embodiments, the expression cassette can comprise a transgene which is in the range of 1000 to 10,000 nucleotides in length. According to some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene expression. According to some embodiments, the ceDNA vector is devoid of prokaryote-specific methylation.

Sequences provided in the expression cassette, expression construct of a ceDNA vector for expression of peptides (e.g., antigens) described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Hemdon, Va. 20171) or another publicly available database. According to some embodiments, the nucleic acid is optimized for human expression.

A transgene expressed by the ceDNA vector for expression of peptides (e.g., antigens) as disclosed herein encodes antigens. There are many structural features of ceDNA vectors that differ from plasmid-based expression vectors. ceDNA vectors may possess one or more of the following features: the lack of original (i.e., not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region. Another important feature distinguishing ceDNA vectors from plasmid expression vectors is that ceDNA vectors are single-strand linear DNA having closed ends, while plasmids are always double-strand DNA.

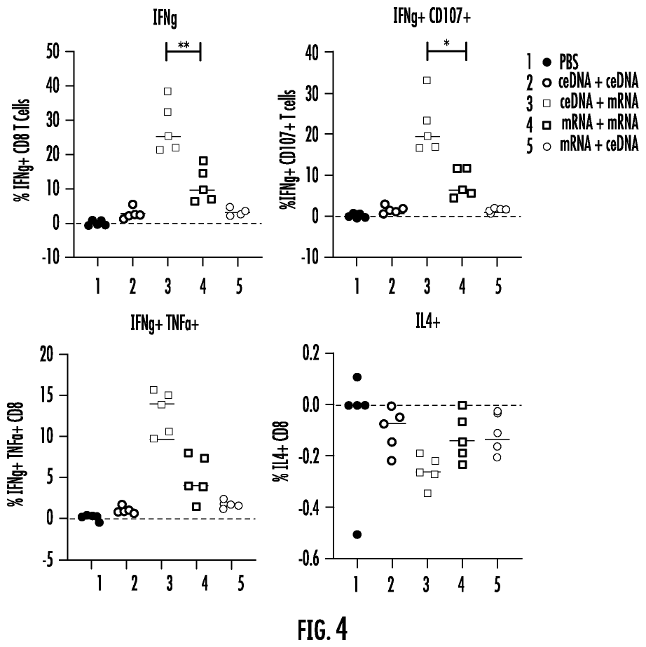

ceDNA vectors for expression of peptides (e.g., antigens) produced by the methods provided herein preferably have a linear and continuous structure rather than a non-continuous structure, as determined by restriction enzyme digestion assay (see, e.g., FIG. 4D of International Publication No. WO/2019/051255, incorporated by reference in its entirety herein). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. According to some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several differences of using a ceDNA vector for expression of peptides (e.g., antigens) from plasmid-based expression vectors, such differences include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus.

### Inverted Terminal Repeats (ITRs)

As disclosed herein, ceDNA vectors for expression of peptides (e.g., antigens) contain a transgene or nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

According to some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. According to some embodiments, the AAV can infect warm-blooded animals, e.g., avian (AAAV), bovine (BAAV), canine, equine, and ovine adeno-associated viruses. According to some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). According to some embodiments, the 5′ WT-ITR can be from one serotype and the 3′ WT-ITR from a different serotype, as discussed herein.

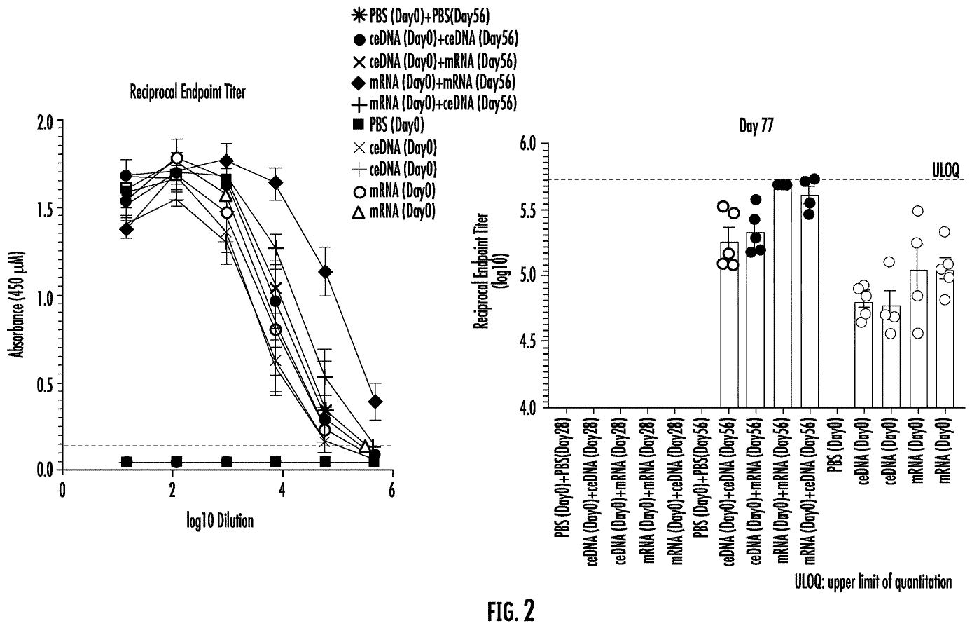

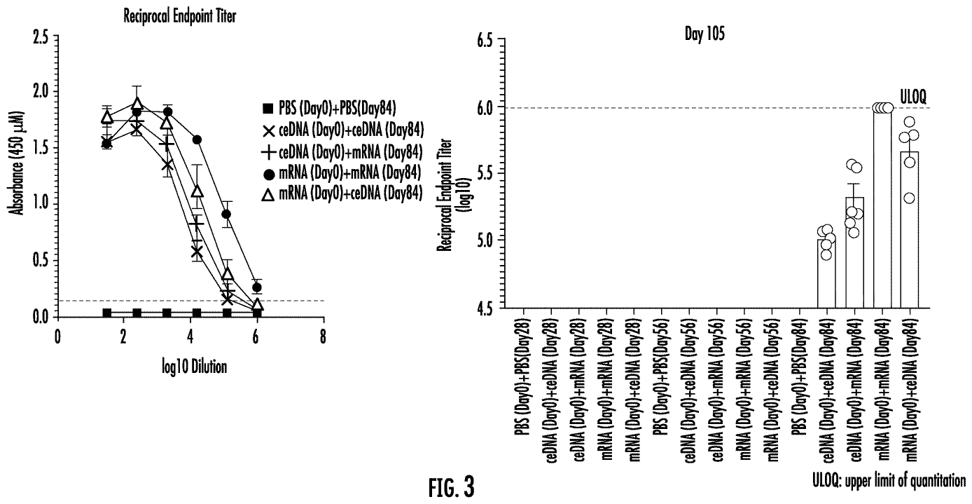

An ordinarily skilled artisan is aware that ITR sequences have a common structure of a double-stranded Holliday junction, which typically is a T-shaped or Y-shaped hairpin structure (see e.g., FIG. 2A and FIG. 3A of International Publication No. WO/2019/051255, incorporated by reference in its entirety herein), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### Symmetrical ITR Pairs

According to some embodiments, a ceDNA vector for expression of peptides (e.g., antigens) as described herein comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

### (i) Wildtype ITRs

According to some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, according to some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a transgene or nucleic acid sequence positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. According to some embodiments, a wild-type ITR sequence (e.g., AAV WT-ITR) comprises a functional Rep binding site (RBS; e.g., 5′-GCGCGCTCGCTCGCTC-3′ for AAV2, SEQ ID NO: __) and a functional terminal resolution site (TRS; e.g., 5′-AGTT-3′, SEQ ID NO: __).

According to some aspect, ceDNA vectors for expression of peptides (e.g., antigens) are obtainable from a vector polynucleotide that encodes a nucleic acid operatively positioned between two WT inverted terminal repeat sequences (WT-ITRs) (e.g., AAV WT-ITRs). That is, both ITRs have a wild type sequence, but do not necessarily have to be WT-ITRs from the same AAV serotype. That is, according to some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. According to some embodiments, the 5′ WT-ITR is from one AAV serotype, and the 3′ WT-ITR is from the same or a different AAV serotype. According to some embodiments, the 5′ WT-ITR and the 3′WT-ITR are mirror images of each other, that is they are symmetrical. According to some embodiments, the 5′ WT-ITR and the 3′ WT-ITR are from the same AAV serotype.

WT ITRs are well known. According to some embodiment the two ITRs are from the same AAV2 serotype. In certain embodiments one can use WT from other serotypes. There are a number of serotypes that are homologous, e.g., AAV2, AAV4, AAV6, AAV8. According to some embodiments, closely homologous ITRs (e.g., ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA, e.g., the expression of the encoded antigens, or immunogenic peptides.

According to some embodiments, one aspect of the technology described herein relates to a ceDNA vector for expression of peptides (e.g., antigens) wherein the ceDNA vector comprises at least one nucleic acid sequence encoding, e.g., a HC and/or a LC, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). According to some embodiments, the symmetric WT-ITRs comprises a functional terminal resolution site and a Rep binding site. According to some embodiments, the nucleic acid sequence encodes a transgene, and wherein the vector is not in a viral capsid.

According to some embodiments, the WT-ITRs are the same but the reverse complement of each other. For example, the sequence AACG in the 5′ ITR may be CGTT (i.e., the reverse complement) in the 3′ ITR at the corresponding site. According to some example, the 5′ WT-ITR sense strand comprises the sequence of ATCGATCG and the corresponding 3′ WT-ITR sense strand comprises CGATCGAT (i.e., the reverse complement of ATCGATCG). According to some embodiments, the WT-ITRs ceDNA further comprises a terminal resolution site and a replication protein binding site (RPS) (sometimes referred to as a replicative protein binding site), e.g., a Rep binding site.

Exemplary WT-ITR sequences for use in the ceDNA vectors for expression of peptides (e.g., antigens) comprising WT-ITRs are shown in Table 8 herein, which shows pairs of WT-ITRs (5′ WT-ITR and the 3′ WT-ITR).

As an exemplary example, the present disclosure provides a ceDNA vector for expression of peptides (e.g., antigens) comprising a promoter operably linked to a transgene (e.g., nucleic acid sequence), with or without the regulatory switch, where the ceDNA is devoid of capsid proteins and is: (a) produced from a ceDNA-plasmid (e.g., see FIGS. 1F-1G of International Publication No. WO/2019/051255, incorporated by reference in its entirety herein) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1.

According to some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g., AAV1, 3, 4, 5, 6, 7, 8, 9, 10, 11, and 12. According to some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. According to some embodiments, such a combination of WT ITRs is the combination of WT-ITRs from AAV2 and AAV6. According to some embodiments, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization.

According to some embodiments, a WT-ITR pair are substantially symmetrical as they have symmetrical three-dimensional spatial organization, e.g., have the same 3D organization of the A, C-C′. B-B′ and D arms. According to some embodiments, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs). According to some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: __) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

According to some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleic acid sequence of the ITR, a spacing between two or more elements, or a combination of any of the above. According to some embodiments, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 8 indicates exemplary combinations of WT-ITRs.

Table 8: Exemplary combinations of WT-ITRs from the same serotype or different serotypes, or different parvoviruses. The order shown is not indicative of the ITR position, for example, “AAV 1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 Parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 8
Exemplary combinations of WT-ITRs

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 9 shows the sequences of exemplary WT-ITRs from some different AAV serotypes.

TABLE 9
Exemplary WT-ITRs

| AAV | | |
|---|---|---|
| serotype | 5′ WT-ITR (LEFT) | 3′ WT-ITR (RIGHT) |
| AAVI | 5′-TTGCCCACTCCCTCTCTGCGCGCTCGCT | 5′-TTACCCTAGTGATGGAGTTGCCCACTCCC |
| | CGCTCGGTGGGGCCTGCGGACCAAAGG | TCTCTGCGCGCGTCGCTCGCTCGGTGGG |
| | TCCGCAGACGGCAGAGGTCTCCTCTGCC | GCCGGCAGAGGAGACCTCTGCCGTCTGC |
| | GGCCCCACCGAGCGAGCGACGCGCGCA | GGACCTTTGGTCCGCAGGCCCCACCGAG |
| | GAGAGGGAGTGGGCAACTCCATCACTA | CGAGCGAGCGCGCAGAGAGGGAGTGGG |
| | GGGTAA-3′ | CAA-3′ |
| | (SEQ ID NO: 5) | (SEQ ID NO: 10) |
| AAV2 | CCTGCAGGCAGCTGCGCGCTCGCTCGCT | AGGAACCCCTAGTGATGGAGTTGGCCAC |
| | CACTGAGGCCGCCCGGGCAAAGCCCGG | TCCCTCTCTGCGCGCTCGCTCGCTCACTG |
| | GCGTCGGGCGACCTTTGGTCGCCCGGCC | AGGCCGGGCGACCAAAGGTCGCCCGAC |
| | TCAGTGAGCGAGCGAGCGCGCAGAGAG | GCCCGGGCTTTGCCCGGGCGGCCTCAGT |
| | GGAGTGGCCAACTCCATCACTAGGGGTTCCT | GAGCGAGCGAGCGCGCAGCTGCCTGCAGG |
| | (SEQ ID NO: 2) | (SEQ ID NO: 1) |
| AAV3 | 5′-TTGGCCACTCCCTCTATGCGCACTCGCT | 5′-ATACCTCTAGTGATGGAGTTGGCCACTC |
| | CGCTCGGTGGGGCCTGGCGACCAAAGG | CCTCTATGCGCACTCGCTCGCTCGGTGG |
| | TCGCCAGACGGACGTGGGTTTCCACGTC | GGCCGGACGTGGAAACCCACGTCCGTCT |
| | CGGCCCCACCGAGCGAGCGAGTGCGCA | GGCGACCTTTGGTCGCCAGGCCCCACCG |
| | TAGAGGGAGTGGCCAACTCCATCACTA | AGCGAGCGAGTGCGCATAGAGGGAGTG |
| | GAGGTAT-3′ | GCCAA-3′ |
| | (SEQ ID NO: 6) | (SEQ ID NO: 11) |
| AAV4 | 5′-TTGGCCACTCCCTCTATGCGCGCTCGCT | 5′-AGTTGGCCACATTAGCTATGCGCGCTCG |
| | CACTCACTCGGCCCTGGAGACCAAAGG | CTCACTCACTCGGCCCTGGAGACCAAAG |
| | TCTCCAGACTGCCGGCCTCTGGCCGGCA | GTCTCCAGACTGCCGGCCTCTGGCCGGC |
| | GGGCCGAGTGAGTGAGCGAGCGCGCAT | AGGGCCGAGTGAGTGAGCGAGCGCGCA |
| | AGAGGGAGTGGCCAACT-3′ | TAGAGGGAGTGGCCAA-3′ |
| | (SEQ ID NO: 7) | (SEQ ID NO: 12) |
| AAV5 | 5′-TCCCCCCTGTCGCGTTCGCTCGCTCGCT | 5′-CTTACAAAACCCCCTTGCTTGAGAGTGT |
| | GGCTCGTTTGGGGGGGCGACGGCCAGA | GGCACTCTCCCCCCTGTCGCGTTCGCTCG |
| | GGGCCGTCGTCTGGCAGCTCTTTGAGCT | CTCGCTGGCTCGTTTGGGGGGGTGGCAG |
| | GCCACCCCCCCAAACGAGCCAGCGAGC | CTCAAAGAGCTGCCAGACGACGGCCCTC |
| | GAGCGAACGCGACAGGGGGGAGAGTGC | TGGCCGTCGCCCCCCCAAACGAGCCAGC |
| | CACACTCTCAAGCAAGGGGGTTTTGTAAG-3′ | GAGCGAGCGAACGCGACAGGGGGGA-3′ |
| | (SEQ ID NO: 8) | (SEQ ID NO: 13) |
| AAV6 | 5′-TTGCCCACTCCCTCTAATGCGCGCTCGC | 5′-ATACCCCTAGTGATGGAGTTGCCCACTC |
| | TCGCTCGGTGGGGCCTGCGGACCAAAG | CCTCTATGCGCGCTCGCTCGCTCGGTGG |
| | GTCCGCAGACGGCAGAGGTCTCCTCTGC | GGCCGGCAGAGGAGACCTCTGCCGTCTG |
| | CGGCCCCACCGAGCGAGCGAGCGCGCA | CGGACCTTTGGTCCGCAGGCCCCACCGA |
| | TAGAGGGAGTGGGCAACTCCATCACTA | GCGAGCGAGCGCGCATTAGAGGGAGTG |
| | GGGGTAT-3′ | GGCAA |
| | (SEQ ID NO: 9) | (SEQ ID NO: 14) |

According to some embodiments, the nucleic acid sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present disclosure, the ceDNA vector for expression of peptides (e.g., antigens) does not have a WT-ITR consisting of the nucleic acid sequence selected from any of: SEQ ID NOs: 1, 2, 5-14. In alternative embodiments of the present disclosure, if a ceDNA vector has a WT-ITR comprising the nucleic acid sequence selected from any of: SEQ ID NOs: 1, 2, 5-14, then the flanking ITR is also WT and the ceDNA vector comprises a regulatory switch, e.g., as disclosed herein and in International application PCT/US18/49996 (e.g., see Table 11 of PCT/US18/49996, incorporated by reference in its entirety herein). According to some embodiments, the ceDNA vector for expression of peptides (e.g., antigens) comprises a regulatory switch as disclosed herein and a WT-ITR selected having the nucleic acid sequence selected from any of the group consisting of: SEQ ID NO: 1, 2, 5-14.

The ceDNA vector for expression of peptides (e.g., antigens) as described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. According to some embodiments, the ceDNA vector for expression of peptides (e.g., antigens) contains one or more functional WT-ITR polynucleotide sequences that comprise a Rep-binding site (RBS; 5′-GCGCGCTCGCTCGCTC-3′ (SEQ ID NO: __) for AAV2) and a terminal resolution site (TRS; 5′-AGTT (SEQ ID NO: __)). According to some embodiments, at least one WT-ITR is functional. In alternative embodiments, where a ceDNA vector for expression of peptides (e.g., antigens) comprises two WT-ITRs that are substantially symmetrical to each other, at least one WT-ITR is functional and at least one WT-ITR is non-functional.

Modified ITRs (Mod-ITRs) in General for ceDNA Vectors Comprising Asymmetric ITR Pairs or Symmetric ITR Pairs

As discussed herein, a ceDNA vector for expression of peptides (e.g., antigens) can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′and B-B′ arms).

According to some embodiments, a modified ITR is an ITRs that is modified by deletion, insertion, and/or substitution as compared to a wild-type ITR sequence (e.g., AAV ITR). According to some embodiments, at least one of the ITRs in the ceDNA vector comprises a functional Rep binding site (RBS; e.g., 5′-GCGCGCTCGCTCGCTC-3′ for AAV2) and a functional terminal resolution site (TRS; e.g., 5′-AGTT-3′) According to some embodiments, at least one of the ITRs is a non-functional ITR. According to some embodiments, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

According to some embodiments, a mod-ITR may be synthetic. According to some embodiments, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. According to some aspects, a synthetic ITR may interact preferentially with a wild type Rep or a Rep of a specific serotype, or According to some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The disclosure further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, according to some embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV 11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. According to some embodiments, the modified ITR is based on an AAV2 ITR.

More specifically, the ability of a structural element to functionally interact with a particular large Rep protein can be altered by modifying the structural element. For example, the nucleic acid sequence of the structural element can be modified as compared to the wild-type sequence of the ITR. According to some embodiments, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV 11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 10 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. According to some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. According to some embodiments, a truncated C-C′ arm and/or a truncated B-B′ arm has three sequential T nucleotides (i.e., TTT) in the terminal loop.

TABLE 10
Exemplary combinations of modifications of at least one nucleotide
(e.g., a deletion, insertion and/or substitution) to different
B-B′ and C-C′ regions or arms of ITRs (X indicates
a nucleotide modification, e.g., addition, deletion or substitution
of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

According to some embodiments, mod-ITR for use in a ceDNA vector for expression of Peptides (e.g., antigens) comprises an asymmetric ITR pair, or a symmetric mod-ITR pair as disclosed herein, can comprise any one of the combinations of modifications shown in Table 10, and also a modification of at least one nucleotide in any one or more of the regions selected from: between A′ and C, between C and C′, between C′ and B, between B and B′ and between B′ and A. According to some embodiments, any modification of at least one nucleotide (e.g., a deletion, insertion and/or substitution) in the C or C′ or B or B′ regions, still preserves the terminal loop of the stem-loop. According to some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop. According to some embodiments, a modified ITR for use herein can comprise any one of the combinations of modifications shown in Table 10, and also a modification of at least one nucleotide (e.g., a deletion, insertion and/or substitution) in any one or more of the regions selected from: A′, A and/or D. For example, according to some embodiments, a modified ITR for use herein can comprise any one of the combinations of modifications shown in Table 10, and also a modification of at least one nucleotide (e.g., a deletion, insertion and/or substitution) in the A region. According to some embodiments, a modified ITR for use herein can comprise any one of the combinations of modifications shown in Table 10, and also a modification of at least one nucleotide (e.g., a deletion, insertion and/or substitution) in the A′ region. According to some embodiments, a modified ITR for use herein can comprise any one of the combinations of modifications shown in Table 10, and also a modification of at least one nucleotide (e.g., a deletion, insertion and/or substitution) in the A and/or A′ region. According to some embodiments, a modified ITR for use herein can comprise any one of the combinations of modifications shown in Table 10, and also a modification of at least one nucleotide (e.g., a deletion, insertion and/or substitution) in the D region.

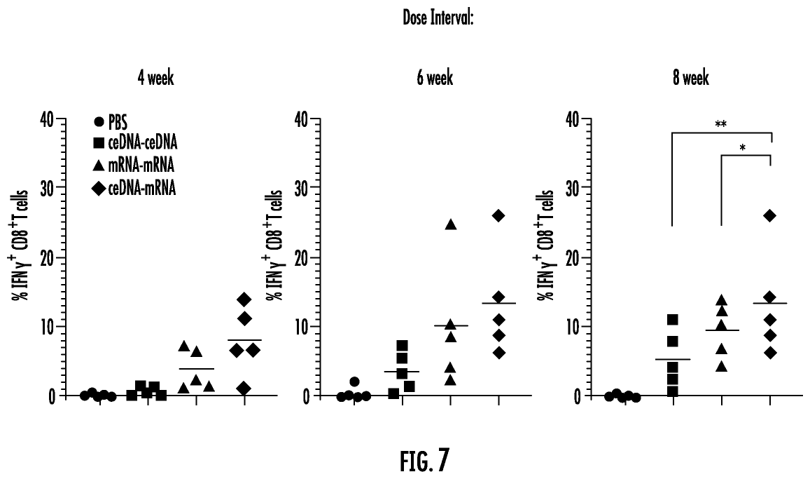

According to some embodiments, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. According to some embodiments, the specific modifications to the ITRs are exemplified herein (e.g., SEQ ID NOS: 3, 4, 15-47, 101-116 or 165-187, or shown in FIGS. 7A-7B of International Patent Application No. PCT/US2018/064242, filed on Dec. 6, 2018 (e.g., SEQ ID Nos 97-98, 101-103, 105-108, 111-112, 117-134, 545-54 in PCT/US2018/064242). According to some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International Patent Application No. PCT/US18/49996, which is incorporated herein in its entirety by reference.

According to some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of PCT/US2018/064242, filed Dec. 6, 2018). According to some embodiments, a modified ITR can comprise the removal of 1, 2, 3, 4, 5, 6, 7, 8, 9 or more base pairs from the B-B′ arm. According to some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of International Patent Application No. PCT/US2018/064242, filed Dec. 6, 2018). According to some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. According to some embodiments, the modified ITR also comprises at least one base pair deletion from each of the B region and B′ regions, such that the B-B′ arm is also truncated relative to WT ITR.

According to some embodiments, a modified ITR can have between 1 and 50 (e.g., 1, 2, 3, 4, 5,6,7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. According to some embodiments, a modified ITR can have between 1 and 30 nucleotide deletions relative to a full-length WT ITR sequence. According to some embodiments, a modified ITR has between 2 and 20 nucleotide deletions relative to a full-length wild-type ITR sequence.

According to some embodiments, a modified ITR does not contain any nucleotide deletions in the RBE-containing portion of the A or A′ regions, so as not to interfere with DNA replication (e.g., binding to an RBE by Rep protein, or nicking at a terminal resolution site). According to some embodiments, a modified ITR encompassed for use herein has one or more deletions in the B, B′, C, and/or C region as described herein.

According to some embodiments, a ceDNA vector for expression of peptides (e.g., antigens) comprising a symmetric ITR pair or asymmetric ITR pair comprises a regulatory switch as disclosed herein and at least one modified ITR selected having the nucleotide sequence selected from any of the group consisting of: SEQ ID NO: 3, 4, 15-47, 101-116 or 165-187.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. According to some embodiments, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. According to some example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector for expression of peptides (e.g., antigens) as described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector for expression of antigens, or immunogenic peptides. According to some embodiments, the ceDNA vector for expression of peptides (e.g., antigens) contains one or more functional ITR polynucleotide sequences that comprise a Rep-binding site (RBS; 5′-GCGCGCTCGCTCGCTC-3′ for AAV2) and a terminal resolution site (TRS; 5′-AGTT). According to some embodiments, at least one ITR (wt or modified ITR) is functional. In alternative embodiments, where a ceDNA vector for expression of peptides (e.g., antigens) comprises two modified ITRs that are different or asymmetrical to each other, at least one modified ITR is functional and at least one modified ITR is non-functional.

According to some embodiments, the modified ITR (e.g., the left or right ITR) of a ceDNA vector for expression of peptides (e.g., antigens) as described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318 herein); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of International Patent Application No. PCT/US18/49996, which is incorporated herein in its entirety by reference.

According to some embodiments, the modified ITR for use in a ceDNA vector for expression of peptides (e.g., antigens) comprising an asymmetric ITR pair, or symmetric mod-ITR pair is selected from any or a combination of those shown in Tables 2, 3, 4, 5, 6, 7, 8, 9 and 10A-10B of International Patent Application No. PCT/US18/49996 which is incorporated herein in its entirety by reference.

Additional exemplary modified ITRs for use in a ceDNA vector for expression of peptides (e.g., antigens) comprising an asymmetric ITR pair, or symmetric mod-ITR pair in each of the above classes are provided in Tables 11A and 11B. The predicted secondary structure of the Right modified ITRs in Table 11A are shown in FIG. 7A of International Patent Application No. PCT/US2018/064242, filed Dec. 6, 2018, and the predicted secondary structure of the Left modified ITRs in Table 11B are shown in FIG. 7B of International Patent Application No. PCT/US2018/064242, filed Dec. 6, 2018, which is incorporated herein in its entirety by reference.

Table 11A and Table 11B list the SEQ ID NOs of exemplary right and left modified ITRs.

TABLE 11A
Exemplary modified right ITRs. These exemplary modified right ITRs
can comprise the RBE of GCGCGCTCGCTCGCTC-3′ (spacer of ACTGAGGC), the
spacer complement GCCTCAGT and RBE′ (i.e., complement to RBE)
of GAGCGAGCGAGCGCGC.

| ITR | | SEQ ID |
|---|---|---|
| Construct | Sequence | NO: |
| ITR-18 | AGGAACCCCTAGTGATGGAGTTGGCCACTCCCTCTCTGCGCGCTCG | 15 |
| Right | CTCGCTCACTGAGGCGCACGCCCGGGTTTCCCGGGCGGCCTCAGTG | |
| | AGCGAGCGAGCGCGCAGCTGCCTGCAGG | |
| ITR-19 | AGGAACCCCTAGTGATGGAGTTGGCCACTCCCTCTCTGCGCGCTCG | 16 |
| Right | CTCGCTCACTGAGGCCGACGCCCGGGCTTTGCCCGGGCGGCCTCA | |
| | GTGAGCGAGCGAGCGCGCAGCTGCCTGCAGG | |
| ITR-20 | AGGAACCCCTAGTGATGGAGTTGGCCACTCCCTCTCTGCGCGCTCG | 17 |
| Right | CTCGCTCACTGAGGCCGGGCGACCAAAGGTCGCCCGACGCCCGGG | |
| | CGCCTCAGTGAGCGAGCGAGCGCGCAGCTGCCTGCAGG | |
| ITR-21 | AGGAACCCCTAGTGATGGAGTTGGCCACTCCCTCTCTGCGCGCTCG | 18 |
| Right | CTCGCTCACTGAGGCTTTGCCTCAGTGAGCGAGCGAGCGCGCAGC | |
| | TGCCTGCAGG | |
| ITR-22 | AGGAACCCCTAGTGATGGAGTTGGCCACTCCCTCTCTGCGCGCTCG | 19 |
| Right | CTCGCTCACTGAGGCCGGGCGACAAAGTCGCCCGACGCCCGGGCT | |
| | TTGCCCGGGCGGCCTCAGTGAGCGAGCGAGCGCGCAGCTGCCTGC | |
| | AGG | |
| ITR-23 | AGGAACCCCTAGTGATGGAGTTGGCCACTCCCTCTCTGCGCGCTCG | 20 |
| Right | CTCGCTCACTGAGGCCGGGCGAAAATCGCCCGACGCCCGGGCTTT | |
| | GCCCGGGCGGCCTCAGTGAGCGAGCGAGCGCGCAGCTGCCTGCAGG | |
| ITR-24 | AGGAACCCCTAGTGATGGAGTTGGCCACTCCCTCTCTGCGCGCTCG | 21 |
| Right | CTCGCTCACTGAGGCCGGGCGAAACGCCCGACGCCCGGGCTTTGC | |
| | CCGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCG | 22 |
| Right | CTCGCTCACTGAGGCCGGGCAAAGCCCGACGCCCGGGCTTTGCCC | |
| | GGGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCG | 23 |
| Right | CTCGCTCACTGAGGCCGGGCGACCAAAGGTCGCCCGACGCCCGGG | |
| | TTTCCCGGGCGGCCTCAGTGAGCGAGCGAGCGCGCAGCTGCCTGC | |
| | AGG | |
| ITR-27 | AGGAACCCCTAGTGATGGAGTTGGCCACTCCCTCTCTGCGCGCTCG | 24 |
| Right | CTCGCTCACTGAGGCCGGGCGACCAAAGGTCGCCCGACGCCCGGT | |
| | TTCCGGGCGGCCTCAGTGAGCGAGCGAGCGCGCAGCTGCCTGCAGG | |
| ITR-28 | AGGAACCCCTAGTGATGGAGTTGGCCACTCCCTCTCTGCGCGCTCG | 25 |
| Right | CTCGCTCACTGAGGCCGGGCGACCAAAGGTCGCCCGACGCCCGTT | |
| | TCGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCG | 26 |
| Right | CTCGCTCACTGAGGCCGGGCGACCAAAGGTCGCCCGACGCCCTTT | |
| | GGGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCG | 27 |
| Right | CTCGCTCACTGAGGCCGGGCGACCAAAGGTCGCCCGACGCCTTTG | |
| | GCGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCG | 28 |
| Right | CTCGCTCACTGAGGCCGGGCGACCAAAGGTCGCCCGACGCTTTGC | |
| | GGCCTCAGTGAGCGAGCGAGCGCGCAGCTGCCTGCAGG | |
| ITR-32 | AGGAACCCCTAGTGATGGAGTTGGCCACTCCCTCTCTGCGCGCTCG | 29 |
| Right | CTCGCTCACTGAGGCCGGGCGACCAAAGGTCGCCCGACGTTTCGG | |
| | CCTCAGTGAGCGAGCGAGCGCGCAGCTGCCTGCAGG | |
| ITR-49 | AGGAACCCCTAGTGATGGAGTTGGCCACTCCCTCTCTGCGCGCTCG | 30 |
| Right | CTCGCTCACTGAGGCCGGGCGACCAAAGGTCGCCCGACGGCCTCA | |
| | GTGAGCGAGCGAGCGCGCAGCTGCCTGCAGG | |
| ITR-50 | AGGAACCCCTAGTGATGGAGTTGGCCACTCCCTCTCTGCGCGCTCG | 31 |
| right | CTCGCTCACTGAGGCCGGGCGACCAAAGGTCGCCCGACGCCCGGG | |
| | CGGCCTCAGTGAGCGAGCGAGCGCGCAGCTGCCTGCAGG | |

TABLE 11B
Exemplary modified left ITRs. These exemplary modified left ITRs
can comprise the RBE of GCGCGCTCGCTCGCTC-3′, spacer of ACTGAGGC, the spacer
complement GCCTCAGT and RBE complement (RBE′) of GAGCGAGCGAGCGCGC.

| | | |
|---|---|---|
| ITR-33 Left | CCTGCAGGCAGCTGCGCGCTCGCTCGCTCACTGAGGCCGCCCGGGAA | 32 |
| | ACCCGGGCGTGCGCCTCAGTGAGCGAGCGAGCGCGCAGAGAGGGAGT | |
| | GGCCAACTCCATCACTAGGGGTTCCT | |
| ITR-34 Left | CCTGCAGGCAGCTGCGCGCTCGCTCGCTCACTGAGGCCGTCGGGCGA | 33 |
| | CCTTTGGTCGCCCGGCCTCAGTGAGCGAGCGAGCGCGCAGAGAGGGA | |
| | GTGGCCAACTCCATCACTAGGGGTTCCT | |
| ITR-35 Left | CCTGCAGGCAGCTGCGCGCTCGCTCGCTCACTGAGGCCGCCCGGGCA | 34 |
| | AAGCCCGGGCGTCGGCCTCAGTGAGCGAGCGAGCGCGCAGAGAGGG | |
| | AGTGGCCAACTCCATCACTAGGGGTTCCT | |
| ITR-36 Left | CCTGCAGGCAGCTGCGCGCTCGCTCGCTCACTGAGGCGCCCGGGCGTC | 35 |
| | GGGCGACCTTTGGTCGCCCGGCCTCAGTGAGCGAGCGAGCGCGCAGA | |
| | GAGGGAGTGGCCAACTCCATCACTAGGGGTTCCT | |
| ITR-37 Left | CCTGCAGGCAGCTGCGCGCTCGCTCGCTCACTGAGGCAAAGCCTCAGT | 36 |
| | GAGCGAGCGAGCGCGCAGAGAGGGAGTGGCCAACTCCATCACTAGG | |
| | GGTTCCT | |
| ITR-38 Left | CCTGCAGGCAGCTGCGCGCTCGCTCGCTCACTGAGGCCGCCCGGGCA | 37 |
| | AAGCCCGGGCGTCGGGCGACTTTGTCGCCCGGCCTCAGTGAGCGAGC | |
| | GAGCGCGCAGAGAGGGAGTGGCCAACTCCATCACTAGGGGTTCCT | |
| ITR-39 Left | CCTGCAGGCAGCTGCGCGCTCGCTCGCTCACTGAGGCCGCCCGGGCA | 38 |
| | AAGCCCGGGCGTCGGGCGATTTTCGCCCGGCCTCAGTGAGCGAGCGA | |
| | GCGCGCAGAGAGGGAGTGGCCAACTCCATCACTAGGGGTTCCT | |
| ITR-40 Left | CCTGCAGGCAGCTGCGCGCTCGCTCGCTCACTGAGGCCGCCCGGGCA | 39 |
| | AAGCCCGGGCGTCGGGCGTTTCGCCCGGCCTCAGTGAGCGAGCGAGC | |
| | GCGCAGAGAGGGAGTGGCCAACTCCATCACTAGGGGTTCCT | |
| ITR-41 Left | CCTGCAGGCAGCTGCGCGCTCGCTCGCTCACTGAGGCCGCCCGGGCA | 40 |
| | AAGCCCGGGCGTCGGGCTTTGCCCGGCCTCAGTGAGCGAGCGAGCGC | |
| | GCAGAGAGGGAGTGGCCAACTCCATCACTAGGGGTTCCT | |
| ITR-42 Left | CCTGCAGGCAGCTGCGCGCTCGCTCGCTCACTGAGGCCGCCCGGGAA | 41 |
| | ACCCGGGCGTCGGGCGACCTTTGGTCGCCCGGCCTCAGTGAGCGAGC | |
| | GAGCGCGCAGAGAGGGAGTGGCCAACTCCATCACTAGGGGTTCCT | |
| ITR-43 Left | CCTGCAGGCAGCTGCGCGCTCGCTCGCTCACTGAGGCCGCCCGGAAA | 42 |
| | CCGGGCGTCGGGCGACCTTTGGTCGCCCGGCCTCAGTGAGCGAGCGA | |
| | GCGCGCAGAGAGGGAGTGGCCAACTCCATCACTAGGGGTTCCT | |
| ITR-44 Left | CCTGCAGGCAGCTGCGCGCTCGCTCGCTCACTGAGGCCGCCCGAAAC | 43 |
| | GGGCGTCGGGCGACCTTTGGTCGCCCGGCCTCAGTGAGCGAGCGAGC | |
| | GCGCAGAGAGGGAGTGGCCAACTCCATCACTAGGGGTTCCT | |
| ITR-45 Left | CCTGCAGGCAGCTGCGCGCTCGCTCGCTCACTGAGGCCGCCCAAAGG | 44 |
| | GCGTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-46 Left | CCTGCAGGCAGCTGCGCGCTCGCTCGCTCACTGAGGCCGCCAAAGGC | 45 |
| | GTCGGGCGACCTTTGGTCGCCCGGCCTCAGTGAGCGAGCGAGCGCGC | |
| | AGAGAGGGAGTGGCCAACTCCATCACTAGGGGTTCCT | |
| ITR-47 Left | CCTGCAGGCAGCTGCGCGCTCGCTCGCTCACTGAGGCCGCAAAGCGT | 46 |
| | CGGGCGACCTTTGGTCGCCCGGCCTCAGTGAGCGAGCGAGCGCGCAG | |
| | AGAGGGAGTGGCCAACTCCATCACTAGGGGTTCCT | |
| ITR-48 Left | CCTGCAGGCAGCTGCGCGCTCGCTCGCTCACTGAGGCCGAAACGTCG | 47 |
| | GGCGACCTTTGGTCGCCCGGCCTCAGTGAGCGAGCGAGCGCGCAGAG | |
| | AGGGAGTGGCCAACTCCATCACTAGGGGTTCCT | |

According to some embodiments, a ceDNA vector for expression of peptides (e.g., antigens) comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. According to some embodiments, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes According to some ITR relative to the WT-ITR are not reflected in the other ITR; or alternatively, where the asymmetric ITRs have a modified asymmetric ITR pair can have a different sequence and different three-dimensional shape with respect to each other. Exemplary asymmetric ITRs in the ceDNA vector for expression of peptides (e.g., antigens) and for use to generate a ceDNA-plasmid are shown in Table 11A and 11B.

In an alternative embodiment, a ceDNA vector for expression of peptides (e.g., antigens) comprises two symmetrical mod-ITRs—that is, both ITRs have the same sequence, but are reverse complements (inverted) of each other. According to some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AA CG between the G and A to result in the sequence ATCGAACGATCG. The corresponding 3′ ITR sense strand is CGATCGAT (the reverse complement of ATCGATCG) with an addition of CGTT (i.e. the reverse complement of AACG) between the T and C to result in the sequence CGATCGTTCGAT (the reverse complement of ATCGAACGATCG).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′mod-ITR and the 3′ mod-ITR have the same or symmetrical three-dimensional spatial organization, they are encompassed for use herein as a modified ITR pair.

According to some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG, and modified 3′ ITR as CGATCGTTCGAT (i.e., the reverse complement of ATCGAACGATCG), these modified ITRs would still be symmetrical if, for example, the 5′ ITR had the sequence of ATCGAACCATCG, where G in the addition is modified to C, and the substantially symmetrical 3′ ITR has the sequence of CGATCGTTCGAT, without the corresponding modification of the T in the addition to a. According to some embodiments, such a modified ITR pair are substantially symmetrical as the modified ITR pair has symmetrical stereochemistry.

Table 12 shows exemplary symmetric modified ITR pairs (i.e., a left modified ITRs and the symmetric right modified ITR) for use in a ceDNA vector for expression of antigens, or immunogenic peptides. The bold (red) portion of the sequences identify partial ITR sequences (i.e., sequences of A-A′, C-C′ and B-B′ loops), also shown in FIGS. 31A-46B. These exemplary modified ITRs can comprise the RBE of GCGCGCTCGCTCGCTC-3′, spacer of ACTGAGGC, the spacer complement and RBE′ (i.e., complement to RBE) of GAGCGAGCGAGCGCGC.

TABLE 12
Exemplary symmetric modified ITR pairs in a ceDNA vector for expression of
antigens, or immunogenic peptides.

| LEFT modified ITR | | Symmetric RIGHT modified ITR | |
|---|---|---|---|
| (modified 5′ ITR) | | (modified 3′ ITR) | |
| SEQ ID NO: 32 | CCTGCAGGCAGCTGCGCGCTCG | SEQ ID NO: 15 | AGGAACCCCTAGTGATGG |
| (ITR-33 left) | CTCGCTCACTGAGGCCGCCCG | (ITR-18, right) | AGTTGGCCACTCCCTCTCT |
| | GGAAACCCGGGCGTGCGCCTC | | GCGCGCTCGCTCGCTCA |
| | AGTGAGCGAGCGAGCGCGCAG | | CTGAGGCGCACGCCCGG |
| | AGAGGGAGTGGCCAACTCCATC | | GTTTCCCGGGCGGCCTC |
| | ACTAGGGGTTCCT | | AGTGAGCGAGCGAGCGC |
| | | | GCAGCTGCCTGCAGG |
| SEQ ID NO: 33 | CCTGCAGGCAGCTGCGCGCTCG | SEQ ID NO: 48 | AGGAACCCCTAGTGATGG |
| (ITR-34 left) | CTCGCTCACTGAGGCCGTCGG | (ITR-51, right) | AGTTGGCCACTCCCTCTCT |
| | GCGACCTTTGGTCGCCCGGCC | | GCGCGCTCGCTCGCTCA |
| | TCAGTGAGCGAGCGAGCGCGC | | CTGAGGCCGGGCGACCA |
| | AGAGAGGGAGTGGCCAACTCCA | | AAGGTCGCCCGACGGCC |
| | TCACTAGGGGTTCCT | | TCAGTGAGCGAGCGAGC |
| | | | GCGCAGCTGCCTGCAGG |
| SEQ ID NO: 34 | CCTGCAGGCAGCTGCGCGCTCG | SEQ ID NO: 16 | AGGAACCCCTAGTGATGG |
| (ITR-35 left) | CTCGCTCACTGAGGCCGCCCG | (ITR-19, right) | AGTTGGCCACTCCCTCTCT |
| | GGCAAAGCCCGGGCGTCGGCC | | GCGCGCTCGCTCGCTCA |
| | TCAGTGAGCGAGCGAGCGCGC | | CTGAGGCCGACGCCCGG |
| | AGAGAGGGAGTGGCCAACTCCA | | GCTTTGCCCGGGCGGCC |
| | TCACTAGGGGTTCCT | | TCAGTGAGCGAGCGAGC |
| | | | GCGCAGCTGCCTGCAGG |
| SEQ ID NO: 35 | CCTGCAGGCAGCTGCGCGCTCG | SEQ ID NO: 17 | AGGAACCCCTAGTGATGG |
| (ITR-36 left) | CTCGCTCACTGAGGCGCCCGG | (ITR-20, right) | AGTTGGCCACTCCCTCTCT |
| | GCGTCGGGCGACCTTTGGTCG | | GCGCGCTCGCTCGCTCA |
| | CCCGGCCTCAGTGAGCGAGCG | | CTGAGGCCGGGCGACCA |
| | AGCGCGCAGAGAGGGAGTGGC | | AAGGTCGCCCGACGCCC |
| | CAACTCCATCACTAGGGGTTCCT | | GGGCGCCTCAGTGAGCG |
| | | | AGCGAGCGCGCAGCTGC |
| | | | CTGCAGG |
| SEQ ID NO: 36 | CCTGCAGGCAGCTGCGCGCTCG | SEQ ID NO: 18 | AGGAACCCCTAGTGATGG |
| (ITR-37 left) | CTCGCTCACTGAGGCAAAGCC | (ITR-21, right) | AGTTGGCCACTCCCTCTCT |
| | TCAGTGAGCGAGCGAGCGCGC | | GCGCGCTCGCTCGCTCA |
| | AGAGAGGGAGTGGCCAACTCCA | | CTGAGGCTTTGCCTCAG |
| | TCACTAGGGGTTCCT | | TGAGCGAGCGAGCGCGC |
| | | | AGCTGCCTGCAGG |
| SEQ ID NO: 37 | CCTGCAGGCAGCTGCGCGCTCG | SEQ ID NO: 19 | AGGAACCCCTAGTGATGG |
| (ITR-38 left) | CTCGCTCACTGAGGCCGCCCG | (ITR-22 right) | AGTTGGCCACTCCCTCTCT |
| | GGCAAAGCCCGGGCGTCGGGC | | GCGCGCTCGCTCGCTCA |
| | GACTTTGTCGCCCGGCCTCAG | | CTGAGGCCGGGCGACAA |
| | TGAGCGAGCGAGCGCGCAGAG | | AGTCGCCCGACGCCCGG |
| | AGGGAGTGGCCAACTCCATCAC | | GCTTTGCCCGGGCGGCC |
| | TAGGGGTTCCT | | TCAGTGAGCGAGCGAGC |
| | | | GCGCAGCTGCCTGCAGG |
| SEQ ID NO: 38 | CCTGCAGGCAGCTGCGCGCTCG | SEQ ID NO: 20 | AGGAACCCCTAGTGATGG |
| (ITR-39 left) | CTCGCTCACTGAGGCCGCCCG | (ITR-23, right) | AGTTGGCCACTCCCTCTCT |
| | GGCAAAGCCCGGGCGTCGGGC | | GCGCGCTCGCTCGCTCA |
| | GATTTTCGCCCGGCCTCAGTG | | CTGAGGCCGGGCGAAAA |
| | AGCGAGCGAGCGCGCAGAGAG | | TCGCCCGACGCCCGGGC |
| | GGAGTGGCCAACTCCATCACTA | | TTTGCCCGGGCGGCCTC |
| | GGGGTTCCT | | AGTGAGCGAGCGAGCGC |
| | | | GCAGCTGCCTGCAGG |
| SEQ ID NO: 39 | CCTGCAGGCAGCTGCGCGCTCG | SEQ ID NO: 21 | AGGAACCCCTAGTGATGG |
| (ITR-40 left) | CTCGCTCACTGAGGCCGCCCG | (ITR-24, right) | AGTTGGCCACTCCCTCTCT |
| | GGCAAAGCCCGGGCGTCGGGC | | GCGCGCTCGCTCGCTCA |
| | GTTTCGCCCGGCCTCAGTGAG | | CTGAGGCCGGGCGAAAC |
| | CGAGCGAGCGCGCAGAGAGGG | | GCCCGACGCCCGGGCTT |
| | AGTGGCCAACTCCATCACTAGG | | TGCCCGGGCGGCCTCAG |
| | GGTTCCT | | TGAGCGAGCGAGCGCGC |
| | | | AGCTGCCTGCAGG |
| SEQ ID NO: 40 | CCTGCAGGCAGCTGCGCGCTCG | SEQ ID NO: 22 | AGGAACCCCTAGTGATGG |
| (ITR-41 left) | CTCGCTCACTGAGGCCGCCCG | (ITR-25 right) | AGTTGGCCACTCCCTCTCT |
| | GGCAAAGCCCGGGCGTCGGGC | | GCGCGCTCGCTCGCTCA |
| | TTTGCCCGGCCTCAGTGAGCG | | CTGAGGCCGGGCAAAGC |
| | AGCGAGCGCGCAGAGAGGGAG | | CCGACGCCCGGGCTTTG |
| | TGGCCAACTCCATCACTAGGGGT | | CCCGGGCGGCCTCAGTG |
| | TCCT | | AGCGAGCGAGCGCGCAG |
| | | | CTGCCTGCAGG |
| SEQ ID NO: 41 | CCTGCAGGCAGCTGCGCGCTCG | SEQ ID NO: 23 | AGGAACCCCTAGTGATGG |
| (ITR-42 left) | CTCGCTCACTGAGGCCGCCCG | (ITR-26 right) | AGTTGGCCACTCCCTCTCT |
| | GGAAACCCGGGCGTCGGGCGA | | GCGCGCTCGCTCGCTCA |
| | CCTTTGGTCGCCCGGCCTCAG | | CTGAGGCCGGGCGACCA |
| | TGAGCGAGCGAGCGCGCAGAG | | AAGGTCGCCCGACGCCC |
| | AGGGAGTGGCCAACTCCATCAC | | GGGTTTCCCGGGCGGCC |
| | TAGGGGTTCCT | | TCAGTGAGCGAGCGAGC |
| | | | GCGCAGCTGCCTGCAGG |
| SEQ ID NO: 42 | CCTGCAGGCAGCTGCGCGCTCG | SEQ ID NO: 24 | AGGAACCCCTAGTGATGG |
| (ITR-43 left) | CTCGCTCACTGAGGCCGCCCG | (ITR-27 right) | AGTTGGCCACTCCCTCTCT |
| | GAAACCGGGCGTCGGGCGACC | | GCGCGCTCGCTCGCTCA |
| | TTTGGTCGCCCGGCCTCAGTG | | CTGAGGCCGGGCGACCA |
| | AGCGAGCGAGCGCGCAGAGAG | | AAGGTCGCCCGACGCCC |
| | GGAGTGGCCAACTCCATCACTA | | GGTTTCCGGGCGGCCTC |
| | GGGGTTCCT | | AGTGAGCGAGCGAGCGC |
| | | | GCAGCTGCCTGCAGG |
| SEQ ID NO: 43 | CCTGCAGGCAGCTGCGCGCTCG | SEQ ID NO: 25 | AGGAACCCCTAGTGATGG |
| (ITR-44 left) | CTCGCTCACTGAGGCCGCCCG | (ITR-28 right) | AGTTGGCCACTCCCTCTCT |
| | AAACGGGCGTCGGGCGACCTT | | GCGCGCTCGCTCGCTCA |
| | TGGTCGCCCGGCCTCAGTGAG | | CTGAGGCCGGGCGACCA |
| | CGAGCGAGCGCGCAGAGAGGG | | AAGGTCGCCCGACGCCC |
| | AGTGGCCAACTCCATCACTAGG | | GTTTCGGGCGGCCTCAG |
| | GGTTCCT | | TGAGCGAGCGAGCGCGC |
| | | | AGCTGCCTGCAGG |
| SEQ ID NO: 44 | CCTGCAGGCAGCTGCGCGCTCG | SEQ ID NO:26 | AGGAACCCCTAGTGATGG |
| (ITR-45 left) | CTCGCTCACTGAGGCCGCCCA | (ITR-29, right) | AGTTGGCCACTCCCTCTCT |
| | AAGGGCGTCGGGCGACCTTTG | | GCGCGCTCGCTCGCTCA |
| | GTCGCCCGGCCTCAGTGAGCG | | CTGAGGCCGGGCGACCA |
| | AGCGAGCGCGCAGAGAGGGAG | | AAGGTCGCCCGACGCCC |
| | TGGCCAACTCCATCACTAGGGGT | | TTTGGGCGGCCTCAGTG |
| | TCCT | | AGCGAGCGAGCGCGCAG |
| | | | CTGCCTGCAGG |
| SEQ ID NO: 45 | CCTGCAGGCAGCTGCGCGCTCG | SEQ ID NO: 27 | AGGAACCCCTAGTGATGG |
| (ITR-46 left) | CTCGCTCACTGAGGCCGCCAA | (ITR-30, right) | AGTTGGCCACTCCCTCTCT |
| | AGGCGTCGGGCGACCTTTGGT | | GCGCGCTCGCTCGCTCA |
| | CGCCCGGCCTCAGTGAGCGAG | | CTGAGGCCGGGCGACCA |
| | CGAGCGCGCAGAGAGGGAGTG | | AAGGTCGCCCGACGCCT |
| | GCCAACTCCATCACTAGGGGTTC | | TTGGCGGCCTCAGTGAG |
| | CT | | CGAGCGAGCGCGCAGCT |
| | | | GCCTGCAGG |
| SEQ ID NO: 46 | CCTGCAGGCAGCTGCGCGCTCG | SEQ ID NO: 28 | AGGAACCCCTAGTGATGG |
| (ITR-47, left) | CTCGCTCACTGAGGCCGCAAA | (ITR-31, right) | AGTTGGCCACTCCCTCTCT |
| | GCGTCGGGCGACCTTTGGTCG | | GCGCGCTCGCTCGCTCA |
| | CCCGGCCTCAGTGAGCGAGCG | | CTGAGGCCGGGCGACCA |
| | AGCGCGCAGAGAGGGAGTGGC | | AAGGTCGCCCGACGCTT |
| | CAACTCCATCACTAGGGGTTCCT | | TGCGGCCTCAGTGAGCG |
| | | | AGCGAGCGCGCAGCTGC |
| | | | CTGCAGG |
| SEQ ID NO: 47 | CCTGCAGGCAGCTGCGCGCTCG | SEQ ID NO: 29 | AGGAACCCCTAGTGATGG |
| (ITR-48, left) | CTCGCTCACTGAGGCCGAAAC | (ITR-32 right) | AGTTGGCCACTCCCTCTCT |
| | GTCGGGCGACCTTTGGTCGCC | | GCGCGCTCGCTCGCTCA |
| | CGGCCTCAGTGAGCGAGCGAG | | CTGAGGCCGGGCGACCA |
| | CGCGCAGAGAGGGAGTGGCCA | | AAGGTCGCCCGACGTTT |
| | ACTCCATCACTAGGGGTTCCT | | CGGCCTCAGTGAGCGAG |
| | | | CGAGCGCGCAGCTGCCT |
| | | | GCAGG |

According to some embodiments, a ceDNA vector for expression of peptides (e.g., antigens) comprising an asymmetric ITR pair can comprise an ITR with a modification corresponding to any of the modifications in ITR sequences or ITR partial sequences shown in any one or more of Tables 11A-11B herein, or the sequences shown in FIG. 7A-7B of International Patent Application No. PCT/US2018/064242, filed Dec. 6, 2018, which is incorporated herein in its entirety, or disclosed in Tables 2, 3, 4, 5, 6, 7, 8, 9 or 10A-10B of International Patent Application No. PCT/US18/49996 filed Sep. 7, 2018 which is incorporated herein in its entirety by reference.

Exemplary ceDNA Vectors

As described above, the present disclosure relates to recombinant ceDNA expression vectors and ceDNA vectors that encode peptides (e.g., antigens) comprising any one of: an asymmetrical ITR pair, a symmetrical ITR pair, or substantially symmetrical ITR pair as described above. In certain embodiments, the disclosure relates to recombinant ceDNA vectors for expression of peptides (e.g., antigens) having flanking ITR sequences and a transgene, where the ITR sequences are asymmetrical, symmetrical or substantially symmetrical relative to each other as defined herein, and the ceDNA further comprises a nucleic acid sequence of interest (for example an expression cassette comprising the nucleic acid of a transgene) located between the flanking ITRs, wherein said nucleic acid molecule is devoid of viral capsid protein coding sequences.

The ceDNA expression vector for expression of peptides (e.g., antigens) may be any ceDNA vector that can be conveniently subjected to recombinant DNA procedures including nucleic acid sequence(s) as described herein, provided at least one ITR is altered. The ceDNA vectors for expression of peptides (e.g., antigens) of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “transgene”, “nucleic acid sequence” and “heterologous nucleic acid sequence” are synonymous, and encode peptides (e.g., antigens) as described herein.

Referring now to FIGS. 1A-1G of International Publication No. WO/2019/051255, incorporated by reference in its entirety herein, schematics of the functional components of two non-limiting plasmids useful in making a ceDNA vector for expression of peptides (e.g., antigens) are shown. FIG. 1A, 1B, 1D, 1F show the construct of ceDNA vectors or the corresponding sequences of ceDNA plasmids for expression of antigens, or immunogenic peptides. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors for expression of peptides (e.g., antigens) are capsid-free and can be obtained from a plasmid encoding in this order: a first ITR, an expressible transgene (protein or nucleic acid) and a second ITR, where the first and second ITR sequences are asymmetrical, symmetrical or substantially symmetrical relative to each other as defined herein. According to some embodiments, the expressible transgene cassette includes, as needed: an enhancer/promoter, one or more homology arms, a donor sequence, a post-transcription regulatory element (e.g., WPRE, e.g., SEQ ID NO: 67)), and a polyadenylation and termination signal (e.g., BGH polyA, e.g., SEQ ID NO: 68).

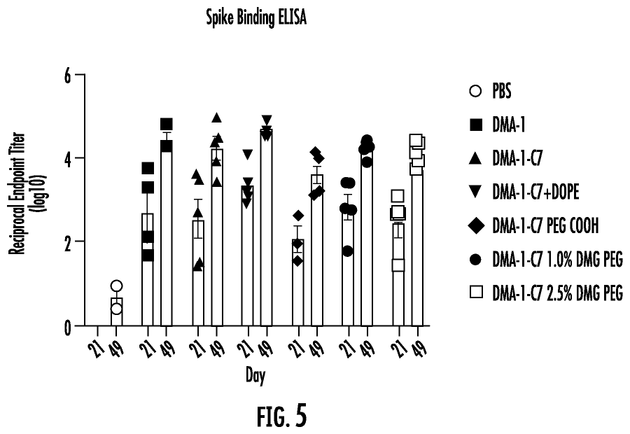

FIG. 5 of International Publication No. WO/2019/051255, incorporated by reference in its entirety herein, is a gel confirming the production of ceDNA from multiple plasmid constructs using the method described in the Examples. The ceDNA is confirmed by a characteristic band pattern in the gel, as discussed with respect to FIG. 4A of International Publication No. WO/2019/051255, incorporated by reference in its entirety herein.

### Regulatory Elements

The ceDNA vectors for expression of peptides (e.g., antigens) as described herein comprising an asymmetric ITR pair or symmetric ITR pair as defined herein, can further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer.

According to some embodiments, sequences of various cis-regulatory elements can be selected from any of those disclosed in International Application No. PCT/US2021/023891, filed on Mar. 24, 2021, the contents of which are incorporated by reference in its entirety herein.

In embodiments, the second nucleic acid sequence includes a regulatory sequence, and a nucleic acid sequence encoding a nuclease. In certain embodiments the gene regulatory sequence is operably linked to the nucleic acid sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleic acid sequence encoding the nuclease(s) of the present disclosure. In certain embodiments, the second nucleic acid sequence includes an intron sequence linked to the 5′ terminus of the nucleic acid sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleic acid sequence includes an intron sequence upstream of the nucleic acid sequence encoding a nuclease, wherein the intron includes one or more nuclease cleavage site(s), and wherein the promoter is operably linked to the nucleic acid sequence encoding the nuclease.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 80) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 81 or SEQ ID NO: 155), a CAG promoter, a human alpha 1-antitypsin (HAAT) promoter (e.g., SEQ ID NO: 82), and the like. In certain embodiments, these promoters are altered at their downstream intron containing end to include one or more nuclease cleavage sites. In certain embodiments, the DNA containing the nuclease cleavage site(s) is foreign to the promoter DNA.

According to some embodiments, a promoter may also be a promoter from a human gene such as human ubiquitin C (hUbC), human actin, human myosin, human hemoglobin, human muscle creatine, or human metallothionein.

According to some embodiments, the promoter is a tissue-specific promoter. According to further embodiments, the tissue-specific promoter is a liver specific promoter. According to some embodiments, the antigen, or immunogenic protein, is targeted to the liver and/or produced in the liver by the liver specific promoter.

Any liver specific promoter known in the art is contemplated for use in the present disclosure. According to some embodiments, the liver specific promoter is selected from, but not limited to, human alpha 1-antitypsin (HAAT), natural or synthetic. According to some embodiments, delivery to the liver can be achieved using endogenous ApoE specific targeting of the composition comprising a ceDNA vector to hepatocytes via the low density lipoprotein (LDL) receptor present on the surface of the hepatocyte.

Non-limiting examples of suitable promoters for use in accordance with the present disclosure include, but are not limited to, any of the following: the CAG promoter, the EF1a promoter, IE2 promoter and the rat EF1-α promoter, mEF1 promoter, or 1E1 promoter fragment.

According to some embodiments, a promoter can be selected from any promoter sequence disclosed in International Application No. PCT/US2021/023891, filed on Mar. 24, 2021, the contents of which are incorporated by reference in its entirety herein.

### Polyadenylation Sequences:

A sequence encoding a polyadenylation sequence can be included in the ceDNA vector for expression of peptides (e.g., antigens) to stabilize an mRNA expressed from the ceDNA vector, and to aid in nuclear export and translation. According to some embodiments, the ceDNA vector does not include a polyadenylation sequence. In other embodiments, the ceDNA vector for expression of peptides (e.g., antigens) includes at least 1, at least 2, at least 3, at least 4, at least 5, at least 10, at least 15, at least 20, at least 25, at least 30, at least 40, least 45, at least 50 or more adenine dinucleotides.

According to some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include any poly-adenylation sequence known in the art or a variation thereof. Some expression cassettes can also include SV40 late polyA signal upstream enhancer (USE) sequence. According to some embodiments, a USE sequence can be used in combination with SV40 pA or heterologous poly-A signal. PolyA sequences are located 3′ of the transgene encoding the antigens, or immunogenic peptides.

According to some embodiments, a polyadenylation sequence can be selected from any polyadenylation sequence disclosed in International Application No. PCT/US2021/023891, filed on Mar. 24, 2021, the contents of which are incorporated by reference in its entirety herein.

The expression cassettes can also include a post-transcriptional element to increase the expression of a transgene. According to some embodiments, Woodchuck Hepatitis Virus (WHP) posttranscriptional regulatory element (WPRE) is used to increase the expression of a transgene.

Other posttranscriptional processing elements such as the post-transcriptional element from the thymidine kinase gene of herpes simplex virus, or hepatitis B virus (HBV) can be used.

According to some embodiments, a posttranscritptional regulatory element can be selected from any posttranscriptional regulatory element sequence disclosed in International Application No. PCT/US2021/023891, filed on Mar. 24, 2021, the contents of which are incorporated by reference in its entirety herein.

According to some embodiments, one or more nucleic acid sequences that encode an antigen, or immunogenic protein, can also encode a secretory sequence so that the protein is directed to the Golgi Apparatus and Endoplasmic Reticulum and folded into the correct conformation by chaperone molecules as it passes through the ER and out of the cell. Exemplary secretory sequences include, but are not limited to VH-02 and VK-A26) and Igx signal sequence, as well as a Glue secretory signal that allows the tagged protein to be secreted out of the cytosol, TMD-ST secretory sequence, that directs the tagged protein to the golgi.

According to some embodiments, a secretory sequence can be selected from any secretory sequence disclosed in International Application No. PCT/US2021/023891, filed on Mar. 24, 2021, the contents of which are incorporated by reference in its entirety herein.

### Nuclear Localization Sequences

According to some embodiments, the ceDNA vector for expression of peptides (e.g., antigens) comprises one or more nuclear localization sequences (NLSs), for example, 1, 2, 3, 4, 5, 6, 7, 8, 9, 10, or more NLSs. According to some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present According to some or more copies.

According to some embodiments, a NLS can be selected from any NLS disclosed in International Application No. PCT/US2021/023891, filed on Mar. 24, 2021, the contents of which are incorporated by reference in its entirety herein.

### V. Method of Production of a ceDNA Vector

### Production in General

Certain methods for the production of a ceDNA vector for expression of peptides (e.g., antigens) comprising an asymmetrical ITR pair or symmetrical ITR pair as defined herein is described in section IV of International application PCT/US18/49996 filed Sep. 7, 2018, which is incorporated herein in its entirety by reference. According to some embodiments, a ceDNA vector for expression of peptides (e.g., antigens) as disclosed herein can be produced using insect cells, as described herein. In alternative embodiments, a ceDNA vector for expression of peptides (e.g., antigens) as disclosed herein can be produced synthetically and according to some embodiments, in a cell-free method, as disclosed in International Application PCT/US19/14122, filed Jan. 18, 2019, which is incorporated herein in its entirety by reference.

As described herein, according to some embodiments, a ceDNA vector for expression of peptides (e.g., antigens) can be obtained, for example, by the process comprising the steps of: a) incubating a population of host cells (e.g., insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g., AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the disclosure provides for use of host cell lines that have stably integrated the DNA vector polynucleotide expression template (ceDNA template) into their own genome in production of the non-viral DNA vector, e.g., as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

According to some embodiments, the host cells used to make the ceDNA vectors for expression of peptides (e.g., antigens) as described herein are insect cells, and baculovirus is used to deliver both the polynucleotide that encodes Rep protein and the non-viral DNA vector polynucleotide expression construct template for ceDNA, e.g., as described in Example 1. According to some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. According to some embodiments, cells are grown and harvested a sufficient time after baculoviral infection to produce ceDNA vectors but before most cells start to die due to the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. According to some embodiments, ceDNA vectors are purified as DNA molecules. In another embodiment, the ceDNA vectors are purified as exosomes or microparticles.

The presence of the ceDNA vector for expression of peptides (e.g., antigens) can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA.

According to some embodiments, the ceDNA is synthetically produced in a cell-free environment.

ceDNA Plasmid

A ceDNA-plasmid is a plasmid used for later production of a ceDNA vector for expression of peptides (e.g., antigens) as described herein. According to some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. According to some embodiments, the expression cassette flanked by the ITRs comprises a cloning site for introducing an exogenous sequence. The expression cassette replaces the rep and cap coding regions of the AAV genomes.

According to some aspects, a ceDNA vector for expression of peptides (e.g., antigens) is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs have the same modifications (i.e., they are inverse complement or symmetric relative to each other).

In a further embodiment, the ceDNA-plasmid system is devoid of viral capsid protein coding sequences (i.e., it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present disclosure can be generated using natural nucleic acid sequences of the genomes of any AAV serotypes well known in the art. According to some embodiments, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at the address oesys.springer.de/viruses/database/mkchapter.asp?virID=42.04.) (note -references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. According to some embodiments, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAV0) vector for expression of peptides (e.g., antigens) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

Exemplary Method of Making the ceDNA Vectors from ceDNA Plasmids

Methods for making capsid-less ceDNA vectors for expression of peptides (e.g., antigens) are also provided herein, notably a method with a sufficiently high yield to provide sufficient vector for in vivo experiments.

According to some embodiments, a method for the production of a ceDNA vector for expression of peptides (e.g., antigens) comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### Cell Lines

Host cell lines used in the production of a ceDNA vector for expression of peptides (e.g., antigens) can include insect cell lines derived from Spodopterafrugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, Hep1A, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

Isolating and Purifying ceDNA Vectors

Examples of the process for obtaining and isolating ceDNA vectors are described in FIGS. 4A-4E of International Publication No. WO/2019/051255, incorporated by reference in its entirety herein. ceDNA-vectors for expression of peptides (e.g., antigens) used as a priming vaccine in the prime-boost compositions and methods described herein, can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode peptides (e.g., antigens) or plasmids encoding one or more REP proteins.

According to some aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for expression of peptides (e.g., antigens) used as a priming vaccine in the prime-boost compositions and methods described herein, are described herein. Expression constructs used for generating a ceDNA vector for expression of peptides (e.g., antigens) as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for expression of peptides (e.g., antigens) can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for expression of peptides (e.g., antigens) as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The capsid-free AAV vector is then recovered by, e.g., precipitation.

According to some embodiments, ceDNA vectors for expression of peptides (e.g., antigens) can also be purified in the form of exosomes, or microparticles. It is known in the art that many cell types release not only soluble proteins, but also complex protein/nucleic acid cargoes via membrane microvesicle shedding (Cocucci et al., 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the disclosure herein relates to methods of purifying ceDNA vectors from host cell lines that have stably integrated a ceDNA construct into their own genome. According to some embodiments, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs.

### VI. Administration

### Methods for Heterologous Prime-Boost Immunization

Multi-dose immunization, for therapy or for disease prevention, has been reported to be often more effective than single-dose immunization. It is generally believed that generating a high number of antigen-specific memory CD8⁺ T cells following vaccination is a desirable goal for vaccine design against a variety of animal and human diseases, because this number strongly correlates with host immunization and protection. One approach to generate these high numbers of cells is to use prime-boost immunization, which relies on the re-stimulation of antigen-specific immune cells following primary memory formation. In such a process, there is a “priming” composition which is administered to the subject first and a “boosting” composition which is subsequently administered one or more times.

The disclosure further contemplates multiple administrations of one of the compositions (the priming vaccine) followed by multiple administrations of the other composition (the boosting). In one embodiment, the priming composition is administered to the subject at least once or multiple times prior to administration of the boosting composition. Thereafter, the boosting composition is subsequently administered to the subject at least once or multiple times. It is widely believed that boosting of immune responses by vaccines results in generation of larger numbers of effector cells required for mediating protection against pathogens at the time of infection.

According to aspects of the disclosure, the methods described herein employ heterologous prime-boost immunization, or the administration of the an antigen or immunogenic peptide using two different modalities or platforms. According to embodiments, such an approach advantageously elicits improved immune responses in subjects. According to some embodiments, improved immune responses resulting from the described heterologous prime-boost immunization include, improved memory responses, which include, but are not limited to, a higher magnitude of CD8⁺ T cell responses, a broadening of T cell epitopes recognized by the immune system, and an increase in polyfunctionality of T cells. According to some embodiments the higher magnitude of CD8⁺ T cell response can be an increase of at least 20% or at least 25% or at least 30% or at least 50% or at least 75% or at least 100% or at least 150% or at least 200% or at least 250% or at least 300% versus single dose administration or versus a homologous prime-boost regimen. According to some embodiments, a heterologous prime-boost strategy described herein, wherein a ceDNA platform is used as a priming vaccine, can result in synergistic enhancement of immune response. According to further embodiments, synergistic enhancement of the immune response is seen in an increased number of antigen-specific T cells, the length of the immune memory response, and the magnitude of the immune memory response.

According to some embodiments, the disclosure provides methods of inducing an immune response against a first peptide and a second peptide in a subject, comprising administering a priming vaccine comprising a deoxyribonucleic acid (DNA) DNA to the subject, wherein the DNA encodes a first peptide; and administering a boosting vaccine comprising (i) a ribonucleic acid (RNA), or (ii) a second peptide to the subject, wherein the RNA encodes the second peptide, thereby inducing the immune response against the first peptide and the second peptide in the subject. Also provided are vaccine regimens, comprising a priming vaccine comprising a deoxyribonucleic acid (DNA) DNA, wherein the DNA encodes a first peptide; and a boosting vaccine comprising (i) a ribonucleic acid (RNA), or (ii) a second peptide, wherein the RNA encodes the second peptide. According to some embodiments, the first and the second peptide, are derived from a bacterial, a viral, a fungal or a parasitic infectious agent. According to some embodiments, the first and the second peptide are from the same pathogenic organism. According to some embodiments, the first and the second peptide are the same in the priming vaccine and the boosting vaccine. According to some embodiments, at least one of the epitopes of the first and the second peptides are different in the priming and the boosting vaccine.

Some embodiments disclosed herein relate to a method for inducing an immune response against a first peptide and a second peptide in a subject, the method comprising administering to the subject at least one dose of a priming vaccine comprising a ceDNA vector which encodes a first peptide; and subsequently administering to the subject at least one dose of a boosting vaccine, wherein the boosting vaccine does not comprise a ceDNA vector.

Some embodiments disclosed herein relate to a method for inducing an immune response against a first peptide and a second peptide in a subject, the method comprising administering to the subject at least one dose of a priming vaccine comprising a ceDNA vector which encodes a first peptide; and subsequently administering to the subject at least one dose of a boosting vaccine, wherein the boosting vaccine comprises a second peptide.

Some embodiments disclosed herein relate to a method for inducing an immune response against a first peptide and a second peptide in a subject, the method comprising administering to the subject at least one dose of a priming vaccine comprising a ceDNA vector which encodes a first peptide; and subsequently administering to the subject at least one dose of a boosting vaccine, wherein the boosting vaccine comprises a ribonucleic acid (RNA) encoding the second peptide.

Some embodiments disclosed herein relate to a method for inducing an immune response against a first peptide and a second peptide in a subject, the method comprising administering to the subject at least one dose of a priming vaccine comprising a ceDNA vector which encodes a first peptide; and subsequently administering to the subject at least one dose of a boosting vaccine, wherein the boosting vaccine comprises a ceDNA vector encoding the second peptide.

Some embodiments disclosed herein relate to a method for inducing an immune response against a first peptide and a second peptide in a subject, the method comprising administering to the subject at least one dose of a priming vaccine comprising a ribonucleic acid (RNA) encoding the first peptide; and subsequently administering to the subject at least one dose of a boosting vaccine, wherein the boosting vaccine comprises a ceDNA vector encoding the second peptide.

Some embodiments disclosed herein relate to a method for inducing an immune response against a first peptide and a second peptide in a subject, the method comprising administering to the subject at least one dose of a priming vaccine comprising a first peptide; and subsequently administering to the subject at least one dose of a boosting vaccine, wherein the boosting vaccine comprises a ceDNA vector encoding the second peptide.

In some embodiments, the first peptide and the second peptide are identical to each other. In some embodiments, amino acid sequences of the first peptide and the second peptide are homologous to each other. In some embodiments, the amino acid sequence of the first peptide exhibits at least 80%, 85%, 90%, 95%, 96%, 97%, 98%, or 99% sequence identity to the amino acid sequence of the second peptide. In some embodiments, the first and the second immunogenic peptides comprise at least one cross-reactive antigenic determinant. In some embodiments, the first and the second immunogenic peptides or antigens induce substantially the same immune response in the subject.

In some embodiments, the priming composition is administered into the subject in a single dose. In some embodiments, the priming composition is administered into the subject in multiple doses. In some embodiments, the boosting composition is administered into the subject in a single dose. In some embodiments, the boosting composition is administered into the subject in multiple doses.

In some embodiments, the priming composition and/or the boosting composition is administered to the subject for at least 2, at least 3, at least 4, at least 5, or at least 10 consecutive dosages or any number dosage therebetween. In some embodiments, the priming composition and/or the boosting composition is administered to the subject for at least 10, at least 12, at least 14, at least 16, or at least 20 consecutive dosages or any number dosage therebetween.

In some embodiments of the methods disclosed herein, the at least one dose of the priming composition and the boosting composition are administered to the subject at intervals of about 1 week, or 2, 3, 4, 5, 6, 7, or 8 or 1-2 or 2-4 or 3-4 weeks. In some embodiments of the methods disclosed herein, the at least one dose of the priming composition and the boosting composition are administered into the subject at intervals of about 4 weeks. In some embodiments of the methods disclosed herein, the at least one dose of the priming composition and the boosting composition are administered into the subject at intervals of about 6 weeks. In some embodiments of the methods disclosed herein, the at least one dose of the priming composition and the boosting composition are administered into the subject at intervals of about 8 weeks. In some embodiments of the methods disclosed herein, the at least one dose of the priming composition and the boosting composition are administered into the subject at intervals of about 10 weeks.

In some embodiments of the methods disclosed herein, the at least one dose of the priming composition and the boosting composition are administered into the subject at intervals of about 28 days. In some embodiments of the methods disclosed herein, the at least one dose of the priming composition and the boosting composition are administered into the subject at intervals of about 35 days. In some embodiments of the methods disclosed herein, the at least one dose of the priming composition and the boosting composition are administered into the subject at intervals of about 42 days. In some embodiments of the methods disclosed herein, the at least one dose of the priming composition and the boosting composition are administered into the subject at intervals of about 49 days. In some embodiments of the methods disclosed herein, the at least one dose of the priming composition and the boosting composition are administered into the subject at intervals of about 56 days. In some embodiments of the methods disclosed herein, the at least one dose of the priming composition and the boosting composition are administered into the subject at intervals of about 63 days. In some embodiments of the methods disclosed herein, the at least one dose of the priming composition and the boosting composition are administered into the subject at intervals of about 70 days or more. In some embodiments of the methods disclosed herein, the at least one dose of the priming composition and the boosting composition are administered into the subject at intervals of between 28 days and 56 days.

One of skill in the art will further appreciate that for any particular subject, specific dosage regimens can be adjusted over time according to the individual need and the professional judgment of the person administering or supervising the administration of the compositions. For example, doses may be adjusted based on clinical effects of the administered compositions such as toxic effects and/or laboratory values. Dosage regimens can be adjusted to provide the optimal desired effect. For example, as discussed above, a single dose can be administered, several divided doses can be administered over time or the dose can be proportionally reduced or increased as indicated by the exigencies of the therapeutic situation. Determining appropriate dosages and regimens for administration of the compositions disclosed herein are well-known in the relevant art and would be understood to be encompassed by the skilled artisan once provided the teachings disclosed herein.

Thus, a person of skill in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

Administration of the priming and boosting compositions disclosed herein may be carried out by any method that enables delivery of the compositions to the site of action. These methods include oral routes, intraduodenal routes, parenteral injection (comprising intravenous, subcutaneous, intramuscular, intravascular, or infusion), topical administration, and rectal administration. Infusions can be administered by drip, continuous infusion, infusion pump, metering pump, depot formulation, or any other suitable means. In some embodiments, at least one dose of the priming composition is administered intramuscularly to the subject. In some embodiments, at least one dose of the boosting composition is administered intramuscularly to the subject.

In some embodiments disclosed herein, the methods of the disclosure further include one or more subsequent boosting administrations. In some embodiments, the methods of the disclosure further include at least 2, at least 3, at least 4, at least 5, or at least 10 consecutive boosting administrations or any number administration therebetween. In some embodiments, the subsequent boosting administrations are performed in gradually increasing dosages over time. In some embodiments, the subsequent boosting administrations are performed in gradually decreasing dosages over time.

### VII. Pharmaceutical Compositions

In another aspect, pharmaceutical compositions are provided. The pharmaceutical composition comprises a ceDNA vector for expression of peptides (e.g., antigens) used as a priming vaccine in the prime-boost compositions and methods described herein, and a pharmaceutically acceptable carrier or diluent.

The ceDNA vectors for expression of peptides (e.g., antigens) as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier.

Pharmaceutical formulations disclosed herein include liquid, e.g., aqueous, solutions that may be directly administered, and lyophilized powders which may be reconstituted into solutions by adding a diluent before administration. in certain embodiments, a formulation comprising a ceDNA vector as disclosed herein, with or without at least one additional therapeutic agent, can be formulated as a lyophilizate using appropriate excipients. Lyophilization can be performed using a generic Lyophilization cycle on a commercially available lyophilizer (e.g., a VirTis Lab Scale Lyophilizer).

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

In certain embodiments, the formulation for parenteral administration can be stored in lyophilized form or in a solution. In certain embodiments, parenteral formulations generally are placed into a container having a sterile access port, for example, an intravenous solution bag or vial having a stopper pierceable by a hypodermic injection needle.

According to some aspects, the methods provided herein comprise delivering one or more ceDNA vectors for expression of peptides (e.g., antigens) used as a priming vaccine in the prime-boost compositions and methods described herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for expression of peptides (e.g., antigens) can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for expression of peptides (e.g., antigens) to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA vectors for expression of peptides (e.g., antigens) can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors for expression of peptides (e.g., antigens) as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

The ceDNA vectors for expression of peptides (e.g., antigens) in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”.

Various delivery methods known in the art or modification thereof can be used to deliver ceDNA vectors in vitro or in vivo. For example, according to some embodiments, ceDNA vectors for expression of peptides (e.g., antigens) are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. According to some embodiments, a ceDNA vector alone is directly injected as naked DNA into any one of: any one or more tissues selected from: lung, liver, kidneys, gallbladder, prostate, adrenal gland, heart, intestine, stomach, skin, thymus, cardiac muscle or skeletal muscle. According to some embodiments, a ceDNA vector is delivered by gene gun. Gold or tungsten spherical particles (1-3 μm diameter) coated with capsid-free AAV vectors can be accelerated to high speed by pressurized gas to penetrate into target tissue cells.

According to some embodiments, the ceDNA vector is formulated with a lipid delivery system, for example, liposomes as described herein. According to some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

According to some cases, a ceDNA vector for expression of peptides (e.g., antigens) is delivered by hydrodynamic injection, which is a simple and highly efficient method for direct intracellular delivery of any water-soluble compounds and particles into internal organs and skeletal muscle in an entire limb.

According to some embodiments, ceDNA vectors for expression of peptides (e.g., antigens) are delivered by ultrasound by making nanoscopic pores in membrane to facilitate intracellular delivery of DNA particles into cells of internal organs or tumors, so the size and concentration of plasmid DNA have great role in efficiency of the system. According to some embodiments, ceDNA vectors are delivered by magnetofection by using magnetic fields to concentrate particles containing nucleic acid into the target cells.

According to some embodiments, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### Exosomes

According to some embodiments, a ceDNA vector for expression of peptides (e.g., antigens) as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). According to some embodiments, exosomes with a diameter between 10 nm and 1 m, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present disclosure.

### Microparticle/Nanoparticles

According to some embodiments, a ceDNA vector for expression of peptides (e.g., antigens) as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

According to some embodiments, a lipid nanoparticle has a mean diameter between about 10 and about 1000 nm. According to some embodiments, a lipid nanoparticle has a diameter that is less than 300 nm. According to some embodiments, a lipid nanoparticle has a diameter between about 10 and about 300 nm. According to some embodiments, a lipid nanoparticle has a diameter that is less than 200 nm. According to some embodiments, a lipid nanoparticle has a diameter between about 25 and about 200 nm. According to some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for expression of peptides (e.g., antigens) as disclosed herein. For example, various delivery methods using lipid nanoparticles are described in U.S. Pat. Nos. 9,404,127, 9,006,417 and 9,518,272.

### Conjugates

According to some embodiments, a ceDNA vector for expression of peptides (e.g., antigens) as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications.

Ther. Deliv. 4(7); 791-809.

According to some embodiments, a ceDNA vector for expression of peptides (e.g., antigens) as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. According to some embodiments, a ceDNA vector for expression of peptides (e.g., antigens) as disclosed herein is conjugated to a poly(amide) polymer, for example as described by U.S. Pat. No. 8,987,377. According to some embodiments, a nucleic acid described by the disclosure is conjugated to a folic acid molecule as described in U.S. Pat. No. 8,507,455.

According to some embodiments, a ceDNA vector for expression of peptides (e.g., antigens) as disclosed herein is conjugated to a carbohydrate, for example as described in U.S. Pat. No. 8,450,467.

### Nanocapsule

Alternatively, nanocapsule formulations of a ceDNA vector for expression of peptides (e.g., antigens) as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Liposomes

The ceDNA vectors for expression of peptides (e.g., antigens) in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### Exemplary Liposome and Lipid Nanoparticle (LNP) Compositions

The ceDNA vectors for expression of peptides (e.g., antigens) in accordance with the present disclosure can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA vectors are disclosed in International Application PCT/US2018/050042, filed on Sep. 7, 2018, and International Application PCT/US2018/064242, filed on Dec. 6, 2018 which are incorporated herein in their entirety and envisioned for use in the methods and compositions for ceDNA vectors for expression of peptides (e.g., antigens) as disclosed herein.

According to some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

According to some aspects, the disclosure provides for a liposome formulation that will deliver an API with extended release or controlled release profile over a period of hours to weeks. According to some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

According to some aspects, the liposome formulation comprises sphingomyelin and one or more lipids disclosed herein. According to some aspects, the liposome formulation comprises optisomes.

According to some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

According to some aspects, the disclosure provides for a liposome formulation comprising phospholipid, cholesterol and a PEG-ylated lipid in a molar ratio of 56:38:5. According to some aspects, the liposome formulation's overall lipid content is from 2-16 mg/mL. According to some aspects, the disclosure provides for a liposome formulation comprising a lipid containing a phosphatidylcholine functional group, a lipid containing an ethanolamine functional group and a PEG-ylated lipid. According to some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively.

According to some aspects, the disclosure provides for a liposome formulation comprising a lipid containing a phosphatidylcholine functional group, cholesterol and a PEG-ylated lipid. According to some aspects, the disclosure provides for a liposome formulation comprising a lipid containing a phosphatidylcholine functional group and cholesterol. According to some aspects, the PEG-ylated lipid is PEG-2000-DSPE. According to some aspects, the disclosure provides for a liposome formulation comprising DPPG, soy PC, MPEG-DSPE lipid conjugate and cholesterol.

According to some aspects, the disclosure provides for a liposome formulation comprising one or more lipids containing a phosphatidylcholine functional group and one or more lipids containing an ethanolamine functional group. According to some aspects, the disclosure provides for a liposome formulation comprising one or more: lipids containing a phosphatidylcholine functional group, lipids containing an ethanolamine functional group, and sterols, e.g., cholesterol. According to some aspects, the liposome formulation comprises DOPC/DEPC; and DOPE.

According to some aspects, the disclosure provides for a liposome formulation further comprising one or more pharmaceutical excipients, e.g., sucrose and/or glycine.

According to some aspects, the disclosure provides for a liposome formulation that is either unilamellar or multilamellar in structure. According to some aspects, the disclosure provides for a liposome formulation that comprises multi-vesicular particles and/or foam-based particles. According to some aspects, the disclosure provides for a liposome formulation that are larger in relative size to common nanoparticles and about 150 to 250 nm in size. According to some aspects, the liposome formulation is a lyophilized powder.

According to some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. According to some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

According to some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid nanoparticles are prepared at a total lipid to ceDNA (mass or weight) ratio of from about 10:1 to 60:1. According to some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., lipid nanoparticles) are prepared at a ceDNA (mass or weight) to total lipid ratio of about 60:1. According to some embodiments, the lipid particles are prepared at a total lipid to ceDNA (mass or weight) ratio of from about 10:1 to 30:1. According to some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

According to some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

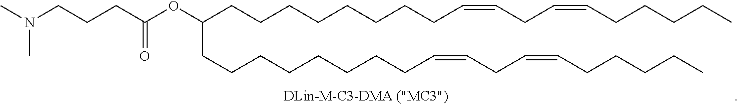 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety. According to some other embodiments, the ionizable lipid has any one of the following structures:

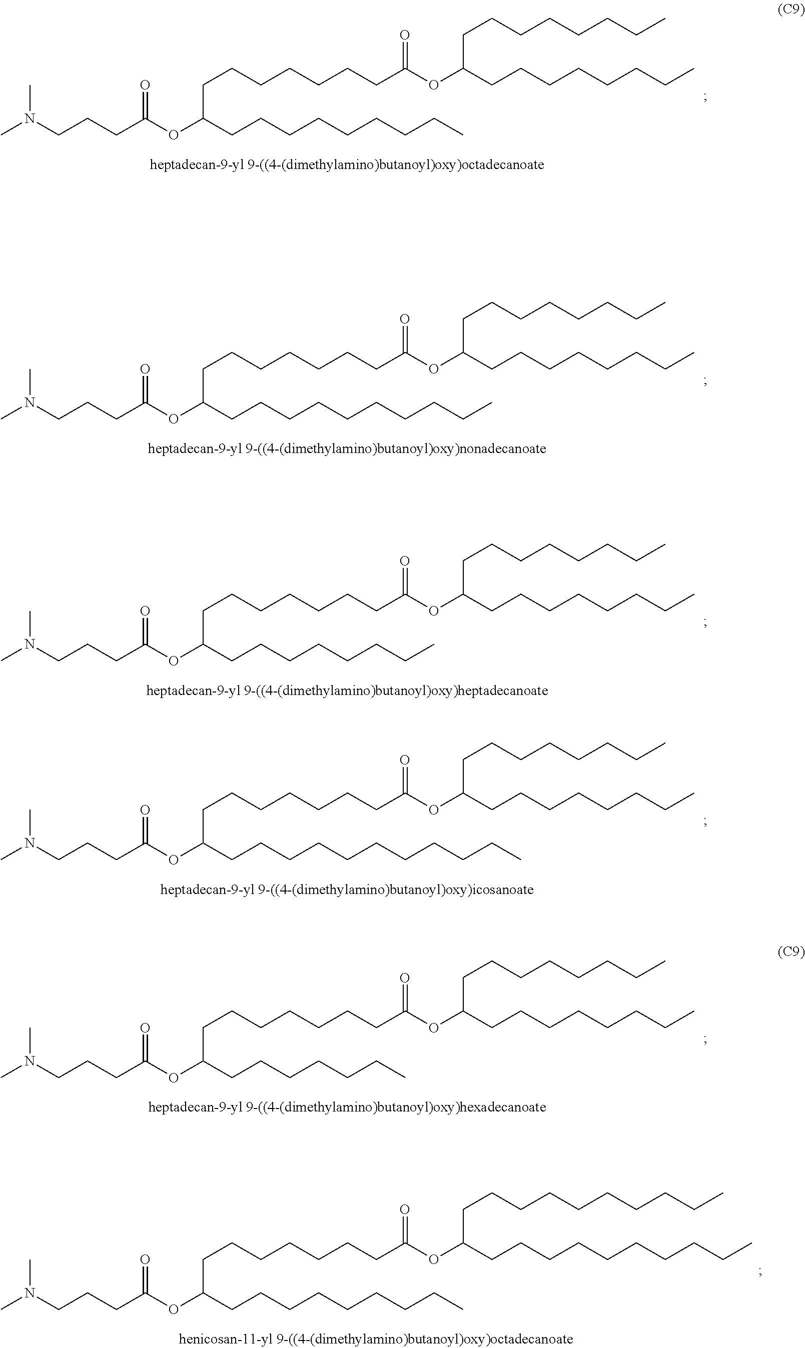

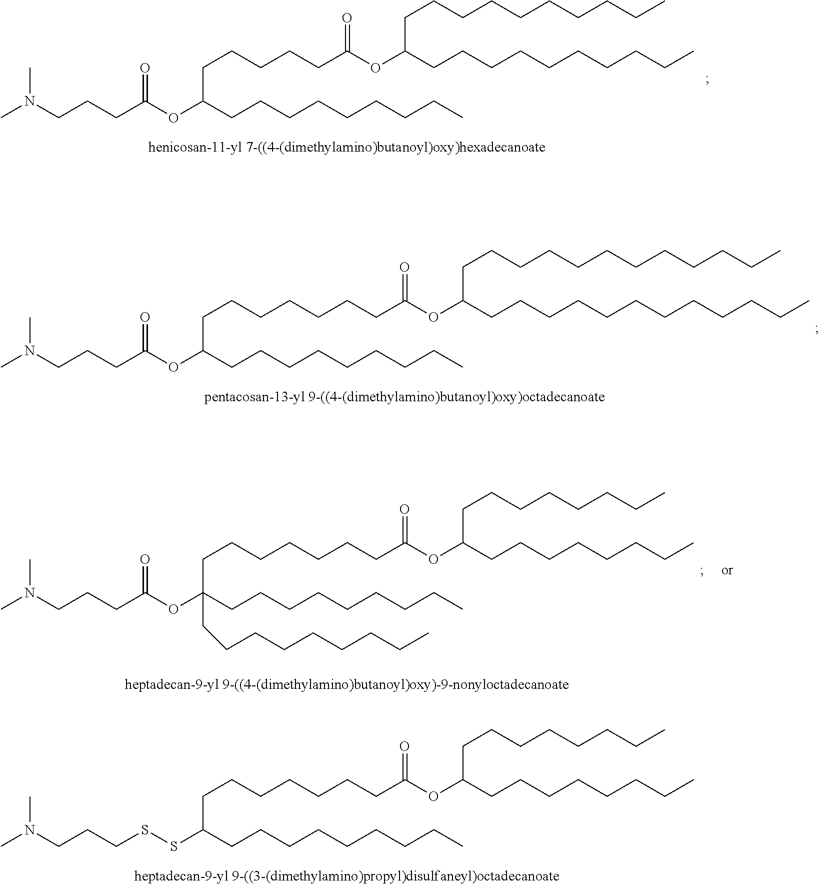

According to some embodiments, the ionizable lipid is the lipid ATX-002 as described in WO2015/074085, content of which is incorporated herein by reference in its entirety.

According to some embodiments, the ionizable lipid is (13Z,16Z)-N,N-dimethyl-3-nonyldocosa-13,16-dien-1-amine (Compound 32), as described in WO2012/040184, content of which is incorporated herein by reference in its entirety.

According to some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. According to some embodiments, ionizable lipid comprises from about 50 mol % to about 90 mol % of the total lipid present in the lipid nanoparticle.

According to some aspects, the lipid nanoparticle can further comprise a non-cationic lipid.

Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions as disclosed herein are described in International Application PCT/US2018/050042, filed on Sep. 7, 2018, and PCT/US2018/064242, filed on Dec. 6, 2018 which is incorporated herein in its entirety. Exemplary non-cationic lipids are described in International Application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

According to some embodiments, the lipid nanoparticles do not comprise any phospholipids. According to some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. According to some embodiments, such a component is 20-50% (mol) 30-40% (mol) of the total lipid content of the lipid nanoparticle.

According to some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. According to some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

According to some embodiments, a PEG-lipid is a compound as defined in US2018/0028664, the content of which is incorporated herein by reference in its entirety. According to some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. According to some examples, the PEG-lipid can be selected from the group consisting of PEG-DMG, 1,2-dimyristoyl-sn-glycero-3-phosphoethanolamine-N-[methoxy(polyethylene glycol)-2000].

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

LNP comprising ionizable lipid, sterol, non-cationic lipid, PEGylated lipid, and optionally tissue-specific targeting ligand

According to some embodiments of any of the aspects or embodiments herein, a lipid nanoparticle provided herein comprises at least one ionizable lipid as described herein, at least one sterol, at least one non-cationic lipid, and at least one PEGylated lipid. In one embodiment of any of the aspects or embodiments herein, a lipid nanoparticle provided herein consists essentially of at least one ionizable lipid as described herein, at least one sterol, at least one non-cationic lipid, and at least one PEGylated lipid. In one embodiment of any of the aspects or embodiments herein, a lipid nanoparticle provided herein consists of at least one ionizable lipid as described herein, at least one sterol, at least one non-cationic lipid, and at least one PEGylated lipid. In one embodiment of any of the aspects or embodiments herein, the molar ratio of ionizable lipid:sterol:non-cationic lipid: PEGylated lipid is about 48 (±5): 10 (±3): 41 (±5): 2 (2), e.g., about 47.5:10.0: 40.7:1.8 or about 47.5:10.0: 40.7: 3.0.

According to some embodiments of any of the aspects or embodiments herein, a lipid nanoparticle provided herein comprises at least one ionizable lipid as described herein, at least one sterol, at least one non-cationic lipid, at least one PEGylated lipid, and a tissue-specific targeting ligand. In one embodiment of any of the aspects or embodiments herein, the tissue-specific targeting ligand is GalNAc. In one embodiment of any of the aspects or embodiments herein, a lipid nanoparticle provided herein consists essentially of at least one ionizable lipid as described herein, at least one sterol, at least one non-cationic lipid, at least one PEGylated lipid, and a tissue-specific targeting ligand. In one embodiment of any of the aspects or embodiments herein, a lipid nanoparticle provided herein consists of at least one ionizable lipid as described herein, at least one sterol, at least one non-cationic lipid, at least one PEGylated lipid, and a tissue-specific targeting ligand. In one embodiment of any of the aspects or embodiments herein, the tissue-specific targeting ligand is conjugated to a PEGylated lipid to form a PEGylated lipid conjugate. In one embodiment of any of the aspects or embodiments herein, the PEGylated lipid conjugate is mono-, bi-, tri-, or tetra-antennary GalNAc-DSPE-PEG2000. In one embodiment of any of the aspects or embodiments herein, the PEGylated lipid conjugate is tetra-antennary GalNAc-DSPE-PEG2000. In one embodiment of any of the aspects or embodiments herein, the molar ratio of ionizable lipid: sterol: non-cationic lipid: PEGylated lipid: PEGylated lipid conjugate is about 48 (±5): 10 (±3): 41 (±5): 2 (±2): 1.5 (±1), e.g., 47.5:10.0: 40.2:1.8: 0.5 or 47.5:10.0: 39.5: 2.5: 0.5.

### Combinations

According to some embodiments, either of the prime-boost composition is administered in combination with one or more additional therapeutic agents, e.g., an anti-cancer therapeutic, an autoimmune therapeutic, an infectious disease therapeutic. According to some embodiments, the agent is a second antigen or immunogenic peptide, as described herein.

In some embodiments, the effect of the ceDNA and the additional agent is synergistic. The term “synergistic” or “synergy” means a more than additive effect of a combination of two or more agents compared to their individual effects. In some embodiments, synergistic activity is present when a first agent produces a detectable level of an output X, a second agent produces a detectable level of the output X, and the first and second agents together produce a more-than-additive level of the output X.

Some human tumors can be eliminated by a patient's immune system. For example, administration of a monoclonal antibody targeted to an immune “checkpoint” molecule can lead to complete response and tumor remission. A mode of action of such antibodies is through inhibition of an immune regulatory molecule that the tumors have co-opted as protection from an anti-tumor immune response. By inhibiting these “checkpoint” molecules (e.g., with an antagonistic antibody), a patient's CD8⁺ T cells may be allowed to proliferate and destroy tumor cells. For example, administration of a monoclonal antibody targeted to by way of example, without limitation, CTLA-4 or PD-1 can lead to complete response and tumor remission. The mode of action of such antibodies is through inhibition of CTLA-4 or PD-1 that the tumors have co-opted as protection from an anti-tumor immune response. By inhibiting these “checkpoint” molecules (e.g., with an antagonistic antibody), a patient's CD8⁺ T cells may be allowed to proliferate and destroy tumor cells.

Thus, the ceDNA vectors comprising a nucleic acid sequence encoding one or more tumor associated antigens provided herein can be used in combination with one or more blocking antibodies targeted to an immune “checkpoint” molecule. For instance, in some embodiments, the compositions provided herein can be used in combination with one or more blocking antibodies targeted to a molecule such as CTLA-4 or PD-1.

According to some embodiments, a ceDNA composition is administered with an adjuvant. Adjuvants include, but are not limited to, Freund's adjuvant, GM-CSF, Montanide (e.g., Montanide IMS 1312, Montanide ISA 206, Montanide ISA 50V, and Montanide ISA-51), 1018 ISS, aluminium salts, Amplivax®, AS15, BCG, CP-870,893, CpG7909, CyaA, dSLIM, flagellin or TLR5 ligands derived from flagellin, FLT3 ligand, IC30, IC31, Imiquimod (ALDARA®), resiquimod, ImuFact IMP321, Interleukins such as IL-2, IL-4, IL-7, IL-12, IL-13, IL-15, IL-21, IL-23, Interferon-a or -f, or pegylated derivatives thereof, IS Patch, ISS, ISCOMATRIX, ISCOMs, JuvImmune, LipoVac, MALP2, MF59, monophosphoryl lipid A, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, virosomes and other virus-like particles, YF-17D, VEGF trap, R848, beta-glucan, Pam3Cys, Aquila's QS21 stimulon, mycobacterial extracts and synthetic bacterial cell wall mimics, Ribi's Detox, Quil, Superfos, cyclophosphamide, sunitinib, bevacizumab, celebrex, NCX-4016, sildenafil, tadalafil, vardenafil, sorafenib, temozolomide, temsirolimus, XL-999, CP-547632, pazopanib, VEGF Trap, ZD2171, AZD2171, and anti-CTLA4 antibodies. CpG immunostimulatory oligonucleotides can be used to enhance the effects of adjuvants in a vaccine setting.

According to some embodiments, the nucleic acid sequence of the ceDNA vector further comprises a sequence that encodes an adjuvant.

Also provided herein is a pharmaceutical composition comprising the lipid nanoparticle-encapsulated insect-cell produced, or a synthetically produced ceDNA vector for expression of peptides (e.g., antigens) as described herein and a pharmaceutically acceptable carrier or excipient.

According to some aspects, the disclosure provides for a lipid nanoparticle formulation further comprising one or more pharmaceutical excipients. According to some embodiments, the lipid nanoparticle formulation further comprises sucrose, tris, trehalose and/or glycine.

The ceDNA vector can be complexed with the lipid portion of the particle or encapsulated in the lipid position of the lipid nanoparticle. According to some embodiments, the ceDNA can be fully encapsulated in the lipid position of the lipid nanoparticle, thereby protecting it from degradation by a nuclease, e.g., in an aqueous solution. According to some embodiments, the ceDNA in the lipid nanoparticle is not substantially degraded after exposure of the lipid nanoparticle to a nuclease at 37° C. for at least about 20, 30, 45, or 60 minutes. According to some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. According to some aspects, the lipid nanoparticle formulation is a lyophilized powder.

According to some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

According to some further embodiments, the lipid nanoparticles having a non-lamellar morphology are electron dense. According to some aspects, the disclosure provides for a lipid nanoparticle that is either unilamellar or multilamellar in structure. According to some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size. The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (20 1 0), both of which are incorporated by reference in their entirety). The preferred range of pKa is ~5 to ~7. The pKa of the cationic/ionizable lipid can be determined in lipid nanoparticles using an assay based on fluorescence of 2-(p-toluidino)-6-napthalene sulfonic acid (TNS).

### VIII. Methods of Treatment

Methods are provided herein for inducing an immune response in a subject in need thereof comprising administering an immunologically effective amount of a vaccine regimen as disclosed herein. In some embodiments, methods are provided herein for inducing an immune response against a pathogenic organism in a subject in need thereof comprising administering an immunologically effective amount of a vaccine regimen as disclosed herein.

Some embodiments provide the use of the constructs or compositions disclosed herein for inducing an immune response to a first and a second peptide in a subject in need thereof. Some embodiments provide the use of the construct or composition as disclosed herein in a vaccine regimen. Some embodiments provide the use of the construct or composition as disclosed herein in the manufacture of a medicament inducing an immune response to an antigen in a subject.

Provided herein are methods of inducing an immune response against a first peptide and a second peptide in a subject, comprising administering a priming vaccine comprising a deoxyribonucleic acid (DNA) DNA to the subject, wherein the DNA encodes a first peptide; and administering a boosting vaccine comprising (i) a ribonucleic acid (RNA), or (ii) a second peptide to the subject, wherein the RNA encodes the second peptide, thereby inducing the immune response against the first peptide and the second peptide in the subject.

Vaccine regimens, comprising a priming vaccine comprising a deoxyribonucleic acid (DNA) DNA, wherein the DNA encodes a first peptide; and a boosting vaccine comprising (i) a ribonucleic acid (RNA), or (ii) a second peptide, wherein the RNA encodes the second peptide, are also provided.

Targets for the antibodies, or antigen-binding fragments described herein, (i.e., antigens) may be selected from a variety of pathogens, including, e.g., bacterial, viral, fungal and parasitic infectious agents. Suitable targets may further include cancer or cancer-associated antigens, or the like. Still other targets may include an autoimmune condition such as rheumatoid arthritis (RA) or multiple sclerosis (MS).

Targets for the immunoglobulin constructs described herein may be selected from a variety of pathogens, including, e.g., bacterial, viral, fungal and parasitic infectious agents. Suitable targets may further include cancer or cancer-associated antigens, or the like. Still other targets may include an autoimmune condition such as rheumatoid arthritis (RA) or multiple sclerosis (MS).

Examples of viral targets include influenza virus from the orthomyxovirudae family, which includes: Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7.

Broadly neutralizing antibodies against influenza A have been described. As used herein, a “broadly neutralizing antibody” refers to a neutralizing antibody which can neutralize multiple strains from multiple subtypes. For example, CR6261 [The Scripps Institute/Crucell] has been described as a monoclonal antibody that binds to a broad range of the influenza virus including the 1918 “Spanish flu” (SC1918/H1) and to a virus of the H5N1 class of avian influenza that jumped from chickens to a human in Vietnam in 2004 (Viet04/H5). CR6261 recognizes a highly conserved helical region in the membrane-proximal stem of hemagglutinin, the predominant protein on the surface of the influenza virus. This antibody is described in WO 2010/130636, incorporated by reference herein. Another neutralizing antibody, F10 [XOMA Ltd] has been described as being useful against HIN1 and H5N1. [Sui et al, Nature Structural and Molecular Biology (Sui, et al. 2009, 16(3):265-73)] Other antibodies against influenza, e.g., Fab28 and Fab49, may be selected. See, e.g., WO 2010/140114 and WO 2009/115972, which are incorporated by reference. Still other antibodies, such as those described in WO 2010/010466, US Published Patent Publication US/2011/076265, and WO 2008/156763, may be readily selected.

Other target pathogenic viruses include, arenaviruses (including funin, machupo, and Lassa), filoviruses (including Marburg and Ebola), hantaviruses, picornaviridae (including rhinoviruses, echovirus), coronaviruses, paramyxovirus, morbillivirus, respiratory syncytial virus, togavirus, coxsackievirus, parvovirus B19, parainfluenza, adenoviruses, reoviruses, variola (Variola major (Smallpox)) and Vaccinia (Cowpox) from the poxvirus family, and varicella-zoster (pseudorabies).

Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease.

A neutralizing antibody construct against a bacterial pathogen may also be selected for use in the present disclosure. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The causative agent of anthrax is a toxin produced by Bacillus anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Other infectious diseases may be caused by airborne fungi including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

In addition, passive immunization may be used to prevent fungal infections (e.g., athlete's foot), ringworm, or viruses, bacteria, parasites, fungi, and other pathogens which can be transmitted by direct contact. In addition, a variety of conditions which affect household pets, cattle and other livestock, and other animals. For example, in dogs, infection of the upper respiratory tract by canine sinonasal aspergillosis causes significant disease. In cats, upper respiratory disease or feline respiratory disease complex originating in the nose causes morbidity and mortality if left untreated. Cattle are prone to infections by the infectious bovine rhinotracheitis (commonly called IBR or red nose) is an acute, contagious virus disease of cattle. In addition, cattle are prone to Bovine Respiratory Syncytial Virus (BRSV) which causes mild to severe respiratory disease and can impair resistance to other diseases. Still other pathogens and diseases will be apparent to one of skill in the art. See, e.g., U.S. Pat. No. 5,811,524, which describes generation of anti-respiratory syncytial virus (RSV) neutralizing antibodies. The techniques described therein are applicable to other pathogens. Such an antibody may be used intact or its sequences (scaffold) modified to generate an artificial or recombinant neutralizing antibody construct. Such methods have been described [see, e.g., WO 2010/13036; WO 2009/115972; WO 2010/140114].

Anti-neoplastic immunoglobulins as described herein may target a human epidermal growth factor receptor (HER), such as HER2. For example, trastuzumab is a recombinant IgG1 kappa, humanized monoclonal antibody that selectively binds with high affinity in a cell-based assay (Kd=5 nM) to the extracellular domain of the human epidermal growth factor receptor protein. The commercially available product is produced in CHO cell culture. See, e.g., drugbank.ca/drugs/DB00072. The amino acid sequences of the trastuzumab light chains 1 and 2 and heavy chains 1 and 2, as well as sequences obtained from a study of the x-ray structure of trastuzumab, are provided on this database at accession number DB00072, which sequences are incorporated herein by reference. See, also, 212-Pb-TCMC-trastuzumab [Areva Med, Bethesda, Md.]. Another antibody of interest includes, e.g., pertuzumab, a recombinant humanized monoclonal antibody that targets the extracellular dimerization domain (Subdomain II) of the human epidermal growth factor receptor 2 protein (HER2). It consists of two heavy chains and two lights chains that have 448 and 214 residues respectively. FDA approved Jun. 8, 2012. The amino acid sequences of its heavy chain and light chain are provided, e.g., in www.drugbank.ca/drugs/DB06366 (synonyms include 2C4, MOAB 2C4, monoclonal antibody 2C4, and rhuMAb-2C4) on this database at accession number DB06366. In addition to HER2, other HER targets may be selected.

For example, MM-121/SAR256212 is a fully human monoclonal antibody that targets the HER3 receptor [Merrimack's Network Biology] and which has been reported to be useful in the treatment of non-small cell lung cancer (NSCLC), breast cancer and ovarian cancer. SAR256212 is an investigational fully human monoclonal antibody that targets the HER3 (ErbB3) receptor [Sanofi Oncology]. Another anti-Her3/EGFR antibody is RG7597 [Genentech], described as being useful in head and neck cancers. Another antibody, margetuximab (or MGAH22), a next-generation, Fc-optimized monoclonal antibody (mAb) that targets HER [MacroGenics], may also be utilized.

Alternatively, other human epithelial cell surface markers and/or other tumor receptors or antigens may be targeted. Examples of other cell surface marker targets include, e.g., 5T4, CA-125, CEA (e.g., targeted by labetuzumab), CD3, CD19, CD20 (e.g., targeted by rituximab), CD22 (e.g., targeted by epratuzumab or veltuzumab), CD30, CD33, CD40, CD44, CD51 (also integrin αvβ3), CD133 (e.g., glioblastoma cells), CTLA-4 (e.g., Ipilimumab used in treatment of, e.g., neuroblastoma)), Chemokine (C—X-C Motif) Receptor 2 (CXCR2) (expressed in different regions in brain; e.g., Anti-CXCR2 (extracellular) antibody #ACR-012 (Alomene Labs)); EpCAM, fibroblast activation protein (FAP) [see, e.g., WO 2012020006 A2, brain cancers], folate receptor alpha (e.g., pediatric ependymal brain tumors, head and neck cancers), fibroblast growth factor receptor 1 (FGFR1) (see, et al, WO2012125124A1 for discussion treatment of cancers with anti-FGFR1 antibodies), FGFR2 (see, e.g., antibodies described in WO2013076186A and WO2011143318A2), FGFR3 (see, e.g., antibodies described in U.S. Pat. No. 8,187,601 and WO2010111367A1), FGFR4 (see, e.g., anti-FGFR4 antibodies described in WO2012138975A1), hepatocyte growth factor (HGF) (see, e.g., antibodies in WO2010119991A3), integrin α5β1, IGF-1 receptor, gangioloside GD2 (see, e.g., antibodies described in WO2011160119A2), ganglioside GD3, transmembrane glycoprotein NMB (GPNMB) (associated with gliomas, among others and target of the antibody glembatumumab (CR011), mucin, MUC1, phosphatidylserine (e.g., targeted by bavituximab, Peregrine Pharmaceuticals, Inc], prostatic carcinoma cells, PD-L1 (e.g., nivolumab (BMS-936558, MDX-1106, ONO-4538), a fully human gG4, e.g., metastatic melanoma], platelet-derived growth factor receptor, alpha (PDGFR a) or CD140, tumor associated glycoprotein 72 (TAG-72), tenascin C, tumor necrosis factor (TNF) receptor (TRAIL-R2), vascular endothelial growth factor (VEGF)-A (e.g., targeted by bevacizumab) and VEGFR2 (e.g., targeted by ramucirumab).

Other antibodies and their targets include, e.g., APN301 (hu14.19-IL2), a monoclonal antibody [malignant melanoma and neuroblastoma in children, Apeiron Biolgics, Vienna, Austria]. See, also, e.g., monoclonal antibody, 8H9, which has been described as being useful for the treatment of solid tumors, including metastatic brain cancer. The monoclonal antibody 8H9 is a mouse IgG1 antibody with specificity for the B7H3 antigen [United Therapeutics Corporation]. This mouse antibody can be humanized Still other immunoglobulin constructs targeting the B7-H3 and/or the B7-H4 antigen may be used herein. Another antibody is S58 (anti-GD2, neuroblastoma). COTARA [Perregrince Pharmaceuticals] is a monoclonal antibody described for treatment of recurrent glioblastoma. Other antibodies may include, e.g., avastin, ficlatuzumab, medi-575, and olaratumab. Still other immunoglobulin constructs or monoclonal antibodies may be selected for use herein. See, e.g., Medicines in Development Biologics, 2013 Report, pp. 1-87, a publication of PhRMA's Communications & Public Affairs Department. (202) 835-3460, which is incorporated by reference herein.

For example, immunogens may be selected from a variety of viral families. Example of viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies).

Within the rhabdovirus family, suitable antigens may be derived from the G protein or the N protein. The family filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bunyaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected as targets. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. See, U.S. Pat. Nos. 5,891,994 and 6,193,981. See, also, the HIV and SIV proteins described in D. H. Barouch et al, J. Virol., 75(5):2462-2467 (March 2001), and R. R. Amara, et al, Science, 292:69-74 (6 Apr. 2001). In another example, the HIV and/or SIV immunogenic proteins or peptides may be used to form fusion proteins or other immunogenic molecules. See, e.g., the HIV-1 Tat and/or Nef fusion proteins and immunization regimens described in WO 01/54719, published Aug. 2, 2001, and WO 99/16884, published Apr. 8, 1999. The invention is not limited to the HIV and/or SIV immunogenic proteins or peptides described herein. In addition, a variety of modifications to these proteins has been described or could readily be made by one of skill in the art. See, e.g., the modified gag protein that is described in U.S. Pat. No. 5,972,596.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family includes feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

Other pathogenic targets for antibodies may include, e.g., bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include Listeria monocytogenes; Erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoa and helminthes and infections produced thereby include: amebiasis; malaria; leishmaniasis; trypanosomiasis; toxoplasmosis; Pneumocystis cannii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to target antigens from these organisms, viruses, their toxins or other by-products, which will prevent and/or treat infection or other adverse reactions with these biological agents.

The subject that is administered the ceDNA vector may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g., subject resides in a densely-populated city or in close proximity to subjects having confirmed or suspected infections of a virus, or choice of employment, e.g., hospital worker, pharmaceutical researcher, traveler to infected area, or frequent flier.

The present disclosure also encompasses prophylactically administering a ceDNA vector for expression of antigen, or immunogenic peptide, as described herein, to a subject who is at risk of a disease or disorder, e.g., av iral infection so as to prevent such infection. “Prevent” or “preventing” means to administer a ceDNA vector for expression of antigen, or immunogenic peptide, as described herein, to a subject to inhibit the manifestation of a disease or infection (e.g., viral infection) in the body of a subject, for which the ceDNA vector for expression of peptides (e.g., antigens) as described herein is effective when administered to the subject at an effective or therapeutically effective amount or dose.

According to some embodiments, a sign or symptom of a viral infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection are discussed herein.

As noted above, according to some embodiments the subject may be a non-human animal, and the antibodies and antigen-binding fragments discussed herein may be used in a veterinary context to treat and/or prevent disease in the non-human animals (e.g., cats, dogs, pigs, cows, horses, goats, rabbits, sheep, and the like).

The present disclosure provides a method for treating or preventing viral infection (e.g., coronavirus infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of a vaccine regimen as described herein to the subject.

### ELISpot Assay to Detect Cytokine-Secreting Cells

The filter immunoplaque assay, otherwise called the enzyme-linked immunospot assay (ELISpot), was initially developed to detect and quantitate individual antibody-secreting B cells. The technique originally provided a rapid and versatile alternative to conventional plaque-forming cell assays. Recent modifications have improved the sensitivity of the ELISpot assay such that cells producing as few as 100 molecules of specific protein per second can be detected. These assays take advantage of the relatively high concentration of a given protein (such as a cytokine) in the environment immediately surrounding the protein-secreting cell. These cell products are captured and detected using high-affinity antibodies.

The ELISpot assay utilizes two high-affinity cytokine-specific antibodies directed against different epitopes on the same cytokine molecule: either two monoclonal antibodies or a combination of one monoclonal antibody and one polyvalent antiserum. ELISpot generates spots based on a colorimetric reaction that detects the cytokine secreted by a single cell. The spot represents a “footprint” of the original cytokine-producing cell. Spots (i.e., spot forming cells or SFC) are permanent and can be quantitated visually, microscopically, or electronically.

According to some embodiments, the performance of the ELISpot assay to the present disclosure measures the number of CD8⁺ T cells (CTLs) and CD4⁺ T cells induced in response to the prime/boost vaccine regimen disclosed herein, as measured by the production of gamma interferon.

### Detection of Cell-Mediated Immune Responses

Cell-mediated immune responses to tested antigens can be analyzed using several of the most appropriate cell based assays, which include the 51Cr-release CTL assay (Coligan J, Kruisbeek A, Margulies D, Shevach E, Strober W, eds. Current protocols in immunology. New York: Wiley Interscience), soluble MHC Class I tetramer staining, ELISpot assay, and intracellular cytokine analysis.

### Ex Vivo Treatment

According to some embodiments, cells are removed from a subject, a ceDNA vector for expression of peptides (e.g., antigens) as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of peptides (e.g., antigens) as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

According to some embodiments, a ceDNA vector for expression of peptides (e.g., antigens) as disclosed herein can encode an antibody, and antigen-binding fragment thereof, as described herein that is to be produced in a cell in vitro, ex vivo, or in vivo. For example, in contrast to the use of the ceDNA vectors described herein in a method of treatment as discussed herein, according to some embodiments a ceDNA vector for expression of peptides (e.g., antigens) may be introduced into cultured cells and the expressed peptides (e.g., antigens) isolated from the cells after a period of time, e.g., for the production of antibodies and fusion proteins. According to some embodiments, the cultured cells comprising a ceDNA vector for expression of peptides (e.g., antigens) as disclosed herein can be used for commercial production of antibodies or fusion proteins, e.g., serving as a cell source for small or large scale biomanufacturing of antibodies or fusion proteins. In alternative embodiments, a ceDNA vector for expression of peptides (e.g., antigens) as disclosed herein is introduced into cells in a host non-human subject, for in vivo production of antibodies or fusion proteins, including small scale production as well as for commercial large scale peptides (e.g., antigens) production.

The ceDNA vectors for expression of antigens and immunogenic peptides as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### Dose Ranges

Provided herein are methods of treatment comprising administering to the subject an effective amount of a composition comprising a ceDNA vector encoding peptides (e.g., antigens) as described herein.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems

A ceDNA vector for expression of peptides (e.g., antigens) as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vectors for expression of peptides (e.g., antigens) as disclosed herein required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

An effective or therapeutically effective dose of a ceDNA vector for expression of antigens and immunogenic peptides as described herein, for treating or preventing a viral infection refers to the amount of the ceDNA vector for expression of antigen, or immunogenic peptide, as described herein, antigens and immunogenic peptides that is sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the disclosure, an effective or therapeutically effective dose of antibody or antigen-binding fragment thereof of the present disclosure, for treating or preventing viral infection, e.g., in an adult human subject, is about 0.01 to about 200 mg/kg, e.g., up to about 150 mg/kg. In an embodiment of the disclosure, the dosage is up to about 10.8 or 11 grams (e.g., about 1, 2, 3, 4, 5, 6, 7, 8, 9, 10 or 11 grams).

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” for clinical use will fall in a relatively broad range that can be determined through clinical trials and will depend on the particular application (e.g., neural cells will require very small amounts, while systemic injection would require large amounts). For example, for direct in vivo injection into skeletal or cardiac muscle of a human subject, a therapeutically effective dose will be on the order of from about 1 μg to 100 μg of the ceDNA vector. If exosomes or microparticles are used to deliver the ceDNA vector, then a therapeutically effective dose can be determined experimentally, but is expected to deliver from 1 μg to about 100 μg of vector. Moreover, a therapeutically effective dose is an amount ceDNA vector that expresses a sufficient amount of the transgene to have an effect on the subject that results in a reduction According to some or more symptoms of the disease, but does not result in significant off-target or significant adverse side effects. According to some embodiments, a “therapeutically effective amount” is an amount of an expressed peptides (e.g., antigens) that is sufficient to produce a statistically significant, measurable change in reduction of a given disease symptom. Such effective amounts can be gauged in clinical trials as well as animal studies for a given ceDNA vector composition.

For in vitro transfection, an effective amount of a ceDNA vectors for expression of peptides (e.g., antigens) as disclosed herein to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. According to some embodiments, more than one dose can be administered to a subject; in fact, multiple doses can be administered as needed, because the ceDNA vector does not elicit an anti-capsid host immune response due to the absence of a viral capsid. As such, one of skill in the art can readily determine an appropriate number of doses. According to some embodiments, the doses are administered in a prime-dose dosing regime.

Without wishing to be bound by any particular theory, the lack of typical anti-viral immune response elicited by administration of a ceDNA vector as described by the disclosure (i.e., the absence of capsid components) allows the ceDNA vector for expression of peptides (e.g., antigens) to be administered to a host on multiple occasions. According to some embodiments, the number of occasions in which a nucleic acid is delivered to a subject is in a range of 2 to 10 times (e.g., 2, 3, 4, 5, 6, 7, 8, 9, or 10 times). According to some embodiments, a ceDNA vector is delivered to a subject more than 5 times. According to some embodiments, a ceDNA vector is delivered to a subject more than 3 times. According to some embodiments, a ceDNA vector is delivered to a subject more than 2 times.

### Unit Dosage Forms

According to some embodiments, the pharmaceutical compositions comprising a prime vaccine composition for expression of peptides (e.g., antigens) or comprising a boost vaccine composition for expression of peptides (e.g., antigens) as disclosed herein can conveniently be presented in unit dosage form. A unit dosage form will typically be adapted to one or more specific routes of administration of the pharmaceutical composition.

According to some embodiments, the unit dosage form is adapted for intravenous, intramuscular, or subcutaneous administration. According to some embodiments, the unit dosage form is adapted for administration by inhalation. According to some embodiments, the unit dosage form is adapted for administration by a vaporizer. According to some embodiments, the unit dosage form is adapted for administration by a nebulizer. According to some embodiments, the unit dosage form is adapted for administration by an aerosolizer. According to some embodiments, the unit dosage form is adapted for oral administration, for buccal administration, or for sublingual administration.

### Methods for Producing Molecules of Interest

The compositions and methods of the present disclosure can be used to produce (e.g., express) a molecule of interest such as, e.g., a polypeptide, encoded in an open reading frame of a gene of interest (GOI) as disclosed herein. Thus, the present application further provides compositions and methods for producing a molecule of interest such as, e.g., a polypeptide.

Accordingly, some embodiments relate to methods for producing a polypeptide of interest in a subject, including administering to the subject the prime and the boost vaccine compositions described herein according to any one of the aspects and embodiments.

The methods and compositions disclosed herein can be used, for example, with subjects including those that are used in aquaculture, agriculture, animal husbandry, and/or for therapeutic and medicinal applications, including production of polypeptides used in the manufacturing of vaccines, pharmaceutical products, industrial products, chemicals, and the like. In some embodiments, the compositions and methods disclosed herein can be used with subjects that are natural hosts of alphaviruses, such as rodents, mice, fish, birds, and larger mammals such as humans, horses, pig, monkey, and apes as well as invertebrates. In some embodiments, subjects are vertebrate animal species and invertebrate animal species. Any animal species can be generally used and can be, for example, mammalian species such as human, horse, pig, primate, mouse, ferret, rat, cotton rat, cattle, swine, sheep, rabbit, cat, dog, goat, donkey, hamster, or buffalo. In some embodiments, the subject is an avian species, a crustacean species, or a fish species.

Techniques for transforming or transfecting a wide variety of the above-mentioned subjects are known in the art and described in the technical and scientific literature.

### IX. Kits

The invention provides a pharmaceutical kit for the ready administration of an immunogenic, prophylactic or therapeutic regimen for treating a disease or condition, e.g., one caused by a pathogenic organism. The kit is designed for use in a method for inducing an immune response in a subject, the method comprising administering to the subject at least one dose of a priming composition comprising a ceDNA vector which encodes a first immunogenic peptide or antigen; and subsequently administering to the subject at least one dose of a boosting composition, wherein the boosting composition comprises an mRNA which encodes a second immunogenic peptide or antigen.

The kit contains at least one immunogenic composition comprising an ceDNA encoding an antigen and at least one immunogenic composition comprising an RNA molecule encoding an antigen. The kit contains at least one immunogenic composition comprising an ceDNA encoding an antigen and at least one immunogenic composition comprising an amino acid sequence encoding an antigen. The kit may contain multiple prepackaged doses of each of the component vectors for multiple administrations of each. Components of the kit may be contained in vials.

The invention provides a pharmaceutical kit for the ready administration of an immunogenic, prophylactic or therapeutic regimen for treating a disease or condition caused by an infectious pathogenic organism. The kit is designed for use in any of the methods described herein.

The kit contains at least one immunogenic composition comprising a ceDNA vector encoding an antigen and at least one immunogenic composition comprising an RNA molecule encoding an antigen. The kit may contain multiple prepackaged doses of each of the component vectors for multiple administrations of each. Components of the kit may be contained in vials.

The kit also contains instructions for using the immunogenic compositions in the prime/boost methods described herein. It may also contain instructions for performing assays relevant to the immunogenicity of the components. The kit may also contain excipients, diluents, adjuvants, syringes, other appropriate means of administering the immunogenic compositions or decontamination or other disposal instructions.

Vectors of the invention are generated using techniques and sequences provided herein, in conjunction with techniques known to those of skill in the art. Such techniques include conventional cloning techniques of cDNA such as those described in texts, use of overlapping oligonucleotide sequences of the adenovirus genomes, polymerase chain reaction, and any suitable method which provides the desired nucleotide sequence.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of the ceDNA vectors using a polynucleotide construct template is described in Example 1 of PCT/US18/49996, which is incorporated herein in its entirety by reference. For example, a polynucleotide construct template used for generating the ceDNA vectors of the present disclosure can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

### Example 2: Synthetic ceDNA Production Via Excision from a Double-Stranded DNA Molecule

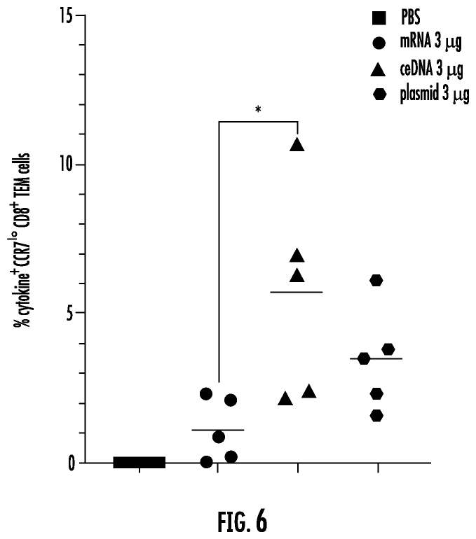

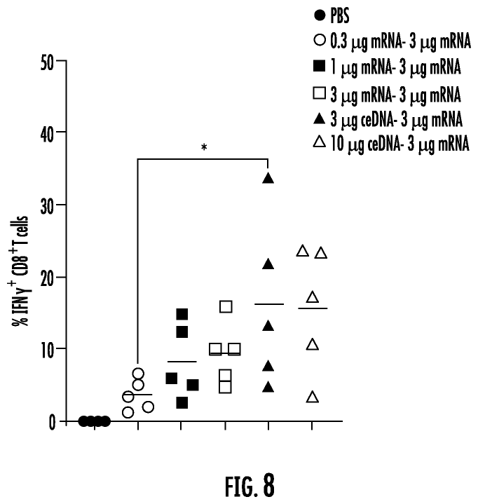

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. According to some embodiments, the double stranded DNA construct is a ceDNA plasmid, e.g., see, e.g., FIG. 6 in International patent application PCT/US2018/064242, filed Dec. 6, 2018).

According to some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see, e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about −54.4 kcal/mol. Other modifications to the ITR may also be made, including optional deletion of a functional Rep binding site or a Trs site.

### Example 3: CeDNA Production Via Oligonucleotide Construction

Another exemplary method of producing a ceDNA vector using a synthetic method that involves assembly of various oligonucleotides, is provided in Example 3 of PCT/US19/14122, incorporated by reference in its entirety herein, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

As disclosed herein, the ITR oligonucleotides can comprise WT-ITRs, or modified ITRs. (See, e.g., FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 2 and 3 can comprise WT-ITRs, or modified ITRs (mod-ITRs) in symmetrical or asymmetrical configurations, as discussed herein.

### Example 4: CeDNA Production Via a Single-Stranded DNA Molecule

Another exemplary method of producing a ceDNA vector using a synthetic method is provided in Example 4 of PCT/US19/14122, incorporated by reference in its entirety herein, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′:
a sense first ITR;
a sense expression cassette sequence;
a sense second ITR;
an antisense second ITR;
an antisense expression cassette sequence; and
an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: A Study to Evaluate Anti-Spike Antibody Responses after Intramuscular Administration of LNP:DNA or LNP:mRNA Formulations in Female BALB/c Mice

The objective of the study was to evaluate the anti-spike protein antibody response after intramuscular (IM) injection of LNP:DNA or LNP:mRNA formulations as in a prime-boost regimen. The study design and details were carried out as set forth below.

### Study Design

Table 13 sets forth the design of the study. SARS-CoV-2 prefusion stabilized full length Spike protein antigen was delivered by IM injection as either LNP:ceDNA or LNP:mRNA C9 ionizable lipid-based formulations. Boost was either with same agent as the priming dose at day 0, or was mixed (e.g., ceDNA (prime)-ceDNA (boost), ceDNA (prime)-mRNA(boost), mRNA(prime)-mRNA(boost), or mRNA(prime)-ceDNA(boost)).

TABLE 13

| Group | | | | |
|---|---|---|---|---|
| (female | Day 0 TA | | Boost TA | |
| Balb/C | (3 μg IM | Boost | (3 μg IM | |
| n = 5) | in 30 μl) | Day | in 30 μl) | Endpoints |
| 1 | PBS | 28 | PBS | Cytokines 6 h |
| 2 | ceDNA | | ceDNA | after each dose |
| 3 | ceDNA | | mRNA | Serum |
| 4 | mRNA | | mRNA | Day 21, 49 |
| 5 | mRNA | | ceDNA | Splenocytes |
| | | | | Day 49 |
| 6 | PBS | 56 | PBS | Cytokines 6 h |
| 7 | ceDNA | | ceDNA | after each dose |
| 8 | ceDNA | | ceDNA | Serum |
| 9 | mRNA | | mRNA | Day 21, 49, 77 |
| 10 | mRNA | | mRNA | Splenocytes |
| | | | | Day 77 |
| 11 | PBS | 84 | PBS | Cytokines 6 h |
| 12 | ceDNA | | ceDNA | after each dose |
| 13 | ceDNA | | ceDNA | Serum |
| 14 | mRNA | | mRNA | Day 21, 49, 77, 105 |
| 15 | mRNA | | mRNA | Splenocytes |
| | | | | Day 105 |

### Test System

The test system was as follows:
Species: Mus musculus
Strain: Balb/c mouse
Number of females: 75, plus 3 spares
Age: 6 weeks of age at arrival
Source: Charles River Laboratories

Housing: Animals were group housed in clear polycarbonate cages with contact bedding in a procedure room.

Food and Water: Animals were provided ad libitum Mouse Diet 5058 and filtered tap water acidified with 1N HCl to a targeted pH of 2.5-3.0.

### Test Material

Class of Compound: Recombinant DNA Vector: ceDNA & mRNA.

Dose Formulation: Test articles were supplied in a concentrated stock. Test article concentration was recorded at time of receipt.

Stock was warmed to room temperature and diluted with the provided PBS immediately, as necessary, prior to use. Prepared materials were stored at −4° C. if dosing was not performed immediately.

Test Material Administration: Test (or control) Material #1 was dosed at 30 μL per animal on Day 28 for all Groups 1-15.

Test (or control) Material #2 was dosed at 30 μL per animal on Day 28 for Groups 1-5; Day 56 for Groups 6-10 and Day 84 for Groups 11-15.

Animals were dosed by intramuscular administration into the LEFT gastrocnemius. Animals were anesthetized with inhalant isoflurane, to effect, for dose procedures.

Residual Materials: All residual open stock was retained for future dosing, refrigerated. Diluted dose materials were discarded after the completion of the dose administration.

### In-Life Observations and Measurements

Cage Side Observations (Animal Health Checks): Cage side animal health checks were performed at least once daily to check for general health, mortality and moribundity.

Clinical Observations: Clinical observations were performed on Days 0 & 28, 56 or 84: 60-120 minutes post each dose and at the end of the work day (3-6 hours post) and on Days 1 & 29: 22-26 hours post the Day 0 & 28 Test Material dose. Only those animals that receive test material administration on Days 28, 56 or 84 required clinical observations post dose. Special attention was paid to the left hind limb, as the site of injection.

Body Weights: Body weights for all animals (as applicable for remaining animals), will be recorded on Days 0, 1, 2, 3, 7, 14, 21, 28, 29, 30, 31, 35, 42, 49, 56, 57, 58, 59, 57, 58, 59, 63, 70, 77, 84, 85, 86, 87, 91, 98 and 105. Additional body weights were recorded as requested.

Anasthesia and Recovery: Animals were monitored continuously while under anesthesia, during recovery and until mobile, per testing facility SOPs.

### Blood Collection

Animals in Groups 1-15, hade interim blood for serum collected on dose Days 0 & 28, 56 or 77; 4-6 hours post Test Material dose as shown in Table 14 below. Animals in Groups 1-5 had interim blood for serum collected on Day 21. Animals in Groups 6-10 had interim blood for serum collected on Days 21 & 49. Animals in Groups 11-15 had interim blood for serum collected on Days 21, 49 & 77.

All animals had whole blood for serum collection.

Whole blood for serum was collected by orbital or tail collection. Whole blood was collected into a serum separator with clot activator tube and processed into one (1) aliquot of serum per facility SOPs.

All samples were stored at nominally −70° C. until shipped to on dry ice.

TABLE 14
Blood Collection (Interim; for cytokines)

| | Sample Collection Times |
|---|---|
| | Whole Blood |
| Group | (Tail, saphenous or orbital) |
| Number | Serum^{a} |
| 1-5 | Days 0 & 28 |
| | 4-6 hours post Test Material dose (±5%) |
| 6-10 | Days 0 & 56 |
| | 4-6 hours post Test Material dose (±5%) |
| 11-15 | Days 0 & 84 |
| | 4-6 hours post Test Material dose (±5%) |
| Volume / | ~150 μL whole blood |
| Portion | |
| Processing / | 1 aliquot frozen at nominally −70° C. |
| Storage | (Charles River) |

^{a}Whole blood was collected into serum separator tubes, with clot activator

TABLE 15
Blood Collection (Interim)

| | Whole Blood Sample Collection Times |
|---|---|
| | Whole Blood |
| Group | (Tail, saphenous or orbital) |
| Number | Serum^{a} |
| 1-5 | Day 21 |
| 6-10 | Days 21 & 49 |
| 11-15 | Days 21, 49 & 77 |
| Volume / Portion | ~100 μL whole blood |
| Processing | One (1) aliquot |
| Storage | Frozen at nominally −70° C. |
| | (Gen Bio) |

^{a}Whole blood was collected into serum separator tubes, with clot activator

After each collection animals received 0.5-1.0 mL lactated Ringer's; subcutaneously.

Whole blood for serum was collected by saphenous vein. Whole blood was collected into a serum separator with clot activator tube and processed into one aliquot of serum.

All samples were stored at nominally −70° C. until shipped to on dry ice.

Anesthesia Recovery: As applicable, animals were monitored continuously while under anesthesia, during recovery and until mobile.

### Terminal Procedures and Collections

TABLE 16
Blood Collection- Terminal

| | Sample Collection Times |
|---|---|
| | Terminal Whole Blood |
| Group | (cardiac) |
| Number | Serum^{a} |
| 1-5 | Day 49 |
| 6-10 | Day 77 |
| 11-15 | Day 105 |
| Portion | MOV |
| Processing / | Four (4) aliquots frozen |
| Storage | at nominally −70° C. |

MOV = maximum obtainable volume
^{a}Whole blood was collected into serum separator tubes, with clot activator

TABLE 17
Tissue Collection- Terminal

| | Sample Collection Times |
|---|---|
| Group | Spleen |
| Number | Processed for Splenocytes |
| 1-5 | Day 49 |
| 6-10 | Day 77 |
| 11-15 | Day 105 |
| Portion | Whole Organ |
| Processing / | Processed by Testing Facility and |
| Storage | Frozen in Freezing Medium |

Terminal Blood: For all animals, whole blood from exsanguination was collected into a serum separator with clot activator tube and processed into four (4) aliquots of serum per facility SOPs.

All samples were stored at nominally −70° C. until shipped to on dry ice.

Terminal Tissues: For Groups 1-15, spleens were harvested and weighed. Spleens were processed into splenocytes using Miltenyi Dissociation Kit per Testing Facility Protocol. After processing, spleens were counted, pelleted, and resuspended at 10 million cells per mL. Yield and dissociated cell viability was recorded. Up to 60 million cells (<6 aliquots) were frozen as a suspension in Cell Culture Freezing medium (Gibco #12648010), at 10 million cells per mL. Any additional cells were discarded.

Cells were stored at nominally −70° C. until shipped on dry ice.

Euthanasia: Animals were euthanized on Day 49, 77 or 105. Animals were euthanized by CO2 asphyxiation followed by thoracotomy and exsanguination.

### Results

The foregoing Example describes immunogenic prime boost regimens using the prefusion stabilized full length SARS-CoV-2 Spike protein as a model antigen to characterize the immune response elicited by the LNP:ceDNA or LNP:mRNA test formulations. This antigen was chosen as a proof of principle to demonstrate the universality of prime boost combinations comprising LNP:ceDNA as the priming agent.

As described above, animals were immunized at day 0 and boosted on either Day 28, which is the standard SpikeVax schedule, Day 56 or Day 84. The booster dose was with the same agent as the priming dose, or was different, e.g., ceDNA (prime)-ceDNA (boost), ceDNA (prime)-mRNA(boost), mRNA(prime)-mRNA(boost), or mRNA(prime)-ceDNA(boost).

FIG. 1 shows spike protein antibody titer as determined on Day 49 of the study. The mRNA construct was used as the benchmark for comparison of spike protein antibody titer. As shown in FIG. 1, on day 49 post-treatment, one dose of ceDNA produced similar spike binding titers as one dose of mRNA. FIG. 2 shows spike protein antibody titer as determined on Day 77 of the study. The mRNA construct was used as the benchmark for comparison of spike protein antibody titer. As shown in FIG. 2, on day 77 post-treatment (day 0 prime, day 56 boost), one dose of ceDNA produced similar spike binding titers as one dose of mRNA. FIG. 3 shows spike protein antibody titer as determined on Day 105 of the study. The mRNA construct was used as the benchmark for comparison of spike protein antibody titer. As shown in FIG. 3, on day 105 post-treatment (day 0 prime, day 56 boost; day 0 prime, day 84 boost), one dose of ceDNA produced similar spike binding titers as one dose of mRNA. The results in FIGS. 1-3 showed that the heterologous prime-boost strategy using ceDNA as a prime could efficiently induce the production of higher titers of antibodies in mice.

FIG. 4 is a graph that depicts the percentage of CD8⁺ T cells in the population that were IFNγ⁺, IFNγ⁺ and CD107⁺, IFNγ⁺ and TNFα⁺ or IL4⁺ at assay day 77 (day 0 prime, day 56 boost).

FIG. 4 demonstrates that the ceDNA prime-mRNA boost dose regimen induced significantly more CD8⁺ T cells that produce IFNγ or IFNγ and TNFα or IFNγ, and that display cytolytic functions (using CD107⁺ as a marker for cytolytic degranulation) in response to S1 peptide pool, than the homologous prime-boost strategies or the heterologous prime-boost strategies that do not employ ceDNA as the prime and mRNA as the boost. One rationale for this result may be that that mixing the prime boost as well as the 8 week spacing are playing a role in this response. Expression of the cytokine IL4 is a hallmark of the allergic response and is inappropriate in the viral response. As shown in FIG. 4, IL4 levels were low.

In summary, the data in Example 5 show that ceDNA vaccine platforms can be successfully combined in heterologous prime/boost regimens for eliciting and enhancing both immune response to an encoded model antigen, and are important candidates for the design of a improved vaccine strategies.

### Example 6: A Study to Evaluate Anti-Spike Antibody Responses after Intramuscular Administration of LNP:DNA Formulations in Female BALB/c Mice

ceDNA vectors were produced according to the methods described in Example 1 above.

The objective of the study was to evaluate the anti-spike protein antibody response after intramuscular (IM) injection of LNP:ceDNA formulations. The study design and details were carried out as set forth below.

### Study Design

Table 18 sets forth the design of the study. As shown in Table 18, a ceDNA comprising a nucleic acid encoding SARS-CoV-2 spike protein antigen was dosed in 6 groups (Groups 2-7) of mice (n=5) at a dose level of 3 μg in a dose volume of 30 μl/animal. Group 1 served as the control. Dosing was performed on Day 0 and Day 28 by intramuscular (IM) injection. Day 49 was the terminal time point of the study.

TABLE 18

| | | | Dose | Dose | Dosing | Terminal |
|---|---|---|---|---|---|---|
| Group | No. of | | Levels | Volume | Regimen | Time |
| No. | Animals | Test Material | (μg) | (μL/an) | ROA | Point |
| 1 | 5 | PBS | NA | 30 | Once on | Day 49 |
| 2 | 5 | LNP 1: ceDNA | 3 | | Days 0 & 28 | |
| 3 | 5 | LNP 2: ceDNA | 3 | | by IM | |
| 4 | 5 | LNP 3: ceDNA | 3 | | | |
| 5 | 5 | LNP 4: ceDNA | 3 | | | |
| 6 | 5 | LNP 5: ceDNA | 3 | | | |
| 7 | 5 | LNP 6: ceDNA | 3 | | | |

No. = Number;
an = animal;
IM = intramuscular;
ROA = route of administration
LNP 1 = Ionizable lipid (C9):DSPC:Chol:DMG-PEG2000
LNP 2 = Ionizable lipid (C7):DSPC:Chol:DMG-PEG2000
LNP 3 = Ionizable lipid (C7):DOPE:Chol:DMG-PEG2000
LNP 4 = Ionizable lipid (C7):DSPC:Chol:DMG-PEG2000-COOH
LNP 5 = Ionizable lipid (C7):DSPC:Chol:DMG-PEG2000 1%
LNP 6 = Ionizable lipid (C7):DSPC:Chol:DMG-PEG2000 2.5%

### Test System

The test system was as follows:
Species: Mus musculus
Strain: Balb/c mouse
Number of females: 35, plus 3 spares
Age: 6 weeks of age at arrival
Source: Charles River Laboratories

Housing: Animals were group housed in clear polycarbonate cages with contact bedding in a procedure room.

Food and Water: Animals were provided ad libitum Mouse Diet 5058 and filtered tap water acidified with 1N HCl to a targeted pH of 2.5-3.0.

### Test Material

Class of Compound: Recombinant DNA Vector: ceDNA

Dose Formulation: Test articles were supplied in a concentrated stock. Test article concentration was recorded at time of receipt.

Stock was warmed to room temperature and diluted with the provided PBS immediately, as necessary, prior to use. Prepared materials were stored at ~4° C. if dosing was not performed immediately.

Test Material Administration: Test and control articles were dosed at 30 μL per animal on Days 0 and 28 for all Groups 1-7. Dosing was performed by intramuscular administration into the LEFT gastrocnemius. Animals were anesthetized with inhalant isoflurane, to effect, per facility SOPS for dose procedures.

Residual Materials: All residual open stock was retained for future dosing, refrigerated. Diluted dose materials were discarded after the completion of the dose administration.

### In-Life Observations and Measurements

Cage Side Observations (Animal Health Checks): Cage side animal health checks were performed at least once daily to check for general health, mortality and moribundity.

Clinical Observations: Clinical observations were performed on Days 0 & 28: 60-120 minutes post each dose and at the end of the work day (3-6 hours post) and on Days 1 & 29: 22-26 hours post the Day 0 & 28 Test Material dose.

Body Weights: Body weights for all animals (as applicable for remaining animals), were recorded on Days 0, 1, 2, 3, 7, 14, 21, 28, 29, 30, 31, 35, 42 and 49. Additional body weights were recorded as requested.

Anasthesia and Recovery: Animals were monitored continuously while under anesthesia, during recovery and until mobile, per testing facility SOPs.

### Blood Collection

All animals in Groups 1-7, had interim blood for serum collected on Day 0; 4-6 hours post Test Material dose as shown in Table 19 and Table 20 below.

Animals in Groups 1-8 had interim blood for serum collected on Day 21.

Whole blood for serum was collected by saphenous vein. Whole blood was collected into a serum separator with clot activator tube and processed into one (1) aliquot of serum per facility SOPs.

All samples were stored at nominally −70° C. until shipped to on dry ice.

TABLE 19
Blood Collection (Interim) for Cytokines

| | Sample Collection Times |
|---|---|
| | Whole Blood |
| Group | (Tail, saphenous or orbital) |
| Number | Serum^{a} |
| 1-7 | Day 0 |
| | 4-6 hours post Test Material dose (±5%) |
| Volume / | ~150 μL whole blood |
| Portion | |
| Processing / | 1 aliquot frozen at nominally −70° C. |
| Storage | (Charles River) |

^{a}Whole blood was collected into serum separator tubes, with clot activator

TABLE 20
Blood Collection (Interim)

| | Whole Blood Sample Collection Times |
|---|---|
| | Whole Blood |
| Group | (Tail, saphenous or orbital) |
| Number | Serum^{a} |
| 1-7 | Day 21 |
| Volume / | ~100 μL whole blood |
| Portion | |
| Processing | One (1) aliquot |
| Storage | Frozen at nominally −70° C. |
| | (Gen Bio) |

^{a}Whole blood was be collected into serum separator tubes, with clot activator

After each collection animals received 0.5-1.0 mL lactated Ringer's; subcutaneously.

Whole blood for serum was collected by saphenous vein. Whole blood was collected into a serum separator with clot activator tube and processed into one (1) aliquot (Days 0 & 28) or two (2) aliquots (Day 21) of serum. All samples were stored at nominally −70° C. until shipped to on dry ice.

Anesthesia Recovery: As applicable, animals were monitored continuously while under anesthesia, during recovery and until mobile.

### Tissue Collections

TABLE 21
Blood Collection- Terminal

| | Sample Collection Times |
|---|---|
| | Terminal Whole Blood |
| Group | (cardiac) |
| Number | Serum^{a} |
| 1-7 | Day 49 |
| Portion | MOV |
| Processing / | Four (4) aliquots frozen |
| Storage | at nominally −70° C. |

MOV = maximum obtainable volume
^{a}Whole blood was collected into serum separator tubes, with clot activator

TABLE 22
Tissue Collection- Terminal

| | Sample Collection Times |
|---|---|
| Group | Spleen |
| Number | Splenocytes |
| 1-7 | Day 49 |
| Portion | MOV |
| Processing / | Processed by Testing Facility and |
| Storage | Frozen in Freezing Medium |

For all animals, whole blood from exsanguination was collected into a serum separator with clot activator tube and processed into four (4) aliquots of serum per facility SOPs. All samples were stored at nominally −70° C. until shipped to on dry ice.

Terminal Tissues: For Groups 1-7, spleens were harvested and weighed. Spleens were processed into splenocytes using Miltenyi Dissociation Kit per Testing Facility Protocol. After processing, spleens were counted, pelleted, and resuspended. Yield and dissociated cell viability was recorded. Up to 60 million cells (<6 aliquots) were frozen as a suspension in Cell Culture Freezing medium (Gibco #12648010), at up to 10 million cells per mL. Cells were stored at nominally −70° C. until shipped to on dry ice.

### Results

FIG. 5 shows spike protein antibody titer as determined day 21 and day 49 of the study. Various ionizable lipid containing ceDNA formulations (LNP1-6) were tested to determine if certain lipids were preferable to others in the formulations. As shown in FIG. 5, certain formulations were more immunogenic than the other LNP formulations tested (e.g., C7 ionizable lipid in combination with DOPE non-cationic lipid, cholesterol and DMG-PEG2000), indicating some lipids may be preferred over others in the ceDNA vaccine formulations.

### Example 7: Vaccination of a Subject by Administration of a DNA Priming Vaccine

In this example, mice were administered a ceDNA, mRNA, or plasmid priming vaccine encoding the COVID spike protein, followed by administration of an mRNA vaccine. ceDNA vectors were produced according to the methods described in Example 1 above. DNA plasmids and mRNA were produced as described herein and using routine methods known in the art.

FIG. 6 is a graph that depicts the percentage of IFNγ⁺ antigen-specific memory CD8⁺ T cells in mouse spleen cell suspensions 8 weeks after immunization with mRNA, ceDNA, or plasmid encoding the COVID spike protein. Mice were immunized with a priming dose of 3 μg of mRNA, ceDNA, or plasmid encoding the COVID spike protein (each formulated in LNPs). Eight weeks later, spleens were removed and processed into single-cell suspensions. The cells were stimulated with a library of spike protein epitopes to gauge the magnitude of the vaccine-induced immune response. The cells were stained for markers of memory CD8⁺ T cells and analyzed by flow cytometry. Within the CD8⁺ population, there is a statistically significantly larger fraction of vaccine-induced memory T effector cells (CD62L^{lo}CD44^{hi}CCR7^{lo}CD127^{hi}) in response to ceDNA than to mRNA or plasmid. These data suggest that ceDNA vaccination induces a larger memory T cell population than other modalities.

FIG. 7 is a graph that depicts the percentage of IFNγ⁺ antigen-specific memory CD8⁺ T cells in mice primed and boosted with ceDNA-ceDNA, mRNA-mRNA, or ceDNA-mRNA regimens. The spike protein-reactive CD8⁺ T cells were interrogated by flow cytometry (as described above) from mice primed and boosted at either 4, 6, or 8 week intervals. Measurements were taken 3 weeks after each respective boost. While all of the vaccine regimens and dose intervals elicited an immune response, the greatest population of vaccine-induced CD8⁺ T cells was seen in the ceDNA-mRNA regimen with 8 weeks between the first and second doses. This data suggests that the kinetics of antigen expression impacts memory T cell generation.

FIG. 8 is a graph that depicts the percentage of IFNγ⁺ antigen-specific memory CD8⁺ T cells after heterologous prime-boost regimens of 0.3 μg mRNA-3 μg mRNA, 1 μg mRNA-3 μg mRNA, 3 μg mRNA-3 μg mRNA, 3 μg ceDNA-3 μg mRNA, and 10 μg ceDNA-3 μg mRNA. Prime-boost regimens and measurements were done as described above. Modulating the priming dose of antigen exposure by titrating down the mRNA lessened the overall response, while increasing the priming dose of ceDNA had no impact, suggesting that the was at saturation. This data suggests that the ceDNA-mRNA heterologous prime-boost enhanced CD8⁺ T cell effect is related to expression kinetics from ceDNA as compared to mRNA, and not related to an effect that might arise from a low priming dose followed by a higher booster dose.

Various other assays can be performed to determine immune response, including determination of spike protein antibody titer.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A method of inducing an immune response against a first peptide and a second peptide in a subject, comprising:
administering a priming vaccine comprising a deoxyribonucleic acid (DNA) to the subject, wherein the DNA encodes a first peptide; and
administering a boosting vaccine comprising (i) a ribonucleic acid (RNA), wherein the RNA encodes the second peptide, or (ii) a second peptide to the subject,
thereby inducing the immune response against the first peptide and the second peptide in the subject.

2. The method of claim 1, wherein the priming vaccine comprises DNA encoding the first peptide and the boosting vaccine comprises RNA encoding the second peptide.

3. The method of claim 1, wherein the priming vaccine comprises DNA encoding the first peptide and the boosting vaccine comprises the second peptide.

4. The method of any one of claims 1-3, wherein the DNA comprises a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector.

5. The method of any one of claims 1-4, wherein the first and the second peptide are derived from a bacterial, a viral, a fungal or a parasitic infectious agent.

6. The method of any one of claims 1-5, wherein the first and the second peptide are derived from the same pathogenic organism.

7. The method of any one of claims 1-6, wherein the first and the second peptide are the same in the priming vaccine and the boosting vaccine.

8. The method of any one of claims 1-6, wherein at least one of the epitopes of the first and the second peptide are different in the priming and the boosting vaccine.

9. The method of any one of claims 1-8, wherein the DNA comprises a capsid-free closed ended DNA (ceDNA) vector comprising at least one nucleic acid sequence between flanking inverted terminal repeats (ITRs), wherein the at least one nucleic acid sequence encodes the peptide.

10. The method of any one of claims 1-9, wherein the first and/or the second peptide is a tumor associated antigen or is associated with an autoimmune condition.

11. The method any one of claims 1-10, wherein the first and/or the second peptide is selected from the group consisting of the peptides set forth in Tables 1-8.

12. The method of any one of claims 1-11, wherein the DNA comprises a promoter sequence linked to the at least one nucleic acid sequence.

13. The method of any one of claims 4-12, wherein the ceDNA vector comprises at least one poly A sequence.

14. The method of any one of claims 4-13, wherein the ceDNA vector comprises a 5′ UTR and/or an intron sequence.

15. The method of any one of claims 4-14, wherein the ceDNA vector comprises a 3′ UTR sequence.

16. The method of any one of claims 4-15, wherein the ceDNA vector comprises an enhancer sequence.

17. The method of any one of claims 9-16, wherein at least one of the flanking ITRs comprises a functional terminal resolution site and a Rep binding site.

18. The method of any one of claims 9-17, wherein one or both of the flanking ITRs are derived from a virus selected from the group consisting of a Parvovirus, a Dependovirus, and an adeno-associated virus (AAV).

19. The method of any one of claims 9-18, wherein the flanking ITRs are symmetric or asymmetric with respect to each other.

20. The method of any one of claims 9-19, wherein the flanking ITRs are symmetric or substantially symmetric.

21. The method of any one of claims 9-19, wherein the flanking ITRs are asymmetric.

22. The method of any one of claims 9-21, wherein one of the flanking ITRs is wild-type, or wherein both of the flanking ITRs are wild-type ITRs.

23. The method of any one of claims 9-22, wherein the flanking ITRs are from different viral serotypes.

24. The method of any one of claims 9-23, wherein the flanking ITRs are selected from the group consisting of the pairs of viral serotypes set forth in Table 8.

25. The method of any one of claims 9-24, wherein one or both of the flanking ITRs comprise a sequence selected from the group consisting of the sequences set forth in Table 9.

26. The method of any one of claims 9-25, wherein at least one of the flanking ITRs is altered from a wild-type AAV ITR sequence by a deletion, an addition, or a substitution that affects the overall three-dimensional conformation of the ITR.

27. The method of any one of claims 9-26, wherein one or both of the flanking ITRs are derived from an AAV serotype selected from the group consisting of AAV1, AAV2, AAV3, AAV4, AAV5, AAV6, AAV7, AAV8, AAV9, AAV10, AAV 11, and AAV12.

28. The method of any one of claims 9-27, wherein one or both of the flanking ITRs are synthetic.

29. The method of any one of claims 9-28, wherein one of the flanking ITRs is not a wild-type ITR, or wherein both of the ITRs are not wild-type ITRs.

30. The method of any one of claims 9-29, wherein one or both of the flanking ITRs are modified by a deletion, an insertion, and/or a substitution in at least one of the ITR regions selected from A, A′, B, B′, C, C′, D, and D′.

31. The method of claim 30, wherein the deletion, insertion, and/or substitution results in the deletion of all or part of a stem-loop structure formed by the A, A′, B, B′, C, or C′ regions.

32. The method of any one of claims 9-31, wherein one or both of the flanking ITRs are modified by a deletion, an insertion, and/or a substitution that results in the deletion of all or part of a stem-loop structure formed by the B and B′ regions.

33. The method of any one of claims 9-32, wherein one or both of the flanking ITRs are modified by a deletion, an insertion, and/or a substitution that results in the deletion of all or part of a stem-loop structure formed by the C and C′ regions.

34. The method of any one of claims 9-33, wherein one or both of the flanking ITRs are modified by a deletion, an insertion, and/or a substitution that results in the deletion of part of a stem-loop structure formed by the B and B′ regions and/or part of a stem-loop structure formed by the C and C′ regions.

35. The method of any one of claims 9-34, wherein one or both of the flanking ITRs comprise a single stem-loop structure in the region that, in a wild-type ITR, would comprise a first stem-loop structure formed by the B and B′ regions and a second stem-loop structure formed by the C and C′ regions.

36. The method of any one of claims 9-35, wherein one or both of the flanking ITRs comprise a single stem and two loops in the region that, in a wild-type ITR, would comprise a first stem-loop structure formed by the B and B′ regions and a second stem-loop structure formed by the C and C′ regions.

37. The method of any one of claims 9-36, wherein one or both of the flanking ITRs comprise a single stem and a single loop in the region that, in a wild-type ITR, would comprise a first stem-loop structure formed by the B and B′ regions and a second stem-loop structure formed by the C and C′ regions.

38. The method of any one of claims 9-37, wherein both flanking ITRs are altered in a manner that results in an overall three-dimensional symmetry when the ITRs are inverted relative to each other.

39. The method of any one of claims 1-38, wherein the DNA is delivered in a lipid nanoparticle (LNP).

40. The method of any one of claims 1-39, wherein the RNA is delivered in an LNP.

41. The method of any one of claims 1-40, wherein the RNA is a messenger RNA (mRNA).

42. The method of any one of claims 1-41, wherein the RNA comprises at least one nucleotide analogue.

43. The method of any one of claims 1-42, wherein the immune response is an antibody response.

44. The method of any one of claims 1-43, wherein the immune response is a T cell response.

45. The method of any one of claims 1-44, wherein the immune response is a memory (CD8⁺) T cell response.

46. The method of any one of claims 1-45, wherein the method comprises administering the boosting vaccine at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 14 weeks, at least about 16 weeks, at least about 1-2 weeks, at least about 2-3 weeks, at least about 3-4 weeks, at least about 4-5 weeks, at least about 5-6 weeks, at least about 6-7 weeks, at least about 7-8 weeks, at least about 8-9 weeks, at least about 9-10 weeks, at least about 10-11 weeks, at least about 11-12 weeks, at least about 12-13 weeks, at least 13-14 weeks, at least about 14-15 weeks, or at least about 15-16 weeks after administering the priming vaccine.

47. The method of any one of claims 1-46, wherein the method comprises administering the boosting vaccine about 8 weeks after administering the priming vaccine.

48. The method of any one of claims 1-47, wherein the interval between the administering of the priming vaccine and the administering of the boosting vaccine is at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, or at least about 112 days.

49. The method of any one of claims 1-48, wherein the interval between the administration of the priming vaccine and the administration of the boosting vaccine is about 64 days.

50. The method of any one of claims 1-49, wherein the method comprises administering two or more doses of the boosting vaccine to the subject.

51. The method of any one of claims 1-50, wherein the method comprises administering each dose of boosting vaccine at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 14 weeks, at least about 16 weeks, at least about 1-2 weeks, at least about 2-3 weeks, at least about 3-4 weeks, at least about 4-5 weeks, at least about 5-6 weeks, at least about 6-7 weeks, at least about 7-8 weeks, at least about 8-9 weeks, at least about 9-10 weeks, at least about 10-11 weeks, at least about 11-12 weeks, at least about 12-13 weeks, at least 13-14 weeks, at least about 14-15 weeks, or at least about 15-16 weeks after administering the previous vaccine.

52. The method of any one of claims 1-51, wherein the interval between the administering of the each dose of boosting vaccine and the administering of the previous vaccine is at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, at least about 58 days, at least about 59 days, at least about 60 days, at least about 61 days, at least about 62 days, at least about 63 days, at least about 64 days, at least about 65 days, at least about 66 days, at least about 67 days, at least about 68 days, at least about 69 days, at least about 70 days, at least about 71 days, at least about 72 days, at least about 73 days, at least about 74 days, at least about 75 days, at least about 76 days, at least about 77 days, at least about 78 days, at least about 79 days, at least about 80 days, at least about 81 days, at least about 82 days, at least about 83 days, at least about 84 days, at least about 85 days, at least about 86 days, at least about 87 days, at least about 88 days, at least about 89 days, at least about 90 days, at least about 91 days, at least about 92 days, at least about 93 days, at least about 94 days, at least about 95 days, at least about 96 days, at least about 97 days, at least about 98 days, at least about 99 days, at least about 100 days, at least about 101 days, at least about 102 days, at least about 103 days, at least about 104 days, at least about 105 days, at least about 106 days, at least about 107 days, at least about 108 days, at least about 109 days, at least about 110 days, at least about 111 days, or at least about 112 days.

53. The method of any one of claims 1-52, wherein the subject has a bacterial infection, a viral infection, a parasitic infection, or a fungal infection.

54. The method of any one of claims 1-53, wherein the subject has cancer.

55. The method of any one of claims 1-54, wherein the subject has an autoimmune disease or disorder.

56. The method of any one of claims 1-55, wherein one or more of the priming vaccine or the boosting vaccine comprises a pharmaceutically acceptable carrier.

57. The method of claim 56, wherein at least one of the priming vaccine and the boosting vaccine compositions further comprises an adjuvant.

58. The method of any one of claims 1-57, wherein at least one of the priming vaccine and the boosting vaccine is administered by a route selected from the group consisting of: intramuscular, intraperitoneal, buccal, inhalation, intranasal, intrathecal, intravenous, subcutaneous, intradermal, and intratumoral, or is administered to the interstitial space of a tissue.

59. A vaccine regimen comprising a priming vaccine comprising a deoxyribonucleic acid (DNA), wherein the DNA encodes a first peptide, followed by a boosting vaccine comprising (i) a ribonucleic acid (RNA) that encodes a second peptide, or (ii) a second peptide.

60. The vaccine regimen of claim 59, wherein the priming vaccine comprises an amount of DNA encoding an immunologically effective amount of the first peptide, and the boosting vaccine comprises an amount of RNA encoding an immunologically effective amount of the second peptide.

61. The vaccine regimen of claim 59, wherein the priming vaccine comprises an amount of DNA encoding an immunologically effective amount of the first peptide, and the boosting vaccine comprises an immunologically effective amount of the second peptide.

62. The vaccine regimen of any one of claims 59-61, wherein the DNA comprises a minicircle, a plasmid, a bacmid, a minigene, a ministring DNA (linear covalently closed DNA vector), a closed-ended linear duplex DNA (CELiD or ceDNA), a doggybone (dbDNA™) DNA, a dumbbell shaped DNA, a minimalistic immunological-defined gene expression (MIDGE)-vector, a viral vector or a nonviral vector.

63. The vaccine regimen of any one of claims 59-62, wherein the first peptide and the second peptide are derived from a bacterial infectious agent, a viral infectious agent, a fungal infectious agent, or a parasitic infectious agent.

64. The vaccine regimen of any one of claims 59-63, wherein the first peptide and the second peptide are derived from the same pathogenic organism.

65. The vaccine regimen of any one of claims 59-64, wherein the first peptide and the second peptide are the same in the priming vaccine and the boosting vaccine.

66. The vaccine regimen of any one of claims 59-64, wherein at least one of the epitopes of the first peptide and the second peptide are different in the priming and the boosting vaccine.

67. The vaccine regimen of any one of claims 59-66, wherein the DNA comprises a capsid-free closed ended DNA (ceDNA) vector comprising at least one nucleic acid sequence between flanking inverted terminal (ITRs), wherein the at least one nucleic acid sequence encodes the first peptide.

68. The vaccine regimen of any one of claims 59-62 or 65-67, wherein the first and/or the second peptide is a tumor associated antigen or is associated with an autoimmune condition.

69. The vaccine regimen of any one of claims 59-68, wherein the first and/or the second peptide is selected from the group consisting of the peptides set forth in Tables 1-8.

70. The vaccine regimen of any one of claims 59-69, wherein the DNA comprises a promoter sequence linked to the at least one nucleic acid sequence.

71. The vaccine regimen of any one of claims 62-70, wherein the ceDNA vector comprises at least one poly A sequence.

72. The vaccine regimen of any one of claims 62-71, wherein the ceDNA vector comprises a 5′ UTR and/or intron sequence.

73. The vaccine regimen of any one of claims 62-72, wherein the ceDNA vector comprises a 3′ UTR sequence.

74. The vaccine regimen of any one of claims 62-73, wherein the ceDNA vector comprises an enhancer sequence.

75. The vaccine regimen of any one of claims 67-74, wherein at least one of the flanking ITRs comprises a functional terminal resolution site and a Rep binding site.

76. The vaccine regimen of any one of claims 67-75, wherein one or both of the flanking ITRs are derived from a virus selected from a Parvovirus, a Dependovirus, and an adeno-associated virus (AAV).

77. The vaccine regimen of any one of claims 67-76, wherein the flanking ITRs are symmetric or asymmetric with respect to each other.

78. The vaccine regimen of any one of claims 67-77, wherein the flanking ITRs are symmetric or substantially symmetric.

79. The vaccine regimen of any one of claims 67-77, wherein the flanking ITRs are asymmetric.

80. The vaccine regimen of any one of claims 67-79, wherein one of the flanking ITRs is wild-type, or wherein both of the ITRs are wild-type ITRs.

81. The vaccine regimen of any one of claims 67-80, wherein the flanking ITRs are derived from different viral serotypes.

82. The vaccine regimen of any one of claims 67-81, wherein the flanking ITRs are selected from the group consisting of the viral serotypes set forth in Table 8.

83. The vaccine regimen of any one of claims 67-82, wherein one or both of the flanking ITRs comprises a sequence selected from the group consisting of the sequences set forth in Table 9.

84. The vaccine regimen of any one of claims 67-83, wherein at least one of the flanking ITRs is altered from a wild-type AAV ITR sequence by a deletion, an addition, or a substitution that affects the overall three-dimensional conformation of the ITR.

85. The vaccine regimen of any one of claims 67-84, wherein one or both of the flanking ITRs are derived from an AAV serotype selected from the group consisting of AAV1, AAV2, AAV3, AAV4, AAV5, AAV6, AAV7, AAV8, AAV9, AAV10, AAV11, and AAV12.

86. The vaccine regimen of any one of claims 67-85, wherein one or both of the flanking ITRs are synthetic.

87. The vaccine regimen of any one of claims 67-86, wherein one of the flanking ITRs is not a wild-type ITR, or wherein both of the flanking ITRs are not wild-type ITRs.

88. The vaccine regimen of any one of claims 67-87, wherein one or both of the flanking ITRs are modified by a deletion, an insertion, and/or a substitution in at least one of the ITR regions selected from A, A′, B, B′, C, C′, D, and D′.

89. The vaccine regimen of claim 88, wherein the deletion, insertion, and/or substitution results in the deletion of all or part of a stem-loop structure formed by the A, A′, B, B′, C, or C′ regions.

90. The vaccine regimen of any one of claims 67-89, wherein one or both of the flanking ITRs are modified by a deletion, an insertion, and/or a substitution that results in the deletion of all or part of a stem-loop structure formed by the B and B′ regions.

91. The vaccine regimen of any one of claims 67-90, wherein one or both of the flanking ITRs are modified by a deletion, an insertion, and/or a substitution that results in the deletion of all or part of a stem-loop structure formed by the C and C′ regions.

92. The vaccine regimen of any one of claims 67-91, wherein one or both of the flanking ITRs are modified by a deletion, an insertion, and/or a substitution that results in the deletion of part of a stem-loop structure formed by the B and B′ regions and/or part of a stem-loop structure formed by the C and C′ regions.

93. The vaccine regimen of any one of claims 67-92, wherein one or both of the flanking ITRs comprise a single stem-loop structure in the region that, in a wild-tye ITR, would comprise a first stem-loop structure formed by the B and B′ regions and a second stem-loop structure formed by the C and C′ regions.

94. The vaccine regimen of any one of claims 67-93, wherein one or both of the flanking ITRs comprise a single stem and two loops in the region that, in a wild-tye ITR, would comprise a first stem-loop structure formed by the B and B′ regions and a second stem-loop structure formed by the C and C′ regions.

95. The vaccine regimen of any one of claims 67-94, wherein one or both of the flanking ITRs comprise a single stem and a single loop in the region that, in a wild-tye ITR, would comprise a first stem-loop structure formed by the B and B′ regions and a second stem-loop structure formed by the C and C′ regions.

96. The vaccine regimen of any one of claims 67-95, wherein both flanking ITRs are altered in a manner that results in an overall three-dimensional symmetry when the ITRs are inverted relative to each other.

97. A method of treating a subject with a bacterial infection, a viral infection, a parasitic infection, or a fungal infection, comprising performing the method of any one of claims 1-58 or administering to the subject the vaccine regimen of any one of claims 59-96.

98. A method of treating a subject with a cancer, comprising performing the method of any one of claims 1-58 or administering to the subject the vaccine regimen of any one of claims 59-96.

99. A method of treating a subject with an autoimmune disease or disorder, comprising performing the method of any one of claims 1-58 or administering to the subject the vaccine regimen of any one of claims 59-96.

100. A method of preventing a bacterial infection, a viral infection, a parasitic infection, or a fungal infection in a subject, comprising performing the method of any one of claims 1-58 or administering to the subject the vaccine regimen of any one of claims 59-96.

101. A method of preventing cancer in a subject, comprising performing the method of any one of claims 1-58 or administering to the subject the vaccine regimen of any one of claims 59-96.

102. A method of preventing an autoimmune disease in a subject, comprising performing the method of any one of claims 1-58 or administering to the subject the vaccine regimen of any one of claims 59-96.

103. The method of any one of claims 97-102, wherein the method comprises administering two or more doses of the boosting vaccine to the subject.

104. The method of any one of claims 97-103, wherein the method comprises administering the boosting vaccine about 8 weeks after administering the priming vaccine.

105. The method of any one of claims 97-104, further comprising administering to the subject one or more additional therapeutic agents.

106. The vaccine regimen of any one of claims 59-96, wherein the priming vaccine and the boosting vaccine are each formulated in a pharmaceutical composition.

107. The vaccine regimen of claim 106, wherein one or both of the priming vaccine and the boosting vaccine further comprise one or more additional therapeutic agents.

108. The vaccine regimen of any one of claims 59-96 or 106-107, wherein one or both of the priming vaccine and the boosting vaccine further comprise a lipid.

109. The vaccine regimen of claim 108, wherein the lipid is a lipid nanoparticle (LNP).

110. The vaccine regimen of any one of claims 106-109, wherein one or both of the priming vaccine and the boosting vaccine are lyophilized.

111. A kit comprising the vaccine regimen of any one of claims 59-96 or 106-110, and instructions for use.
